(19) United States
(12) Patent Application Publication
(10) Pub. No.: US 2019/0183100 A1
(43) Pub. Date: Jun. 20, 2019

(54) ANIMAL MODELS FOR POLYCYSTIC KIDNEY DISEASE

(71) Applicant: Recombinetics, Inc., St. Paul, MN (US)

(72) Inventors: Daniel F. CARLSON, St. Paul, MN (US); Dennis WEBSTER, St. Paul, MN (US)

(21) Appl. No.: 16/226,391

(22) Filed: Dec. 19, 2018

Related U.S. Application Data
(60) Provisional application No. 62/607,724, filed on Dec. 19, 2017.

(51) Int. Cl. A01K 67/027

(52) U.S. Cl. CPC A01K 67/0276; A01K 2217/15; A01K 2217/075; A01K 2227/108; A01K 2267/0306; A01K 2217/203

(57) ABSTRACT

Disclosed herein are genomically edited livestock animals and in particular, swine for use as models of polycystic kidney disease (PKD) especially ARPKD and ADPKD. The animals disclosed herein are heterozygous, homozygous or compound heterozygous for mutations of the PKD1 and/or PKD2 genes and/or the PKHD1 gene. The mutations expressed in these models include mutations identified in humans responsible for PKD. In some embodiments, the expression is inducible. In further embodiments, the edit is introduced by an all in one expression cassette following the birth of the animal. Induction of the cassette allows for study of the model throughout the time course of the disease.

### CROSS-REFERENCE

This application claims the benefit of U.S. Provisional Application No. 62/607,724, filed Dec. 19, 2017, which is incorporated herein by reference in its entirety.

### STATEMENT AS TO FEDERALLY SPONSORED RESEARCH

This invention was made with the support of the United States government under Contract number 2R44DK104500-02 by the Small Business Innovation Research. The government may have rights to this disclosure.

### SEQUENCE LISTING

The instant application contains a Sequence Listing which has been submitted electronically in ASCII format and is hereby incorporated by reference in its entirety. Said ASCII copy, created on Dec. 19, 2018, is named 53545-730_201_SL.txt and is 27,610 bytes in size.

### FIELD OF THE INVENTION

The disclosure is directed to animal models of polycystic kidney disease especially swine.

### BACKGROUND

Polycystic kidney diseases (PKD) are a group of inherited disorders characterized by progressive cyst development in the kidney resulting in bilateral renal enlargement and often end stage renal disease (ESRD).

Autosomal dominant polycystic kidney disease (ADPKD) is the most common form of PKD (frequency 1:400-1000) and one of the most common monogenic diseases. Presently, there is no approved treatment in the US that can slow or reverse the growth of cysts and progression of the disease. Consequently, approximately half of patients with ADPKD advance to ESRD and require lifesaving kidney transplant or other renal replacement therapy by the age of 60.

Autosomal recessive polycystic kidney disease (ARPKD) affects approximately 1:20,000 live births. ARPKD causes kidney dysfunction and can lead to ESRD by the age of 40 and much earlier with severe loss of function mutations. ARPKD is most often lethal in pediatric cases and those that survive the pediatric period typically die of liver disease associated with PKHD1 mutations. Pathologies and symptoms of PKD include high blood pressure, headaches, abdominal pain, blood in the urine, and excessive urination. Other symptoms include pain in the back, and cyst formation (renal and other organs). Currently, there are no therapies proven to effectively prevent the progression of PKD.

### SUMMARY

Disclosed herein, in certain embodiments, is a genetically edited pig or dog as a model for studying autosomal recessive polycystic kidney disease (ARPKD), wherein the genome of the genetically edited pig or dog comprises at least one genetic edit to the PKHD1 gene and the genetically edited pig or dog expresses at least one phenotype associated with ARPKD.

In some embodiments, the genetic edit to the PKHD1 gene comprises biallelic mutations. In some embodiments, the genetic edit to the PKHD1 gene comprises a premature stop codon, a truncation mutation, or a missense mutation. In some embodiments, the pig or dog is homozygous for the genetic edit to the PKHD1 mutation. In some embodiments, the pig or dog is heterozygous for the genetic edit to the PKHD1 mutation. In some embodiments, the genetic edit to the PKHD1 gene comprises a T to M mutation at a position that corresponds to position 36 of the human amino acid sequence. In some embodiments, the genetic edit to the PKHD1 gene comprises an R to Q mutation at a position that corresponds to position 3240 of the human amino acid sequence. In some embodiments, the genetic edit to the PKHD1 gene results in a genotype of T36M/−, wherein 36 is a position that corresponds to position 36 of the human amino acid sequence and − is a knockout allele. In some embodiments, the genetic edit to the PKHD1 gene results in a genotype of T36M/T36M, wherein 36 is a position that corresponds to position 36 of the human amino acid sequence. In some embodiments, the genetic edit to the PKHD1 gene results in a genotype of −/−, wherein − is a knockout allele.

In some embodiments, the genetic edit to the PKHD1 gene results in a genotype of R3240Q/−, wherein 3240 is a position that corresponds to position 3240 of the human amino acid sequence and − is a knockout allele. In some embodiments, the genetic edit to the PKHD1 gene results in a genotype of R3240Q/R3240Q, wherein 3240 is a position that corresponds to position 3240 of the human amino acid sequence. In some embodiments, the genetic edit is made using an inducible system. In some embodiments, the inducible system is a tetracycline-dependent regulatory system or a Cre/loxP recombinase system. In some embodiments, the inducible system comprises a conditional allele cassette. In some embodiments, the conditional allele cassette comprises a wild-type exon preceding a mutant form of the exon facing the opposite direction and a CreER2 recombinase, wherein the conditional allele cassette is introduced into the PKHD1 gene.

In some embodiments, the inducible system is induced by administering tamoxifen or tetracycline. In some embodiments, the inducible system is induced when the pig or dog is in utero, about 1 day old, 2 days old, 3 days old, 1 week old, 2 weeks old, 3 weeks old, or 6 weeks old. In some embodiments, the at least one phenotype associated with ARPKD is selected from the group consisting of cysts in kidney, cysts in liver, cysts in seminal vesicles, high blood pressure, headaches, abdominal pain, blood in urine, excessive urination, back pain, changes in blood urea nitrogen (BUN) levels, creatinine, BUN/creatinine, and BUN/cAMP. In some embodiments, the at least one phenotype is present when the genetically edited pig or dog is at least about 6 weeks and older. In some embodiments, the at least one phenotype is present at least about 6 weeks and older after induction of the inducible system. In some embodiments, the pig is a mini-pig. In some embodiments, the mini-pig is selected from the group consisting of Ossabaw, Yucatan, Bama Xiang Zhu and Goettingen.

Disclosed herein, in certain embodiments, is a genetically edited pig blastocyst or dog blastocyst wherein the genome of the genetically edited pig blastocyst or dog blastocyst comprises at least one genetic edit to the PKHD1 gene.

Disclosed herein, in certain embodiments, is a method of making a genetically edited pig or dog for use as a model for studying autosomal recessive polycystic kidney disease (ARPKD), comprising: editing the PKHD1 gene to create a genetic edit to the PKHD1 gene wherein the genetically edited pig or dog expresses at least one phenotype associated with ARPKD.

In some embodiments, the editing of the PKHD1 gene comprises use of homology-independent targeted integration (HITI) or precise integration into target chromosome (PITCH) technology. In some embodiments, the editing of the PKHD1 gene comprises use of a nuclease. In some embodiments, the nuclease is a zinc-finger nuclease, a transcription activator-like effector nuclease (TALEN), a meganuclease, clustered regularly interspaced short palindromic repeats (CRISPR)/cas9, or CRISPR/Cpfl. In some embodiments, editing the PKHD1 gene comprises exposing a pig or dog embryo or a pig or dog primary somatic cell to a nuclease that edits the PKHD1 gene.

In some embodiments, the embryo disclosed herein is implanted into a surrogate mother to produce the genetically edited pig or dog for use as a model for studying ARPKD. In some embodiments, the primary somatic cell disclosed herein is cloned to produce an embryo which is implanted into a surrogate mother to produce the genetically edited pig or dog for use as a model for studying ARPKD. In some embodiments, the genetic edit to the PKHD1 gene comprises biallelic mutations. In some embodiments, the genetic edit to the PKHD1 gene comprises a premature stop codon, a truncation mutation, or a missense mutation. In some embodiments, the pig or dog is homozygous for the genetic edit to the PKHD1 mutation. In some embodiments, the pig or dog is heterozygous for the genetic edit to the PKHD1 mutation. In some embodiments, the genetic edit to the PKHD1 gene comprises a T to M mutation at a position that corresponds to position 36 of the human amino acid sequence. In some embodiments, the genetic edit to the PKHD1 gene comprises an R to Q mutation at a position that corresponds to position 3240 of the human amino acid sequence. In some embodiments, the genetic edit to the PKHD1 gene results in a genotype of T36M/−, wherein 36 is a position that corresponds to position 36 of the human amino acid sequence and − is a knockout allele. In some embodiments, the genetic edit to the PKHD1 gene results in a genotype of T36M/T36M, wherein 36 is a position that corresponds to position 36 of the human amino acid sequence.

In some embodiments, the genetic edit to the PKHD1 gene results in a genotype of −/−, wherein − is a knockout allele. In some embodiments, the genetic edit to the PKHD1 gene results in a genotype of R3240Q/−, wherein 3240 is a position that corresponds to position 3240 of the human amino acid sequence and − is a knockout allele. In some embodiments, the genetic edit to the PKHD1 gene results in a genotype of R3240Q/R3240Q, wherein 3240 is a position that corresponds to position 3240 of the human amino acid sequence. In some embodiments, the genetic edit is made using an inducible system. In some embodiments, the inducible system is tetracycline-dependent regulatory system or Cre/loxP recombinase system. In some embodiments, the inducible system comprises a conditional allele cassette. In some embodiments, the conditional allele cassette comprises a wild-type exon preceding a mutant form of the exon facing the opposite direction and a CreER2 recombinase, wherein the conditional allele cassette is introduced into the PKHD1 gene.

In some embodiments, the inducible system is induced by administering tamoxifen or tetracycline. In some embodiments, the inducible system is induced when the pig or dog is in utero, about 1 day old, 2 days old, 3 days old, 1 week old, 2 weeks old, 3 weeks old, or 6 weeks old. In some embodiments, the at least one phenotype associated with ARPKD is selected from the group consisting of cysts in kidney, cysts in liver, cysts in seminal vesicles, high blood pressure, headaches, abdominal pain, blood in urine, excessive urination, back pain, changes in blood urea nitrogen (BUN) levels, creatinine, BUN/creatinine, and BUN/cAMP. In some embodiments, the at least one phenotype is present when the genetically edited pig or dog is at least about 6 weeks and older. In some embodiments, the at least one phenotype is present at least about 6 weeks and older after induction of the inducible system.

In some embodiments, a method for evaluating an effect of a therapeutic treatment of ARPKD, comprises: providing the pig or dog disclosed herein, treating the pig or dog with a pharmaceutical composition, and evaluating the pig or dog for an effect of the pharmaceutical composition on a phenotype associated with ARPKD.

In some embodiments, a method for treating a human having at least one phenotype associated with ARPKD, comprises: providing the pig or dog disclosed herein, administering to the pig or dog a pharmaceutical composition; evaluating whether the pharmaceutical composition has an effect on the phenotype associated with ARPKD expressed by the pig or dog; and treating the human with the pharmaceutical composition if the pharmaceutical composition improves the phenotype associated with ARPKD expressed by the pig or dog.

Disclosed herein, in certain embodiments, is a genetically edited pig or dog as a model for studying autosomal dominant polycystic kidney disease (ADPKD), wherein the genome of the genetically edited pig or dog comprises a genetic edit to the PKD1 gene or PKD2 gene, or both PKD1 gene and PKD2 gene, wherein the genetic edit is induced by an inducible system and the genetically edited pig or dog expresses at least one phenotype associated with ADPKD after the inducible system is induced.

In some embodiments, the genetic edit is to the PKD1 gene. In some embodiments, the genetic edit comprises biallelic mutations. In some embodiments, the genetic edit comprises a premature stop codon, a truncation mutation, or a missense mutation to the PKD1 or PKD2 gene. In some embodiments, the pig or dog is homozygous for the genetic edit to the PKD1 or PKD2 mutation. In some embodiments, the pig or dog is heterozygous for the genetic edit to the PKD1 or PKD2 mutation. In some embodiments, the genetic edit to the PKD1 gene comprises an R to C mutation at a position that corresponds to position 3277 of the human amino acid sequence. In some embodiments, the genetic edit to the PKD1 gene comprises an R to W mutation at a position that corresponds to position 2220 of the human amino acid sequence. In some embodiments, the genetic edit to the PKD1 gene comprises an R to W mutation at a position that corresponds to position 2213 of the human amino acid sequence. In some embodiments, the genetic edit to the PKD1 gene results in a genotype of R3227C/R3227C, wherein 3277 is a position that corresponds to position 3277 of the human amino acid sequence. In some embodiments, the genetic edit to the PKD1 gene results in a genotype of R2220W/R2220W, wherein 2220 is a position that corresponds to position 2220 of the human amino acid sequence. In some embodiments, the genetic edit to the PKD1 gene results in a genotype of R2213W/R2213W, wherein 2213 is a position that corresponds to position 2213 of the human amino acid sequence. In some embodiments, the genetic edit to the PKD1 gene results in a genotype of +/−, wherein + is a wild-type allele and − is a knockout allele.

In some embodiments, the genetic edit to the PKD1 gene results in a genotype of R3227C/+, wherein 3277 is a position that corresponds to position 3277 of the human amino acid sequence and + is a wild-type allele. In some embodiments, the genetic edit to the PKD1 gene results in a genotype of R2220W/−, wherein 2220 is a position that corresponds to position 2220 of the human amino acid sequence, and − is a knockout allele. In some embodiments, the genetic edit to the PKD1 gene results in a genotype of R3227C/−, wherein 3277 is a position that corresponds to position 3277 of the human amino acid sequence and − is a knockout allele. In some embodiments, the inducible system is a tetracycline-dependent regulatory system or a Cre/loxP recombinase system. In some embodiments, the inducible system comprises a conditional allele cassette. In some embodiments, the conditional allele cassette comprises a wild-type exon preceding a mutant form of the exon facing the opposite direction and a CreER2 recombinase, wherein the conditional allele cassette is introduced into the PKHD1 gene. In some embodiments, the inducible system is induced by administering tamoxifen or tetracycline. In some embodiments, the inducible system is induced when the pig or dog is in utero, about 1 day old, 2 days old, 3 days old, 1 week old, 2 weeks old, 3 weeks old, or 6 weeks old.

In some embodiments, the at least one phenotype associated with ADPKD is selected from the group consisting of cysts in kidney, cysts in liver, cysts in seminal vesicles, high blood pressure, headaches, abdominal pain, blood in urine, excessive urination, back pain, changes in blood urea nitrogen (BUN) levels, creatinine, BUN/creatinine, and BUN/cAMP. In some embodiments, the at least one phenotype is present when the genetically edited pig or dog is at least 6 months old. In some embodiments, the at least one phenotype is present at least about 6 weeks or older after induction of the inducible system. In some embodiments, the pig is a mini-pig. In some embodiments, the mini-pig is selected from the group consisting of Ossabaw, Yucatan, Bama Xiang Zhu and Goettingen.

Disclosed herein, in certain embodiments, is a genetically edited pig blastocyst or dog blastocyst wherein the genome of the genetically edited pig blastocyst or dog blastocyst comprises a genetic edit to the PKD1 gene or PKD2 gene or both the PKD1 gene and PKD2 gene.

Disclosed herein, in certain embodiments, method of making a genetically edited pig or dog for use as a model for studying autosomal dominant polycystic kidney disease (ADPKD), comprising: editing the PKD1 gene or PKD2 gene or both the PKD1 and PKD2 gene to create a genetic edit, wherein the genetic edit is induced by an inducible system and the genetically edited pig or dog expresses at least one phenotype associated with ADPKD after the inducible system is induced.

In some embodiments, the genetic edit is to the PKD1 gene. In some embodiments, the editing of the PKD1 gene comprises use of homology-independent targeted integration (HITI) or precise integration into target chromosome (PITCh) technology. In some embodiments, the editing of the PKD1 gene comprises use of a nuclease. In some embodiments, the nuclease is a zinc-finger nuclease, a transcription activator-like effector nuclease (TALEN), a meganuclease, clustered regularly interspaced short palindromic repeats (CRISPR)/cas9, or CRISPR/Cpfl. In some embodiments, editing the PKD1 gene comprises exposing a pig or dog embryo or a pig or dog primary somatic cell to a nuclease that edits the PKD1 gene.

In some embodiments, the embryo disclosed herein is implanted into a surrogate mother to produce the genetically edited pig or dog for use as a model for studying ADPKD. In some embodiments, the primary somatic cell disclosed herein is cloned to produce an embryo which is implanted into a surrogate mother to produce the genetically edited pig or dog for use as a model for studying ADPKD. In some embodiments, the genetic edit comprises biallelic mutations. In some embodiments, the genetic edit comprises a premature stop codon, a truncation mutation, or a missense mutation to the PKD1 or PKD2 gene. In some embodiments, the pig or dog is homozygous for the genetic edit to the PKD1 or PKD2 mutation. In some embodiments, the pig or dog is heterozygous for the genetic edit to the PKD1 or PKD2 mutation. In some embodiments, the genetic edit to the PKD1 gene comprises an R to C mutation at a position that corresponds to position 3277 of the human amino acid sequence. In some embodiments, the genetic edit to the PKD1 gene comprises an R to W mutation at a position that corresponds to position 2220 of the human amino acid sequence. In some embodiments, the genetic edit to the PKD1 gene comprises an R to W mutation at a position that corresponds to position 2213 of the human amino acid sequence.

In some embodiments, the genetic edit to the PKD1 gene results in a genotype of R3227C/R3227C, wherein 3277 is a position that corresponds to position 3277 of the human amino acid sequence. In some embodiments, the genetic edit to the PKD1 gene results in a genotype of R2220W/R2220W, wherein 2220 is a position that corresponds to position 2220 of the human amino acid sequence. In some embodiments, the genetic edit to the PKD1 gene results in a genotype of R2213W/R2213W, wherein 2213 is a position that corresponds to position 2213 of the human amino acid sequence. In some embodiments, the genetic edit to the PKD1 gene results in a genotype of +/−, wherein + is a wild-type allele and − is a knockout allele. In some embodiments, the genetic edit to the PKD1 gene results in a genotype of R3227C/+, wherein 3277 is a position that corresponds to position 3277 of the human amino acid sequence and + is a wild-type allele. In some embodiments, the genetic edit to the PKD1 gene results in a genotype of R2220W/−, wherein 2220 is a position that corresponds to position 2220 of the human amino acid sequence, and − is a knockout allele. In some embodiments, the genetic edit to the PKD1 gene results in a genotype of R3227C/−, wherein 3277 is a position that corresponds to position 3277 of the human amino acid sequence and − is a knockout allele. In some embodiments, the inducible system is a tetracycline-dependent regulatory system or a Cre/loxP recombinase system.

In some embodiments, the inducible system comprises a conditional allele cassette. In some embodiments, the conditional allele cassette comprises a wild-type exon preceding a mutant form of the exon facing the opposite direction and a CreER2 recombinase, wherein the conditional allele cassette is introduced into the PKHD1 gene. In some embodiments, the inducible system is induced by administering tamoxifen or tetracycline. In some embodiments, the inducible system is induced when the pig or dog is in utero, about 1 day old, 2 days old, 3 days old, 1 week old, 2 weeks old, 3 weeks old, or 6 weeks old. In some embodiments, the at least one phenotype associated with ADPKD is selected from the group consisting of cysts in kidney, cysts in liver, cysts in seminal vesicles, high blood pressure, headaches, abdominal pain, blood in urine, excessive urination, back pain, changes in blood urea nitrogen (BUN) levels, creatinine, BUN/creatinine, and BUN/cAMP. In some embodiments, the at least one phenotype is present when the genetically edited pig or dog is at least 6 months old. In some embodiments, the at least one phenotype is present at least about 6 weeks and older after induction of the inducible system. In some embodiments, the pig is a mini-pig. In some embodiments, the mini-pig is selected from the group consisting of Ossabaw, Yucatan, Bama Xiang Zhu and Goettingen.

In some embodiments, a method for evaluating an effect of a therapeutic treatment of ADPKD, comprises: providing the pig or dog disclosed herein, treating the pig or dog with a pharmaceutical composition, and evaluating the pig or dog for an effect of the pharmaceutical composition on a phenotype associated with ADPKD.

In some embodiments, a method for treating a human having at least one phenotype associated with ARPKD, comprises: providing the pig or dog disclosed herein, administering to the pig or dog a pharmaceutical composition; evaluating whether the pharmaceutical composition has an effect on the phenotype associated with ADPKD expressed by the pig or dog; and treating the human with the pharmaceutical composition if the pharmaceutical composition improves the phenotype associated with ADPKD expressed by the pig or dog.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1: Shown is the sequence homology between human PKD1 and pig PKD1. There is 81.5% identity of the PC1 protein expressed by the PKD1 genes. The homology of the individual Exons 5, 15 and 29 as discussed herein is 72%, 81% and 88% respectively.

FIG. 2: Shown is the sequence homology between human PKDH1 and pig PKDH1. There is 76% identity of the FPC protein expressed by the PKDH1 gene. The homology of the individual Exons 3, 58, and 61 as discussed herein is 76%, 83% and 79% respectively.

FIG. 3A, FIG. 3B: Shown is gene editing demonstrated with TALENs. FIG. 3A shows a pair of TALENs executes a sequence specific double-strand DNA break (DSB) at the targeted locus. FIG. 3B shows that if no other DNA templates are added, the DSB is repaired by NHEJ, which can result in small insertions or deletions (indels; Example 1) that cause a frame-shift mutation. Alternatively, if a DNA sequence that has a high identity with the region surrounding the DSB is introduced, homology dependent repair (HDR) can occur. The introduced DNA sequence may vary by only a single (or a few) base pair, which results in a defined mutation or knockout allele (Example 2).

FIG. 4: Conditional expression of mutant PKD proteins in the pig. The PKD1^{FLEX-CreER2} is inserted directly into the pig PKD1 gene (ssPKD1) so wild type or mutant exons are incorporated into the mRNA in a conditional manner. The PKD1^{FLEX-CreER2} begins at the 5′ loxP site and ends at the Lox511 site. The cassette includes both wild type exon 15 (E15) and a mutant E15 in the opposite orientation, pictured here with the R2220W mutation. The cassette also includes EGFP and CreER² under control of the ubiquitous Ef1α promoter. Until tamoxifen is provided, CreER² is excluded from the nucleus and cannot stimulate recombination. In the wild type configuration above (before induction) splicing incorporates E14, E15 and E16 into the mRNA to produce a normal PKD1 protein. After induction with tamoxifen, CreER² performs 2 recombination reactions that first invert the cassette and then to remove the wild type E15 plus the EGFP-P2A-CreER². The use of incompatible loxP sites forces recombination only to the allele shown below that when spliced with the endogenous gene produces PKD1 protein with the R2220W mutation.

FIG. 5A, FIG. 5B, FIG. 5C, FIG. 5D, FIG. 5E: FIG. 5A, FIG. 5B, FIG. 5C show intra-locus multiplex gene-editing of pig PKD1. FIG. 5A) A schematic of the swine PKD1 gene is shown along with the locations of TALENs for introduction of null and hypomorphic alleles. FIG. 5B) Alignment of human and pig PKD1 proteins in exons 15 and 29 reveal cross-species conservation of R2213, R2220 and R3277. FIG. 5C) Alignment of human and pig PKD1 exon 5 shows cross-species sequence conservation and residues identified for stop/gain mutation. FIG. 5A, FIG. 5B, and FIG. 5C disclose SEQ ID NOS 110, 112, 111 and 113-117, respectively, in order of appearance. FIG. 5D and FIG. 5E show intra-locus multiplex gene-editing of pig PKDH1. FIG. 5D) A schematic of the swine PKDH1 gene is shown along with the locations of TALENs for introduction of null and hypomorphic alleles. FIG. 5E) Alignment of human and pig PKDH1 proteins in exons 3, 58 and 63 reveal cross-species conservation of T36, 13553 (V is similar to I) and R3240. It should be noted that throughout the application exon 58 is referred to as exon 65. This was based on a former contig now known to be an inaccurate annotation. Sequences, top bottom left to right: IEPEEGSLAGGTWITVIFDG (SEQ ID NO: 77); IEPEEGSLAGGTWITVFFDG (SEQ ID NO: 78); TSTDRAPSNPRGGRIGILWPVFTSEP (SEQ ID NO: 79); TSADRAPSSPRGGRVGILWPVFTSEP (SEQ ID NO: 80); VVLQGEEPIEIRSGVSIH (SEQ ID NO: 81); VVLQGEEPVEIRSGVAIH (SEQ ID NO: 82).

FIG. 6A, FIG. 6B, FIG. 6C, FIG. 6D: Intra-locus multiplex editing produces R2220W/− cells. FIG. 6A shows the sequence of templates for each allele. A novel restriction site, underlined, is engineered into each template to enable differential RFLP quantification of HDR on populations (as shown in FIG. 6B) and screening of colonies (as shown in FIG. 6C). Codons are highlighted in the N-R2220W template with intentional, silent mismatches that prevent re-cutting of the HDR allele. FIG. 6D shows Sanger sequencing confirms the intended R2220W/− genotype in clones 46 and 47.

FIG. 7A, FIG. 7B: RFLP assay on piglets born from a PKD1+/− sow bred with a PKD1+/R3277C boar. The exon 5 knockout allele was identified via HindIII restriction digest (as shown in FIG. 7A) and the R3277C allele was identified via AflIII restriction digest (as shown in FIG. 7B). RFLP bands were resolved by agarose gel electrophoresis (2%). Piglets 82-3, 82-SB1 and 82-SB4 were heterozygous for the knockout allele. Piglets 82-1, 82-SB2, 82-SB5 and 82-SAV were heterozygous for the R3277C allele.

FIG. 8A, FIG. 8B: Severe cystic phenotype in R3277C/null (RC/−) piglet. FIG. 8A shows the excised kidney from the 1-day old RC/null piglet in comparison to a 15 ml conical tube. FIG. 8B shows H&E staining of kidney from the RC/null piglet compared to tissue from a wild type piglet collected at the same age demonstrating the highly cystic architecture of the mutant kidney relative to wild type.

FIG. 9A, FIG. 9B, FIG. 9C, FIG. 9D: Overarching approach for production of ARPKD models via embryo injection. FIG. 9A shows a schematic of exon 3 in pigs with TALEN pairs indicated in red and CRISPR/Cas9 gRNA targets indicated in gray. A single stranded HDR template is utilized to introgress the T36M edit along with a novel restriction site for RFLP genotyping. FIG. 9B shows nucleases and HDR templates that were tested in fibroblasts and parthenogenetic embryos. FIG. 9C shows the best performing conditions are used to create founder animals by injection of in vitro fertilized zygotes and embryo transfer to surrogates. FIG. 9D shows a table of predicted genotypes: phenotypes from edited pigs. LOF=loss of function via indel formation, MS=random missense allele resulting from a read-through indel.

FIG. 10: High conservation of PKHD1 exon 3 between pigs and humans. The majority of exon 3 is displayed and tyrosine 36 is highlighted.

FIG. 11A, FIG. 11B: Evaluation of PDX1 TALENs in parthenogenetic pig embryos. FIG. 11A, shows the steps in production of in vitro matured pig embryos for microinjection. After injection, embryos are culture to the blastocyst stage prior to whole genome amplification and characterization of edits by RFLP assay (for HDR) and sequencing. FIG. 11B shows the results of a dosage escalation study using TALENs targeted to PDX1.

FIG. 12A, FIG. 12B: T36M introgression in cells. FIG. 12A shows a protein alignment of pig and human proteins in exon 3. The region is highly conserved and threonine acid at position 36 in humans corresponds to residue 36 in pigs. Threonine acid 36 is identified in pig genomic DNA sequence, and one CRISPR (maroon arrow) are designed to cleave near T36. FIG. 12A also discloses SEQ ID NO: 118. FIG. 12B shows large white male fibroblasts were transfected with 1 μg of CRISPR mRNA along with 0.2 nMol of the T36M HDR template, or TALENs targeted to exon 61 for introgression of the human I3553T mutation. It should be noted that position 3553 in pigs is a Valine residue and not pathogenic; henceforth this mutation will be referred to as either I3553T or V3553T. The cells were recovered for three days at 30 and 37 degrees C. prior to quantification of activity by the Surveyor assay and RFLP analysis with the Nsp1 restriction enzyme. The arrowheads depict the expected size of cleavage products for the Surveyor assay or RFLP assay.

FIG. 13A, FIG. 13B: FIG. 13A and FIG. 13B show RFLP analysis and Sanger sequencing of single parthenogenetic blastocysts. FIG. 13A shows mutant bands (224 and 236 bp) after digestion of PCR products when Cas9/gRNA was injected together with HDR template at a concentration of 100/50/50 (ng/μl). FIG. 13B shows that the genotype of all embryos confirmed results of the restriction assay. Three examples are shown for the validation of the genotype through sequencing.

FIG. 14A, FIG. 14B, FIG. 14C: Shown are photographs of embryos at 29 days of gestation following injections of CRISPRs/HDR Oligo into recipient sows 1277 (FIG. 14A), 1278 (FIG. 14B) and 1279 (FIG. 14C).

FIG. 15A, FIG. 15B, FIG. 15C: Shown are gels showing the RFLP analysis of the embryos shown in FIG. 14A, FIG. 14B and FIG. 14C. U=undigested; D=digested by restriction enzyme.

FIG. 16A, FIG. 16B: FIG. 16A is an alignment of exon in the PKHD1 gene of humans and swine showing the homology the R3240 and R3229 residues in human and swine. FIG. 16B shows the strategy for introducing the R3229Q mutation in swine as well as altering the sequence to include a silent mutation for glycine to comprise an EcoR1 site used for RFLP analysis. FIG. 16B also discloses SEQ ID NO: 119.

FIG. 17: Shown is a gel showing RFLP analysis of embryos injected with gene editing reagents. Asterisks indicate introgression of the mutation, red asterisks indicate homozygous introgression of the mutation. Screening primers ssPKHD1 E65 F2 and R2 generate a 443-base pair amplicon with RFLP bands of 228 and 215 when the HDR event has occurred.

FIG. 18: Sequence confirmation of two blastocysts from the C3 condition. Blastocyst 3.19 is homozygous for R3229Q while 3.12 has one HDR allele and one indel allele.

FIG. 19: Kidneys and livers from newborn WT or ARPKD models. These animals are littermates, the ARPKD models having null alleles of PKHD1.

FIG. 20A, FIG. 20B: Shown are magnetic resonance images of four ARPKD models presented over a time course of 4 months. FIG. 20A shows magnetic resonance images of ARPKD models that were imaged at 3-6 months of age. FIG. 20B shows ex-vivo MR imaging of kidneys from the same animals.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

ADPKD is genetically heterogeneous with two loci identified, PKD1 (16p13.3) and PKD2 (4q21). PKD1 is a large gene (46 exons) with a coding region of ˜13 kb, while PKD2 (15 exons) has a ˜3 kb coding region. PKD1 and PKD2 encode polycystin-1 (PC1) and -2 (PC2), respectively. PC2 is a transient receptor potential (TRP), cation channel and PC1 is a receptor protein that complexes with PC2 to form a functional channel. Specific cleavage of PC1 is an important step to form functional PC1 with mature, membrane bound, and immature, ER products, generated. Cilia are thought central to the pathogenesis in PKD; it is a ciliopathy. Urinary microvesicles, which contain high levels of the PCs, may also be important for disease development.

ARPKD is a recessive form of PKD. ARPKD is associated with mutations in the PKHD1 gene (6p12.2 in humans). In humans, PKHD1 is one of the largest genes in the genome, occupying approximately 450 kb of DNA and contains at least 86 exons. The largest known transcript encodes fibrocystin/polyductin (FPC), which is a large receptor-like integral membrane protein of 4074 amino acids. The structure of the FPC consist of a single transmembrane, a large N-terminal extracellular region, and a short intracellular cytoplasmic domain. The FPC protein is found on the primary cilia of epithelia cells of cortical and medullary collecting ducts and cholangiocytes of bile ducts, and show similarity to polycystins and several other ciliopathy proteins. FPC is also found to be expressed on the basal body and plasma membrane. It is presumed that the large extracellular domain of FPC binds to a ligand(s) that is yet unknown and that is also involved in cell-cell and cell-matrix interactions. There have been a large number of various single gene mutations found throughout PKHD1 and are unique to individual families. Most of the patients are compound heterozygotes for PKHD1 mutations. Patients with two nonsense mutations appear to have an earlier onset of the disease.

A number of rodent models exist for PKD that have been critical for the characterization of disease pathology and understanding mechanisms of disease related to different genotypes. However, there is mounting evidence that response to treatment in rodents is a poor indicator of success in humans due to key differences in renal physiology. To fill this unmet need, the instant disclosure is focused on the development of swine models for PKD. In contrast to rodents, the anatomy, biochemistry, physiology, size, and genetics of pigs resemble those of humans, particularly relating to urological systems.

Polycystic kidney diseases (PKD) are a group of inherited disorders resulting in cyst development in the kidneys, but can also include a range of extra-renal manifestations. The most common form is autosomal dominant polycystic kidney disease (ADPKD) affecting approximately 1 in 500 live births and is one of the most common monogenic diseases. A second form, autosomal recessive polycystic kidney disease (ARPKD) affects 1 in 20,000 live births. In both ADPKD and ARPKD, the disease is characterized by progressive cyst formation resulting in bilateral renal enlargement and often end stage renal disease (ESRD) while ARPKD often progresses to ESRD in survivors. An estimated ˜30,000 US patients have ESRD due to ADPKD, with 4-10% of patients requiring renal replacement therapy. The disease develops over the lifetime of the patient—microcysts can be detected in utero and development of new cysts and cystic expansion occurs throughout the patient's life. Patients typically develop hypertension at a relatively young age (20-30), have an early urinary concentrating defect, and often suffer from pain. ESRD occurs from the 4^{th} decade. Autosomal recessive (ARPKD) is a devastating disease that strikes early, in fact up to 30% of ARPKD patients die as neonates. As many as 45% of those surviving the first month of life are prone to end stage renal disease, whereas those that live past 15 years old often succumb to liver disease.

Current models of PKD in rats and mice are less than optimal, particularly for translation of treatment to the clinic; hence, there is a significant unmet need for a large animal model that better replicates human physiology to facilitate new treatment approaches. Disclosed herein are viable pig models that manifest with the targeted morbidity while providing a lifespan conducive to research gains. However, the challenge is to compress into just six months the typical human disease course—from cyst initiation to severe cystic disease that normally develops over decades. To solve this, innovative gene-editing platforms were used to prototype four distinct model genotypes in pigs (PKD1^{R2220W/-}; PDK1^{R3277C/-}; PDK1^{+/R3277C} and PKD1^{+/-}), predicted to lower the level of functional polycystin-1 protein (PC1) to 10-50% of normal. Severe disease was found in compound heterozygous founders, the first evidence that severe PKD can develop in pigs, whereas heterozygotes had very mild disease. A breeding herd of PKD1^{+/R3277C} and PKD1^{+/-} was established from which two models, PDK1^{R3277C/-} (RC-null) and PKD1^{R3277C/R3277C} (RC-RC) are bred. One embodiment comprises the first inducible model of PKD in pig models to enable therapeutic testing at very early stages of disease development. These novel PKD models are produced and characterized with state-of-the art clinical procedures to establish benchmark data for researchers and clinicians.

A similar approach was adopted for ARPKD where the genotype:phenotype relationship is somewhat stochastic, and a survey of multiple knockout or missense mutation phenotypes is required to establish models with predictable disease onset. This can be achieved by making combinations of knockout, missense, yielding mild to severe hypomorphic mutations, in single animals. This can be achieved by cytoplasmic injection of pig zygotes with gene-editing reagents; for example, targeting exon 3 of PKHD1 results in several combinations of ARPKD alleles from the most prevalent T36M allele to frame-shift and read-through indel alleles. This ad hoc approach could be used routinely to produce pigs with a range of ARPKD severity from a single litter- or produce the founder animals for breeding of the study population. Artisans will appreciate that any other commonly mutated residues in ARPKD can be targeted in the same manner as above with a similar expected outcome. The severity and onset of disease is predicted to correlate with the pathogenicity of the mutation where null mutations are most severe and missense mutations can have a range of severity depending on the residues altered; for example, the alleles demonstrated in the present application, T36M, V3553T, R3229Q.

### Definitions

The terminology used herein is for the purpose of describing particular cases only and is not intended to be limiting. Unless defined otherwise, all technical and scientific terms used herein have the same meanings as commonly understood by one of ordinary skill in the art to which this disclosure belongs.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” refers to an amount that is near the stated amount by about 10%, 5%, or 1%, including increments therein. For example, “about” or “approximately” can mean a range including the particular value and ranging from 10% below that particular value and spanning to 10% above that particular value.

“Additive Genetic Effects” as used herein means average individual gene effects that can be transmitted from parent to progeny.

“Allele,” as used herein, refers to an alternate form of a gene. It also can be thought of as variations of DNA sequence. For instance, if an animal has the genotype for a specific gene of Bb, then both B and b are alleles.

“DNA Marker,” as used herein, refers to a specific DNA variation that can be tested for association with a physical characteristic.

“Genotype,” as used herein, refers to the genetic makeup of an animal.

“Genotyping (DNA marker testing),” as used herein, refers to the process by which an animal is tested to determine the particular alleles it is carrying for a specific genetic test.

“Homozygous,” as used herein, refers to having two copies of the same allele for a single gene such as BB.

“Heterozygous,” as used herein, refers to having a single mutant allele in a diploid genome such as Bb.”

“Compound heterozygous,” as used herein, means having two different mutant alleles for a gene in a diploid genome.

“Locus” (plural “loci”),” as used herein, refers to the specific locations of a maker or a gene.

“Chromosomal crossover” (“crossing over” or “crossover”),” as used herein, is the exchange of genetic material between homologous chromosomes inherited by an individual from its mother and father. Each individual has a diploid set (two homologous chromosomes, e.g., 2n) one each inherited from its mother and father. During meiosis I the chromosomes duplicate (4n) and crossover between homologous regions of chromosomes received from the mother and father may occur resulting in new sets of genetic information within each chromosome. Meiosis I is followed by two phases of cell division resulting in four haploid (1n) gametes each carrying a unique set of genetic information. Because genetic recombination results in new gene sequences or combinations of genes, diversity is increased. Crossover usually occurs when homologous regions on homologous chromosomes break and then reconnect to the other chromosome.

“Marker Panel,” as used herein, a combination of two or more DNA markers that are associated with a particular trait.

“Nucleotide,” as used herein, refers to a structural component of DNA that includes one of the four base chemicals: adenine (A), thymine (T), guanine (G), and cytosine (C).

“Phenotype,” as used herein, refers to the outward appearance of an animal that can be measured. Phenotypes are influenced by the genetic makeup of an animal and the environment.

“Single Nucleotide Polymorphism (SNP),” as used herein, is a single nucleotide change in a DNA sequence.

“Haploid genotype” or “haplotype,” as used herein, refer to a combination of alleles, loci or DNA polymorphisms that are linked so as to co-segregate in a significant proportion of gametes during meiosis. The alleles of a haplotype may be in linkage disequilibrium (LD).

“Hypomorph,” as used herein refers a mutation that causes a partial loss of gene function. A hypomorph is a reduction in gene function through reduced (protein, RNA) expression or reduced functional performance, but not a complete loss. The phenotype of a hypomorph is more severe in trans to a deletion allele than when homozygous. m/DF >m/m Hypomorphs are usually recessive, but occasional alleles are dominant due to haploinsufficiency.

“Haploinsufficiency,” as used herein, is a mechanism of action to explain a phenotype when a diploid organism has lost one copy of a gene and is left with a single functional copy of that gene. Haploinsufficiency is often caused by a loss-of-function mutation, in which having only one copy of the wild-type allele is not sufficient to produce the wild-type phenotype.

“Linkage disequilibrium (LD),” as used herein, is the non-random association of alleles at different loci i.e., the presence of statistical associations between alleles at different loci that are different from what would be expected if alleles were independently, randomly sampled based on their individual allele frequencies. If there is no linkage disequilibrium between alleles at different loci they are said to be in linkage equilibrium.

The terms “restriction fragment length polymorphism” or “RFLP,” as used herein, refer to any one of different DNA fragment lengths produced by restriction digestion of genomic DNA or cDNA with one or more endonuclease enzymes, wherein the fragment length varies between individuals in a population.

“Introgression” also known as “introgressive hybridization,” as used herein, is the movement of a gene or allele (gene flow) from one species into the gene pool of another by the repeated backcrossing of an interspecific hybrid with one of its parent species. Purposeful introgression is a long-term process; it may take many hybrid generations before the backcrossing occurs.

“Nonmeiotic introgression,” as used herein, is genetic introgression via introduction of a gene or allele in a diploid (non-gametic) cell. Non-meiotic introgression does not rely on sexual reproduction and does not require backcrossing and, significantly, is carried out in a single generation. In non-meiotic introgression an allele is introduced into a haplotype via homologous recombination. The allele may be introduced at the site of an existing allele to be edited from the genome or the allele can be introduced at any other desirable site.

As used herein, the term “genetic modification” or “genetic editing” refers to the direct manipulation of an organism's genome using biotechnology.

“Transcription activator-like effector nucleases (TALENs),” as used herein, is a technology for gene editing are artificial restriction enzymes generated by fusing a TAL effector DNA-binding domain to a DNA cleavage domain.

“Zinc finger nucleases (ZFNs),” as used herein, are another technology useful for gene editing and are a class of engineered DNA-binding proteins that facilitate targeted editing of the genome by creating double-strand breaks in DNA at user-specified locations.

“Meganuclease,” as used herein, are another technology useful for gene editing and are endodeoxyribonucleases characterized by a large recognition site (double-stranded DNA sequences of 12 to 40 base pairs); as a result, this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

“CRISPR/Cas” technology, as used herein, refers to “CRISPRs” (clustered regularly interspaced short palindromic repeats), segments of prokaryotic DNA containing short repetitions of base sequences. Each repetition is followed by short segments of “spacer DNA” from previous exposures to a bacterial virus or plasmid. “Cas9” (CRISPR associated protein 9) is an RNA-guided DNA endonuclease enzyme associated with the CRISPR. By delivering the Cas9 protein and appropriate guide RNAs into a cell, the organism's genome can be cut at any desired location.

“Indel,” as used herein, is shorthand for “insertion” or “deletion” referring to an edit of the DNA in an organism.

“Genetic marker,” as used herein, refers to a gene/allele or known DNA sequence with a known location on a chromosome. The markers may be any genetic marker e.g., one or more alleles, haplotypes, haplogroups, loci, quantitative trait loci, or DNA polymorphisms [restriction fragment length polymorphisms (RFLPs), amplified fragment length polymorphisms (AFLPs), single nuclear polymorphisms (SNPs), indels, short tandem repeats (STRs), microsatellites and minisatellites]. Conveniently, the markers are SNPs or STRs such as microsatellites, and more preferably SNPs. Preferably, the markers within each chromosome segment are in linkage disequilibrium.

As used herein, the term “host animal” means an animal which has a native genetic complement of a recognized species or breed of animal.

As used herein, “native haplotype” or “native genome” means the natural DNA of a particular species or breed of animal that is chosen to be the recipient of a gene or allele that is not present in the host animal.

As used herein, the term “target locus” means a specific location of a known allele on a chromosome.

As used herein, the term “quantitative trait” refers to a trait that fits into discrete categories. Quantitative traits occur as a continuous range of variation such as that amount of milk a particular breed can give or the length of a tail. Generally, a larger group of genes controls quantitative traits.

As used herein, the term “qualitative trait” is used to refer to a trait that falls into different categories. These categories do not have any certain order. As a general rule, qualitative traits are monogenic, meaning the trait is influenced by a single gene. Examples of qualitative traits include blood type and flower color, for example.

As used herein, the term “quantitative trait locus (QTL)” is a section of DNA (the locus) that correlates with variation in a phenotype (the quantitative trait).

As used herein, the term “cloning” means production of genetically identical organisms asexually.

“Somatic cell nuclear transfer” (“SCNT”), as used herein, is one strategy for cloning a viable embryo from a body cell and an egg cell. The technique consists of taking an enucleated oocyte (egg cell) and implanting a donor nucleus from a somatic (body) cell.

“Orthologous,” as used herein, refers to a gene with similar function to a gene in an evolutionarily related species. The identification of orthologues is useful for gene function prediction. In the case of livestock, orthologous genes are found throughout the animal kingdom and those found in other mammals may be particularly useful for transgenic replacement. This is particularly true for animals of the same species, breed or lineages wherein species are defined two animals so closely related as to being able to produce fertile offspring via sexual reproduction; breed is defined as a specific group of domestic animals having homogenous phenotype, homogenous behavior and other characteristics that define the animal from others of the same species; and wherein lineage is defined as continuous line of descent; a series of organisms, populations, cells, or genes connected by ancestor/descendent relationships. For example, domesticated cattle are of two distinct lineages both arising from ancient aurochs. One lineage descends from the domestication of aurochs in the Middle East while the second distinct lineage descends from the domestication of the aurochs on the Indian subcontinent.

“Genotyping” or “genetic testing,” as used herein, generally refers to detecting one or more markers of interest e.g., SNPs in a sample from an individual being tested, and analyzing the results obtained to determine the haplotype of the subject. As is apparent from the disclosure herein, it is one exemplary embodiment to detect the one or more markers of interest using a high-throughput system comprising a solid support consisting essentially of or having nucleic acids of different sequence bound directly or indirectly thereto, wherein each nucleic acid of different sequence comprises a polymorphic genetic marker derived from an ancestor or founder that is representative of the current population and, more preferably wherein said high-throughput system comprises sufficient markers to be representative of the genome of the current population. Preferred samples for genotyping comprise nucleic acid, e.g., RNA or genomic DNA and preferably genomic DNA. A breed of livestock animal can be readily established by evaluating its genetic markers.

Disclosed herein, in certain embodiments, is a genetically edited pig or dog as a model for studying autosomal recessive polycystic kidney disease (ARPKD), wherein the genome of the genetically edited pig or dog comprises at least one genetic edit to the PKHD1 gene and the genetically edited pig or dog expresses at least one phenotype associated with ARPKD.

Disclosed herein, in certain embodiments, is a genetically edited pig or dog as a model for studying autosomal dominant polycystic kidney disease (ADPKD), wherein the genome of the genetically edited pig or dog comprises a genetic edit to the PKD1 gene or PKD2 gene, or both PKD1 gene and PKD2 gene, wherein the genetic edit is induced by an inducible system and the genetically edited pig or dog expresses at least one phenotype associated with ADPKD after the inducible system is induced.

### Polycystic Kidney Disease (PKD)

A number of rodent models exist for PKD that have been critical for the characterization of disease pathology and understanding mechanisms of disease related to different genotypes. However, there is mounting evidence that response to treatment in rodents is a poor indicator of success in humans due to key differences in renal physiology. Symptoms and/or phenotypes of PKD in humans include: structural abnormalities of renal tubules, cysts in kidney, liver, seminal vesicles or pancreas, aneurysms, especially of the aortic root and circle of Willis hypertension, cyst infections, urinary bleeding and declining renal function

To fill this unmet need, the present disclosure provides swine models for PKD. In contrast to rodents, the anatomy, biochemistry, physiology, size, and genetics of pigs resemble those of humans, particularly relating to urological systems. However, the challenge was to compress into just six months the typical human disease course—from cyst initiation to severe cystic disease that normally develops over decades. To solve this, an innovative gene-editing platform was used to prototype four distinct model genotypes in pigs (PKD1^{R2220W/-}; PDK1^{R3277C/-}; PDK1^{+/R3277C} and PKD1^{+/-}), predicted to lower the level of functional polycystin-1 protein (PC1) to 10-50% of normal. These methods identified severe disease in compound heterozygous founders, the first evidence that severe PKD can develop in pigs, whereas heterozygotes had very mild disease. A breeding herd of PKD1^{+/R3277C} and PKD1^{+/-} was established; from which two models, PKD1^{R3277C/-} (RC-null) and PKD1^{R3277C/R3277C} (RC-RC) were bred. This enables the generation of the first inducible model of PKD in pigs to enable therapeutic testing at very early stages of disease development. The following examples teach how to produce and characterize these novel PKD models with state-of-the art clinical procedures to establish benchmark data for use in therapeutic and pharmaceutical testing as well as treatment modalities and palliative care.

ADPKD is genetically heterogeneous with two loci identified in humans, PKD1 (16p13.3) and PKD2 (4q21). PKD1 is a large gene (46 exons) with a coding region of ˜13 kb, while PKD2 (15 exons) has a ˜3 kb coding region. PKD1 and PKD2 encode polycystin-1 (PC1) and -2 (PC2), respectively. PC2 is a transient receptor potential (TRP), cation channel and PC1 a receptor protein that complexes with PC2 to form a functional channel. Specific cleavage of PC1 is an important step to form functional PC1 with mature, membrane bound, and immature, ER products, generated. Cilia are thought central to the pathogenesis in PKD; it is a ciliopathy. Urinary microvesicles, which contain high levels of the PCs, may also be important for disease development.

### Genetics of ADPKD

In cohorts identified via the renal clinic, PKD1 accounts for ˜80% of pedigrees, PKD2 ˜13%, with no mutation detected (NMD) in ˜7%. Any single, fully inactivating mutation to PKD1 or PKD2 causes ADPKD. PKD1 is a significantly more severe disease, with an average age at ESRD of 58.1y compared to 79.7y for PKD2. Recently, up to 50% of non-truncating changes have been recognized as incompletely penetrant alleles (hypomorphic); resulting in ESRD at 55y in patients with truncating PKD1 mutations and 67y for those with in-frame changes. To understand the role hypomorphic alleles play, several families with combinations of inactivating and hypomorphic PKD1 alleles have been characterized.

The best-characterized allele is PKD1;p.R3277C, where homozygotes develop adult onset ADPKD, while heterozygotes have just a few cysts. In two other families with in utero onset ADPKD, a truncating PKD1 mutation, or a second hypomorphic allele, PKD1; p.R2220W, in trans with pR3277C, was the cause of the severe disease. A knock-in mouse mimicking this allele (Pkd1^{R3277C (RC)}) is described in detail below.

The PKD1;p.R2220W hypomorphic allele is employed here to generate a knock-in pig model of ADPKD since the human studies indicate this is a strongly penetrant hypomorphic allele. The mechanism of how these hypomorphic alleles cause ADPKD has been revealed from in vitro and in vivo studies showing that the level of the mature, plasma membrane/cilia localized PC1, is critical. Both alleles reduce the efficiency of the normal cleavage of PC1 at the GPS domain, which is a vital step to activate PC. In addition, PKD1;p.R3277C has a folding defect that can be rescued by growing cells at a lower temperature. These studies of hypomorphic alleles have led to a model in ADPKD where cysts develop below a particular threshold of PC1 expression and that the severity of cystic disease is associated with the level of functional PC1. This dosage hypothesis is the basis for generating the PKD1 pig models described here.

### Development and Utilization of ADPKD Model Systems

Homozygous null Pkd1 or Pkd2 mice are embryonic lethal (˜E14.5), while null heterozygotes develop very few cysts even in old age. Hence, these models are of limited value for mimicking the human disease and are not suitable for preclinical testing. Conditional mouse models, where disease onset is induced at specific times and/or in specific organs, show an important switch around the time when murine renal development is complete (˜P13). If PC1 is inactivated before 13 days of age (P13), very rapidly progressive cystic disease develops, while activation after P13 results in a very slowly progressive disease.

There is also evidence that renal injury promotes cyst development/growth, indicating that proliferation likely influences the rate of cyst development. While these murine models have been useful to understand disease development and have been employed for preclinical testing, they do not fully reflect the disease pathogenesis in humans since all PC1 is removed at one time. Furthermore, clinically relevant models are difficult to generate in species where ES cells are not available because of the multiple changes required at the disease locus and the need for suitable kidney specific and/or inducible Cre transgenic animals. An alternative strategy, more suitable for generating large animal models by gene editing, is to generate a hypomorphic model. Earlier studies demonstrate that lowering PC1 levels to 15-20% of normal, leads to very rapid cystic expansion for one month with a subsequent reduction in kidney size as fibrosis develops. Mimicking the Pkd1;p.R3277C hypomorphic variant, that lowers the level of functional PC1 to ˜40% of normal, has been shown to develop a model better matching the progressive onset of human ADPKD. In this case, homozygous mice develop slowly progressive disease (cystic expansion up to 9 months and viability to >18 months), while the compound heterozygote with a null allele develops rapidly progressive disease with massive cystic expansion by one month. The mouse Pkd1;p.R3277C model, where disease develops from a consistent reduction of PC1 level has enabled the nephron region that becomes cystic and the effect on cilia structure to be analyzed. In the forgoing experiments, a variety of PKD1 hypomorphic mutations were mimicked in combination with null alleles as homozygotes to narrow in on the combination required to produce early onset disease without premature morbidity.

Beyond rodents, a number of large animals spontaneously develop ADPKD due to PKD1 mutation have been identified. For Persian cats, ˜40% worldwide develop PKD due to a PKD1 nonsense mutation in exon 29. These animals have mild PKD with renal insufficiency only occurring in old cats (>7 years) with only few, small cysts seen in younger cats. Likewise, PKD is common in Bull Terriers due to a non-conservative missense mutation in PKD1 exon 29 and results in slowly progressive disease. The mild nature of the disease in these common pets does not make them ideal for preclinical studies. Little work has yet been described in the pig—although the PKD1 gene has been fully characterized and PKD2 has been expressed transgenically in the pig, with no phenotype detected in younger animals. There is one spontaneous example of PKD in large white swine that originates from a common boar and is inherited in an autosomal dominant manner. Although the molecular etiology is unknown, it is likely similar to the result of heterozygous inactivation of PKD1 or PKD2. Cysts were observed in young pigs, <6 months, but the disease was still in an early state of progression consistent with the hypothesis that more than one mutant allele in combination is required to develop a rapidly progressive model.

### Therapeutic Testing for ADPKD

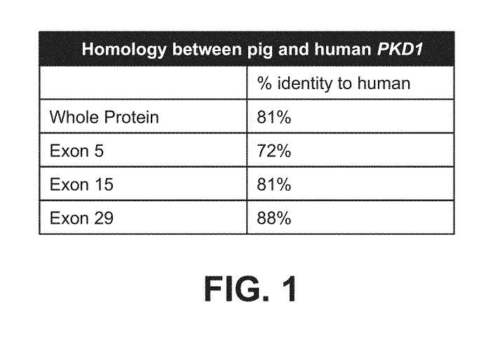

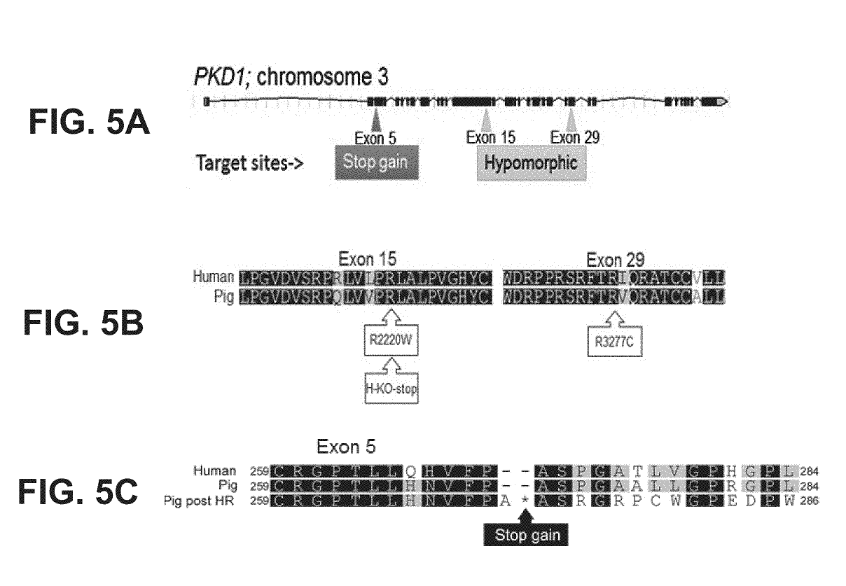

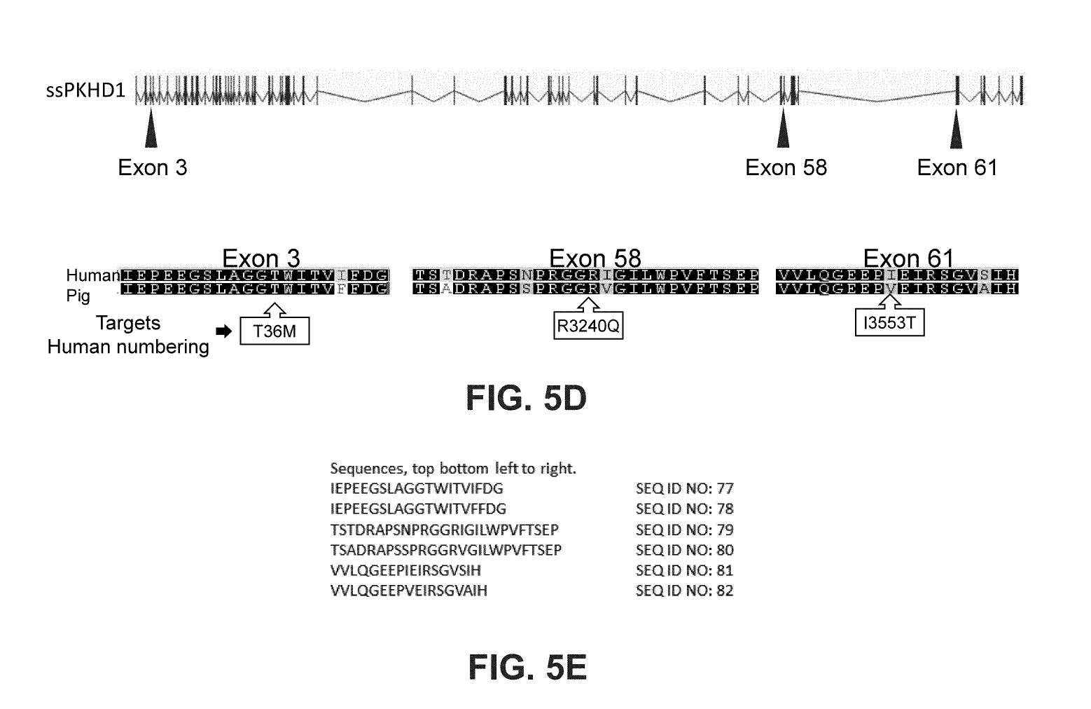

Presently, there is no approved treatment for ADPKD, but at least 12 potential therapies have been described as effective in non-orthologous or Pkd1 conditional or hypomorphic mouse models. However, results from human clinical trials for only three groups of compounds have been reported. One reason for the limited progression of potential treatments to clinical trials is that the mouse is a relatively poor model of human PKD. This is partly because mouse and human cystic physiologies are quite different. For instance, while mTOR inhibitors were effective in mouse models of Pkd1, they failed in human trials. This is largely because mice can tolerate a much higher dose of mTOR inhibitors than humans, without significant side effects. Another significant difference between humans and mice is that mice have much higher abilities to concentrate urine than humans. This is particularly important in ADPKD where antagonizing vasopressin stimulation pharmacologically or by encouraging drinking of large amounts of water may be advantageous. While blocking the V2 vasopressin receptor has been shown to be effective in humans, rats and mice, whereas water drinking was only of value in the PCK rat, is suspected to be helpful in humans, but is not effective in a mouse model of Pkd1. As outlined above, there is a great need for a larger animal model of ADPKD that better models human physiology of kidney disease. Such a model could be a significant breakthrough to facilitate the testing of therapies for this disorder (see letters of support). In this respect, it is noted that mutations associated with ADPKD in humans are also conserved in swine, therefore, mutation of these conserved residues to match those identified in humans should provide a better approximation of the disease in swine than in rodents, (FIG. 1, FIG. 5A, FIG. 5B, and FIG. 5C).

### ARPKD

Autosomal recessive (ARPKD) is a genetic disorder occurring in approximately 1 in 20,000 individuals. It is a devastating disease that strikes early, in fact up to 30% of ARPKD patients die as neonates. As many as 45% of those surviving the first month of life are prone to end stage renal disease, whereas those that live past 15 years old often succumb to liver disease. Current models in rats and mice are less than optimal, particularly for translation of treatment to the clinic; hence, there is a significant unmet need for a large animal model that better replicates human physiology to facilitate new treatment approaches. As disclosed, the present disclosure utilizes a viable pig model that manifests with the targeted morbidity while providing a lifespan conducive to research gains. In some embodiments, the ADPKD disclosures provided are readily applied to ARPKD where the genotype: phenotype relationship is somewhat stochastic, and a survey of multiple knockout or missense mutation phenotypes is required to establish models with predictable disease onset. Cytoplasmic injection of pig zygotes with gene-editing reagents targeting exon 3 of PKHD1 result in several combinations of ARPKD alleles from the most prevalent T36M allele to frame-shift and read-through indel alleles. This ad hock approach is used to produce pigs with a range of ARPKD severity from a single litter or produce the founder animals for breeding of the study population. Studies herein could be readily applied to other etiologies of renal failure, including ADPKD.

ARPKD presents in about 1 in 20,000 births and affects homozygous boys and girls equally. Due to the high and rapid penetrance, almost all patients are diagnosed during infancy or childhood. About 10% of children are diagnosed after 5 years of age due to either mild disease or the fact that screening is not part of standard clinical practice for newborns. The first signs of the disease vary greatly. Severe forms of the disease result in the “Potter” phenotype at birth, characterized by facial and skeletal abnormalities due to a lack of amniotic fluid provided by the fetal kidneys. Since amniotic fluid is also required for proper lung development, many of these infants die of respiratory insufficiency and pulmonary complications. The predominant clinical abnormalities in surviving infants are hypertension due to dysgenesis and hepatic fibrosis. Several newborn diseases become orphaned with very little drug or therapeutic development due to limited number of individuals and short lifespan to enroll in clinical trials. The most practical way to approach these newborn disorders is to develop animal models that can stand in place of the sick infants—a paradigm the FDA has accepted for other orphaned diseases.

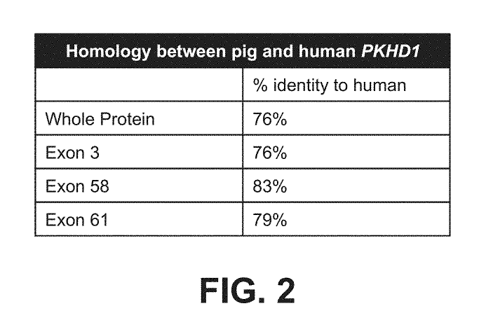

The genetic etiology of ARPKD was traced to mutations to the PKHD1 gene nearly 14 years ago (4). PKHD1 protein is strongly expressed in the kidney, with lower levels in the pancreas and liver (4, 5). PKHD1 is a large, (>450 kDa) membrane bound protein with somewhat elusive function, but its localization to the apical plasma membrane, primary cilium, mitotic spindle and basal body suggest cytoskeletal and fluid management functions along with speculated proliferation functions mediated by its similarity to hepatocyte growth factor receptor (3). A broad spectrum of disease alleles were identified, including splice site mutations, missense mutations and truncation mutations. Both alleles of PKHD1 are mutant in ARPKD patients, but they rarely (almost never) are homozygous for the same mutation. This diversity of disease alleles makes prediction of phenotype: genotype relationships very difficult, but it has been noted that variation in disease within family is less than that across families. The one consistency that can be noted for ARPKD is that less severe forms must have one or two missense alleles, and that the most severe forms are often caused by inheritance of two truncation mutants. Due to the diverse set of causative mutations and stochastic genotype: phenotype relationship, an empirical approach is necessary to hone in on the appropriate allele combination for early presentation of ARPKD, but sufficient survival to utilize the model for preclinical testing. However, residues conserved in humans and associated with ARPKD are also conserved in swine (FIG. 2, FIG. 5B, FIG. 5D, and FIG. 5E).

A number of rodent models of ARPKD that exist have been critical for the characterization of disease pathology and have resulted in valuable insights into the mechanisms of renal and hepatic cyst formation. However, mechanistically most of these spontaneous rat and mouse cystic disease models do not infer the same phenotype as their human orthologues. For example, mice carrying a homozygous pcy mutation were initially suggested as an ADPKD model due to their slow disease progression. However, sequencing analysis revealed that the mutated gene of this locus was actually the mouse orthologue of the human NPHP3 gene, responsible for human nephronphthisis. Similarly, in the rat, the rat orthologue of the PKHD1 gene, pck, responsible for ARPKD was originally thought to be an orthologue of ADPKD due to its slow disease progression. However, due to physiological differences, the disease characteristics have been shown to differ between rats and humans. For example, hepatic pathology of human ARPKD originates in the bile ducts progressing to the portal ducts causing them to become torturous, cystic, and encapsulated by variable degrees of fibrosis. Unlike with human disease, little evidence of cholestasis in the genetic homolog ARPKD rat model has been reported. The physiological differences between rats and humans may account for this outcome as rats lack a gallbladder and bile ducts are located superficially. Moreover, disease phenotypes of rodents can differ between strains with identical genotypes. For example, the original Sprague-Dawley (SD) rat pck mutation was introduced into the Fawn-Hooded Hypertensive (FHH) rat line. Phenotypical analysis revealed that although no differences in hepatic disease progression was observed, the FHH/pck rat showed a significant reduction in renal disease. It is recognized that, as of yet, unknown genetic factors play a role in the onset and severity of the disease and rodent models may be useful in identification of genetic modifier candidates. However, mounting evidence suggests that due to the phenotypic and physiological differences between rodent and human disease characteristics, another model species is required for confirmation of their contribution to disease progression.

In many respects, the anatomy, biochemistry, physiology, size, and genetics of pigs resemble those of humans, with pigs being a well-established model for cardiovascular and renal pathophysiology since the anatomy and physiology of the swine cardiovascular and urological systems are comparable to humans. Moreover, their body size affords the use of clinical imaging modalities for accurate monitoring of disease progression, particularly in the youngest patients. This is specifically critical for a newborn disease where the small size of neonatal rodents is not amenable to medical device employment or surgical interventions. In terms of abilities to concentrate urine, the pig also much closer matches the physiology in humans than do rodents and will help advance the ability of current and new researchers to define new data targets and therapies.

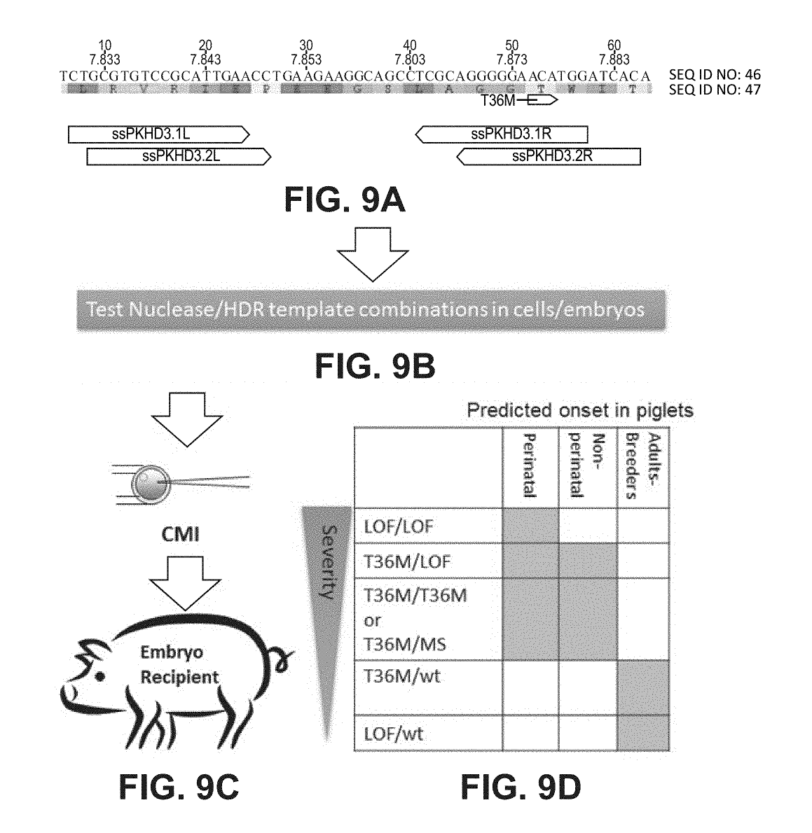

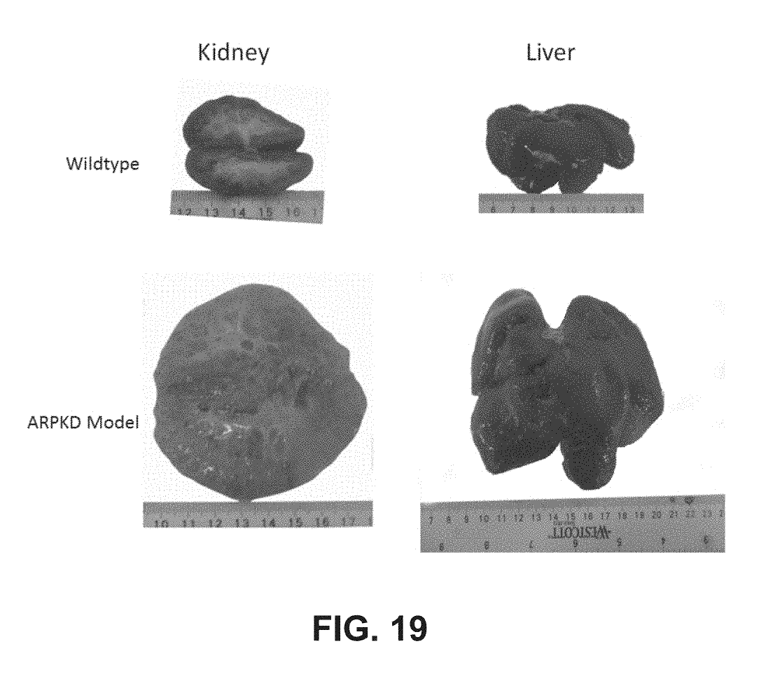

An animal model that can more closely replicate the complexity of how this disease actually presents in babies and children is sorely needed, particularly in the most severe neonatal presentations. To generate a useful model, a few predicted hypomorphic PKD alleles were combined with knockout alleles; and engineered cell lines were cloned to produce founders. The downfall of this approach is that it can only evaluate one set of alleles per cloned line of pigs—an extraordinary expense. In addition, cloning presents with a number of confounding consequences as epigenetic reprogramming in cloned pigs is a stochastic process and often incomplete (13, 14) leading to altered gene expression (15) and aberrant penetrance diseases such as x-linked SCID, dilated cardiomyopathy, and ADPKD ((16) and unpublished data). Consequently, an innovative approach enabling assessment of a variety of mutant genotypes of PKHD1 was identified. TALENs and CRISPRs operate by creating targeted DNA double strand breaks that can be repaired by either non-homologous end joining (NHEJ) or homology dependent repair (HDR) (17). Mutations from NHEJ often result in small insertion or deletion alleles (indels) that can lead to frame-shift truncation mutants, or in-frame mutation that may act as missense mutations. In addition, a template for the T36M mutation, the most commonly recovered mutation in ARPKD patients, is provided to direct a well-known missense mutation to PKHD1. Injection of the editing reagents into porcine zygotes produces pigs with a variety of mutant combinations enabling empirical assessment of genotype: phenotype relationships in pigs (FIG. 9A, FIG. 9B, FIG. 9C, FIG. 9D, FIG. 19 and Table 14)). Thus, this ad hock screening of allelic combinations provides the ability to produce unique models of ARPKD. Finally, this approach is more cost effective than cloning, and mitigates the risk of confounding epigenetic defects that may result from cloning, and results in higher production of newborn experimental animals. Use of direct zygote injections provides a viable a production method for monogenic, recessive newborn diseases that are difficult and costly to produce by cloning and breeding. Thus, disclosed are screening methods as well as model generation methods. Further, it should be noted that while, chimeras/mosaics are likely in a portion of offspring, in the case of PKD, the cysts are clonal so mosaic tissue that has some null cells and some wild type cell are still expected to develop cysts.

### The Pig as a Model of Polycystic Kidney Disease

Progress made since the development of a porcine model of cystic fibrosis illustrate how this species can greatly facilitate understanding of a human genetic diseases and the development of new therapies. In many respects such as: anatomy, biochemistry, physiology, size, and genetics, pigs more closely resemble humans than mice or rats. TABLE 1 provides a comparison between human/mouse and human/pig sequence homology of the entire PKD1 gene specifically between Exons 15 and 29.

TABLE 1

| | Full Gene | Exon 15 | Exon 29 |
|---|---|---|---|
| | Pairwise | Pairwise | Pairwise |
| Alignment | Identity (%) | Identity (%) | Identity (%) |
| Human/Mouse | 78.7 | 78.7 | 82.6 |
| Human/Pig | 81.5 | 80.7 | 88.4 |

Pigs are becoming well-established models for cardiovascular and renal pathophysiology since the anatomy and physiology of the swine cardiovascular and urological systems are comparable to humans. Moreover, their body size affords the use of clinical imaging modalities for accurate monitoring of disease progression. Importantly, in terms of assessing renal function, a key parameter for models tracking human kidney disease, the pig is much closer in terms of its ability to concentrate urine, TABLE 2 blood volume, 65 mg/kg; blood pressure 127/86; and heart rate, 105, than the mouse or the rat.

TABLE 2

| | Human | Pig | Mouse | Rat |
|---|---|---|---|---|
| Blood Volume | 79 ml/kg | 65 mg/kg | 55 ml/kg | 64 ml/kg |
| Blood Pressure | 120/80 | 127/86 | 120/71 | 129/91 |
| Heart Rate | 60-100 | 105 | 650-750 | 350-450 |
| Urine Conc. | 500-800 | 1000-2000 | 3,000-4,000 | 3,000-4,000 |
| mOsm/Kg | | | | |

These novel models of ADPKD more closely match human disease development and fill an important void present between identifying therapeutic targets and progression to clinical trials. For example, mice and rats are capable of more extreme urine concentrations in the range of 3,000-4,000 mOsm/kg, while humans and swine have a maximum capacity of about 1,200 mOsm/kg. This difference in kidney function makes difficult the ability of the clinician to assess the effects of PKD on kidney function when using mouse and rat models.

The ARPKD pig models developed and characterized herein are themselves an innovative product for the therapeutic industry. The disclosed pig models of ADPKD are the first available that develop significant disease within a timeframe consistent with both animal husbandry and pre-clinical testing. The pig is the ideal model to study human kidney disease because it is physically and physiologically similar, they breed easily with a relatively short gestation time (114 days), and do not pose ethical issues as does experimentation in non-human primates. This is reflected in the choice of pigs for developing kidney xenotransplantation. The disclosed approach of developing the model is also highly innovative. Information gained from the study of human families and the development of mouse models was used to introduce specific mutant alleles into the pig; thus, human genotypes serve as models for introduction of disease alleles into pigs. This knowledge is central to making a useful model since the disease course needs to be fairly rapid and progressive so that therapies can be tested in a short period (˜6 months) but not so aggressive that the pigs die at too young an age. Building on the ADPKD threshold and dosage dependence model of disease development and severity, the typical human disease course from cyst initiation to severe cystic disease, occurring over more than a quarter of a century, needs to be compressed into less than a year. This has been achieved by the innovative combinations of human PKD1 alleles to the level of functional PC1 to a low level (20-40%) compared to a 50% reduction typically seen with haploinsufficiency in adult-onset ADPKD.

In some embodiments, the methods of making a genetically edited pig disclosed herein comprise using a mini-pig. In some embodiments, the mini-pig is an Ossabaw, Yucatan, Bama Xiang Zhu, or Goettingen mini-pig. In some embodiments, the mini-pig is a Mulefoot, American, KuneKune, Juliana, Pot-bellied, or Choctaw mini-pig. In some embodiments, the methods of making a genetically edited pig disclosed herein comprise using a Yorkshire pig, a Landrace pig, a Duroc pig, or a Hampshire pig.

### Gene-Editing

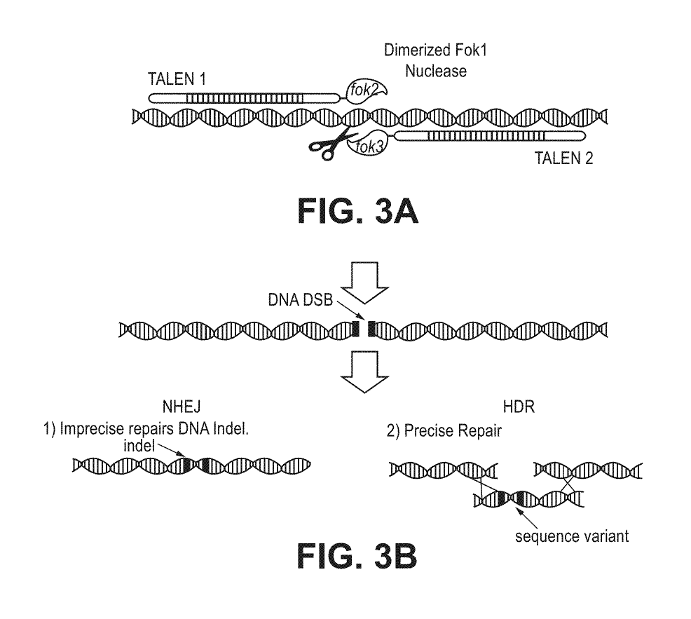

Novel methodologies for gene disruption and for generating specific base-pair changes have been developed within the last few years. Using these technologies, DNA editing to the resolution of a single base has been demonstrated. This resolution enables a most elegant approach to model human genetic disease in swine by precisely replicating human disease alleles without introducing extraneous sequences. These custom alleles are generated using nucleases such as TALEN, Zinc Fingers, Meganuclease or CRISPR/Cas9 stimulated homology directed repair (HDR) (FIG. 3A and FIG. 3B). Nucleases have been developed to knockout, knock-in, alter single nucleotides, or to simultaneously knockout several genes (multiplex gene-editing) in greater than 100 genes of pigs, cattle and goats (and preliminary data). Accordingly, gene editing was used to produce a battery of humanized PKD1 genotype combinations in pigs to deduce the combinations useful for modeling PKD in young pigs. Gene edited animals also provide a much better approximation of human disease states as they can replicate exact mutations found in humans to correlate to disease states. In addition, gene editing allows the editing or conversion of alleles in a genome without the introduction of transgenes which result in foreign DNA being introduced into a genome.

Modeling PKD with all-in-One Conditional Alleles

The ability to develop conditional knockouts has revolutionized basic and disease specific research in rodents, and for a number of years represented the most elegant mouse models of PKD. In addition, the course of disease can vary quite significantly based on when levels of PKD1 and/or PKD2 (ADPKD) and PKHD1 (ARPKD) transcripts are diminished, as noted above. Whereas the strong hypomorphic/null combinations discussed in Examples 1 and 2 have a fixed outcome due to low PC1 levels throughout development, the conditional model gives the option of pediatric or adult onset based on temporal activation. However, standard two-line strategy utilized in rodents, where one line carries a floxed allele and a second line expresses Cre or Flp recombinase from a tissues specific promoter, is not practical due to the excessive cost in development and maintenance of two lines and the 10-fold longer generation interval.

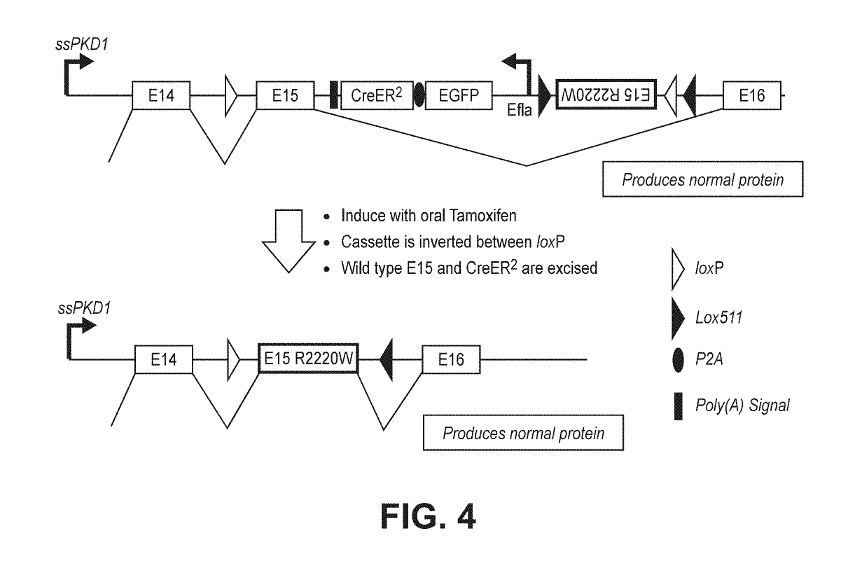

One disclosed innovative solution is to produce an all-in-one conditional allele to model ADPKD and ARPKD. Briefly, the conditional cassette consists of a wild-type exon preceding the mutant form of the exon facing the opposite direction (FIG. 4). The cassette is activated to cause disease by administration of tamoxifen. Tamoxifen induced CreER² recombination replaces the wild type exon 15 with the mutant R2220W version, for example for ADPKD or, for example the T36M version of PKHD1 for ARPKD. The cassette incorporates 3 innovative elements, 1) use of gene-editing to precisely introduce the cassette into the endogenous PKD1 gene, 2) use of tamoxifen inducible Cre for temporal control of disease induction and 3) a modified FLEx design that activates specific mutant allele of PKD1. This innovation enables replicating the human condition more closely where PC1 or PC2 or fibrocystin/polyductin (FPC) is not entirely abolished as typically done in conditional rodent models of ADPKD and ARPKD. In some embodiments, the methods of modeling ADPKD/ARPKD in pigs provided herein produce models that fulfill the needs of a wide variety of pre-clinical research. The models will revolutionize PKD research and fast-track treatment options because of much more rigorous preclinical testing. Such more rigorously tested compounds are less likely to fail during human studies with consequent improvements in patient options and reduced costs of clinical trials.

The development and characterization of the general and very specific edited pigs, as described here, are likely to revolutionize PKD research and to fast-track treatment options. This is underscored by the tremendous industry support for a PKD model that provides a more rigorous preclinical testing platform. State of the art genome engineering was utilized to develop the first swine PKD models with advanced disease. The clinical-grade characterization produces strong benchmark data required for characterization and utilization of the three novel models, (RC-null, RC-RC, and PKD1FLEX-CreER2), each expected to progress in severity at different rates. This well-characterized series of PKD models will fill the needs of a variety of preclinical testing designs and provide unique opportunities to evaluate prophylactic treatments of PKD. Of course, those of skill in the art will appreciate that, the all in one conditional cassette is not limited to PKD or the specific alleles referred to herein but can be utilized for observations of any allele phenotype that can be activated after birth or during pregnancy (i.e. in utero). In instances, it should be appreciated that the animal can also act as its own control with before activation comprising the wild-type phenotype (or other baseline phenotype) while post-activation provides an assessment of a heterozygous phenotype, compound heterozygote or hypomorph phenotype depending on the starting genotype or the number of inducible cassettes introduced into the zygote/embryo.

### Genetically Edited Animals

Animals may be edited using TALENs or other genetic engineering tools, including recombinase fusion proteins, or various vectors that are known. A genetic edit made by such tools may comprise disruption of a gene. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Embodiments of trans-acting gene are, e.g., genes that encode targeting endonucleases. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection, retrovirus mediated gene transfer into germ lines, gene targeting into embryonic stem cells, electroporation of embryos, sperm-mediated gene transfer, and in vitro transformation of somatic cells, such as cumulus or mammary cells, or adult, fetal, or embryonic stem cells, followed by nuclear transplantation. Pronuclear microinjection, sperm mediated gene transfer, and somatic cell nuclear transfer are particularly useful techniques. An animal that is genomically edited is an animal wherein all of its cells have the genetic edit, including its germ line cells. When methods are used that produce an animal that is mosaic in its genetic edit, the animals may be inbred and progeny that are genomically edited may be selected. Cloning, for instance, may be used to make a mosaic animal if its cells are edited at the blastocyst state, or genomic edit can take place when a single-cell is edited. Animals that are edited so they do not sexually mature can be homozygous or heterozygous for the edit, depending on the specific approach that is used. If a particular gene is inactivated by a knock out edit, homozygosity would normally be required. If a particular gene is inactivated by an RNA interference or dominant negative strategy, then heterozygosity is often adequate.

Typically, in pronuclear microinjection, a nucleic acid construct is introduced into a fertilized egg; 1 or 2 cell fertilized eggs are used as the pronuclear containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18-gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×10⁵ sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs can be injected into one of the pronuclei. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. For pigs, recipient females can be checked for pregnancy approximately 20-21 days after transfer of the embryos.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10-fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animals have been generated, expression of an exogenous nucleic acid can be assessed using standard techniques. Initial screening can be accomplished by Southern blot analysis to determine whether or not integration of the construct has taken place. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. Nucleic acids also can be amplified by ligase chain reaction, strand displacement amplification, self-sustained sequence replication, or nucleic acid sequence-based amplified. At the blastocyst stage, embryos can be individually processed for analysis by PCR, Southern hybridization, and splinkerette PCR.

Expression of a nucleic acid sequence encoding a polypeptide in the tissues of transgenic pigs can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) is known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically edited animal comprises inducing RNA interference against a target gene and/or nucleic acid such that expression of the target gene and/or nucleic acid is reduced. Add using RNAi for KO.

For example, the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Any of a variety of siRNA construct production methods, known to the skilled artisan, is applicable in the methods disclosed herein. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them is effective.

Embodiments include an in vitro cell, an in vivo cell, and a genetically edited animal such as a livestock animal that express an RNAi directed against a gene, e.g., a gene selective for a developmental stage. The RNAi may be, for instance, selected from the group consisting of siRNA, shRNA, dsRNA, RISC and miRNA.

### Inducible Systems

In some embodiments, the methods disclosed herein comprise the use of an inducible system. In some embodiments, the genetic edit is made using an inducible system. In some embodiments, the inducible system is a tetracycline-dependent regulatory system or a Cre/loxP recombinase system. In some embodiments, the inducible system comprises a conditional allele cassette. In some embodiments, the inducible system is induced by administering tamoxifen or tetracycline.

In some embodiments, the inducible system is induced when the pig or dog is in utero, about 1 day old, 2 days old, 3 days old, 1 week old, 2 weeks old, 3 weeks old, or 6 weeks old. In some embodiments, the inducible system is induced when the pig or dog is about 0.1 days to about 70 days. In some embodiments, the inducible system is induced when the pig or dog is at least about 0.1 days. In some embodiments, the inducible system is induced when the pig or dog is at most about 70 days. In some embodiments, the inducible system is induced when the pig or dog is about 0.1 days to about 1 day, about 0.1 days to about 2 days, about 0.1 days to about 3 days, about 0.1 days to about 5 days, about 0.1 days to about 7 days, about 0.1 days to about 14 days, about 0.1 days to about 21 days, about 0.1 days to about 42 days, about 0.1 days to about 49 days, about 0.1 days to about 56 days, about 0.1 days to about 70 days, about 1 day to about 2 days, about 1 day to about 3 days, about 1 day to about 5 days, about 1 day to about 7 days, about 1 day to about 14 days, about 1 day to about 21 days, about 1 day to about 42 days, about 1 day to about 49 days, about 1 day to about 56 days, about 1 day to about 70 days, about 2 days to about 3 days, about 2 days to about 5 days, about 2 days to about 7 days, about 2 days to about 14 days, about 2 days to about 21 days, about 2 days to about 42 days, about 2 days to about 49 days, about 2 days to about 56 days, about 2 days to about 70 days, about 3 days to about 5 days, about 3 days to about 7 days, about 3 days to about 14 days, about 3 days to about 21 days, about 3 days to about 42 days, about 3 days to about 49 days, about 3 days to about 56 days, about 3 days to about 70 days, about 5 days to about 7 days, about 5 days to about 14 days, about 5 days to about 21 days, about 5 days to about 42 days, about 5 days to about 49 days, about 5 days to about 56 days, about 5 days to about 70 days, about 7 days to about 14 days, about 7 days to about 21 days, about 7 days to about 42 days, about 7 days to about 49 days, about 7 days to about 56 days, about 7 days to about 70 days, about 14 days to about 21 days, about 14 days to about 42 days, about 14 days to about 49 days, about 14 days to about 56 days, about 14 days to about 70 days, about 21 days to about 42 days, about 21 days to about 49 days, about 21 days to about 56 days, about 21 days to about 70 days, about 42 days to about 49 days, about 42 days to about 56 days, about 42 days to about 70 days, about 49 days to about 56 days, about 49 days to about 70 days, or about 56 days to about 70 days. In some embodiments, the inducible system is induced when the pig or dog is about 0.1 days, about 1 day, about 2 days, about 3 days, about 5 days, about 7 days, about 14 days, about 21 days, about 42 days, about 49 days, about 56 days, or about 70 days.

In some embodiments, at least one phenotype is present at least about 6 weeks and older after induction of the inducible system. In some embodiments, at least one phenotype is present about 35 days to about 119 days. In some embodiments, at least one phenotype is present at least about 35 days. In some embodiments, at least one phenotype is present at most about 119 days. In some embodiments, at least one phenotype is present about 35 days to about 42 days, about 35 days to about 56 days, about 35 days to about 63 days, about 35 days to about 70 days, about 35 days to about 77 days, about 35 days to about 84 days, about 35 days to about 91 days, about 35 days to about 98 days, about 35 days to about 105 days, about 35 days to about 112 days, about 35 days to about 119 days, about 42 days to about 56 days, about 42 days to about 63 days, about 42 days to about 70 days, about 42 days to about 77 days, about 42 days to about 84 days, about 42 days to about 91 days, about 42 days to about 98 days, about 42 days to about 105 days, about 42 days to about 112 days, about 42 days to about 119 days, about 56 days to about 63 days, about 56 days to about 70 days, about 56 days to about 77 days, about 56 days to about 84 days, about 56 days to about 91 days, about 56 days to about 98 days, about 56 days to about 105 days, about 56 days to about 112 days, about 56 days to about 119 days, about 63 days to about 70 days, about 63 days to about 77 days, about 63 days to about 84 days, about 63 days to about 91 days, about 63 days to about 98 days, about 63 days to about 105 days, about 63 days to about 112 days, about 63 days to about 119 days, about 70 days to about 77 days, about 70 days to about 84 days, about 70 days to about 91 days, about 70 days to about 98 days, about 70 days to about 105 days, about 70 days to about 112 days, about 70 days to about 119 days, about 77 days to about 84 days, about 77 days to about 91 days, about 77 days to about 98 days, about 77 days to about 105 days, about 77 days to about 112 days, about 77 days to about 119 days, about 84 days to about 91 days, about 84 days to about 98 days, about 84 days to about 105 days, about 84 days to about 112 days, about 84 days to about 119 days, about 91 days to about 98 days, about 91 days to about 105 days, about 91 days to about 112 days, about 91 days to about 119 days, about 98 days to about 105 days, about 98 days to about 112 days, about 98 days to about 119 days, about 105 days to about 112 days, about 105 days to about 119 days, or about 112 days to about 119 days. In some embodiments, at least one phenotype is present about 35 days, about 42 days, about 56 days, about 63 days, about 70 days, about 77 days, about 84 days, about 91 days, about 98 days, about 105 days, about 112 days, or about 119 days.

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements.

An example of an inducible system is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

In some embodiments, the tetracycline-inducible system and the Cre/loxP recombinase system (either constitutive or inducible) are inducible systems used by the methods disclosed herein. The tetracycline-inducible system involves a tetracycline-controlled transactivator (tTA)/reverse tTA (rtTA). A method to use these systems in vivo involves generating two lines of genetically edited animals. One animal line expresses the activator (tTA, rtTA, or Cre recombinase) under the control of a selected promoter. Another set of transgenic animals express the acceptor, in which the expression of the gene of interest (or the gene to be edited) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a foxed stop codon is inserted between the promoter sequence and the DNA of interest. Genetically edited animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

### CreER2 (CreERT2)

CreER² or CreERT² refers to a Cre/lox site-specific recombination system. This system allows for the control gene activity in space and time in almost any tissue. The system provides a major technical advance in terms of in vivo inducibility providing a ligand-dependent Cre recombinases that can be activated by administration of tamoxifen to the animal. This temporal control is achieved by using a fusion between Cre and a mutated form of the ligand-binding domain of the estrogen receptor which preferentially binds tamoxifen (ERT and ERT2). This inactive Cre-ERT fusion is activated upon tamoxifen (or 4-hydroxytamoxifen), administration to induce recombination between loxP sites. Upon the introduction of tamoxifen (an estrogen receptor antagonist), the Cre-ERT construct is able to penetrate the nucleus and induce targeted mutation. ERt binds tamoxifen with greater affinity than endogenous estrogens, which allows Cre-ERt to remain cytoplasmic in animals untreated with tamoxifen. The temporal control of SSR activity by tamoxifen permits genetic changes to be induced later in embryogenesis and/or in adult tissues. This allows researchers to bypass embryonic lethality while still investigating the function of targeted genes.

Embodiments include an in vitro cell, an in vivo cell, and a genetically edited animal such as a livestock animal that comprise a gene under control of an inducible system. The genetic edit of an animal may be genomic or mosaic. The inducible system may be, for instance, selected from the group consisting of Tet-On, Tet-Off, Cre-lox, CreT², and Hiflalpha or the like. An embodiment is a gene set forth herein.

In some embodiments, a method of making a genetically edited pig or dog for use as a model for studying autosomal dominant polycystic kidney disease (ADPKD) disclosed herein comprises the use of a CreER² recombinase. In some embodiments, a method of making a genetically edited pig or dog for use as a model for studying autosomal recessive polycystic kidney disease (ARPKD) disclosed herein comprises the use of a CreER² recombinase. In some embodiments, the inducible system comprises a CreER² recombinase. In some embodiments, the conditional allele cassette comprises a CreER² recombinase. In some embodiments, the conditional allele cassette comprises a wild-type exon preceding a mutant form of the exon facing the opposite direction and a CreER² recombinase.

### Dominant Negatives

Genes may thus be disrupted not only by removal or RNAi suppression but also by creation/expression of a dominant negative variant of a protein which has inhibitory effects on the normal function of that gene product. The expression of a dominant negative (DN) gene can result in an altered phenotype, exerted by a) a titration effect; the DN PASSIVELY competes with an endogenous gene product for either a cooperative factor or the normal target of the endogenous gene without elaborating the same activity, b) a poison pill (or monkey wrench) effect wherein the dominant negative gene product ACTIVELY interferes with a process required for normal gene function, c) a feedback effect, wherein the DN ACTIVELY stimulates a negative regulator of the gene function.

### Founder Animals, Animal Lines, Traits, and Reproduction

Founder animals (F0 generation) may be produced by cloning and other methods described herein. The founders can be homozygous for a genetic edit, as in the case where a zygote or a primary cell undergoes a homozygous edit. Similarly, founders can also be made that are heterozygous. The founders may be genomically edited, meaning that the cells in their genome have undergone an edit. Founders can be mosaic for an edit, as may happen when vectors are introduced into one of a plurality of cells in an embryo, typically at a blastocyst stage. Progeny of mosaic animals may be tested to identify progeny that are genomically edited. An animal line is established when a pool of animals has been created that can be reproduced sexually or by assisted reproductive techniques, with heterogeneous or homozygous progeny consistently expressing the edit. Consequently, a “line” of animals or animal “line” refers to a genetically homogenous group of animals with known genetics.

In livestock, many alleles are known to be linked to various traits such as production traits, type traits, workability traits, and other functional traits. Artisans are accustomed to monitoring and quantifying these traits. An animal line may include a trait chosen from a trait in the group consisting of a production trait, a type trait, a workability trait, a fertility trait, a mothering trait, and a disease resistance trait. Further traits include expression of a recombinant gene product.

### Recombinases

Embodiments of the disclosure include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance, a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

### Compositions and Kits

The present disclosure also provides compositions and kits containing, for example, nucleic acid molecules encoding site-specific endonucleases, CRISPR, Cas9, ZNFs, TALENs, RecA-gal4 fusions, polypeptides of the same, compositions containing such nucleic acid molecules or polypeptides, or engineered cell lines. An HDR may also be provided that is effective for introgression of an indicated allele. Such items can be used, for example, as research tools, or therapeutically.

### Genetic Markers

Livestock may be genotyped to identify various genetic markers. Genotyping is a term that refers to the process of determining differences in the genetic make-up (genotype) of an individual by determining the individual's DNA sequence using a biological assay and comparing it to another individual's sequence or to a reference sequence. A genetic marker is a known DNA sequence, with a known location on a chromosome; they are consistently passed on through breeding, so they can be traced through a pedigree or phylogeny. Genetic markers can be a sequence comprising a plurality of bases, or a single nucleotide polymorphism (SNP) at a known location. The breed of a livestock animal can be readily established by evaluating its genetic markers. Many markers are known and there are many different measurement techniques that attempt to correlate the markers to traits of interest, or to establish a genetic value of an animal for purposes of future breeding or expected value.

### Homology Directed Repair (HDR)

Homology directed repair (HDR) is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations, but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences (otherwise known as homology arms) that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site. Of note, HDR is used only in dividing cells which NHEJ (below) is used in both dividing and non-dividing cells.

### Non-Homologous End Joining (NHEJ)

Non-Homologous End Joining (NHEJ) is a method of double strand break repair. In NHEJ the breaks are directly ligated without the need for a homologous template. NHEJ uses short homologous DNA sequences called microhomologies to guide repair. The microhomologies are often present in single-stranded overhangs. When the overhangs are compatible, NHEJ can repair the break accurately, however, when the overhangs are not compatible; repair can lead to the loss of nucleotides or translocations. As noted above, NHEJ is used by both dividing and non-dividing cells.

### Microhomology-Mediated End Joining (MMEJ)

Microhomology-Mediated End Joining (MMEJ) is a third pathway for repairing double stand breaks in DNA. MMEJ uses 5-25 bp or microhomologous sequences during the alignment of broken ends before joining, generally resulting in deletions flanking the original break. MMEJ is frequently associated with chromosome abnormalities such as deletions, translocations, inversions and other complex rearrangements.

### Single Strand Annealing (SSA)

Single strand annealing (SSA) is a process that is initiated when a double strand break is made between two repeated sequences oriented in the same direction. Single stranded regions are created adjacent to the break that extend to the repeated sequences such that the complementary strands can anneal to each other. This annealed intermediate can be processed by digesting away the single stranded tails and filling in the gaps. In some embodiments, this process can be used to insert large dsDNA cargo that is flanked by repeated sequences.

### Targeted Endonuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a RGEN. tracrRNA is another such tool. These are examples of targeted nuclease systems: these systems have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at or near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALs has been reported (PCT Publication WO 2011/072246) wherein each DNA binding repeat is responsible for recognizing one base pair in the target DNA sequence. The residues may be assembled to target a DNA sequence. In brief, a target site for binding of a TALEN is determined and a fusion molecule comprising a nuclease and a series of RVDs that recognize the target site is created. Upon binding, the nuclease cleaves the DNA so that cellular repair machinery can operate to make a genetic edit at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene edit in immortalized human cells by means of the two-major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindlII, NotI, BbvCl, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a targeted endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present disclosure. Examples of such endonuclease include I-See I, I-Chu L I-Cre I, I-Csm I, PI-See L PI-Tti L PI-Mtu I, I-Ceu I, I-See IL 1-See III, HO, PI-Civ I, PI-Ctr L PI-Aae I, PI-Bsu I, PI-Dha I, PI-Dra L PI May L PI-Meh I, PI-Mfu L PI-Mfl I, PI-Mga L PI-Mgo I, PI-Min L PI-Mka L PI-Mle I, PI-Mma I, PI-30 Msh L PI-Msm I, PI-Mth I, PI-Mtu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-MsoI.

A genetic edit made by TALENs or other tools may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified, and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting non-human vertebrates, non-human primates, cattle, horse, swine, sheep, chicken, avian, rabbit, goats, dog, cat, laboratory animal, and fish.

Some embodiments involve a composition or a method of making a genetically edited livestock and/or artiodactyl comprising introducing a TALEN-pair into livestock and/or an artiodactyl cell or embryo that makes a genetic edit to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically edited animals may be made from the embryos or cells according to known processes, e.g., implantation of the embryo into a gestational host, or various cloning methods. The phrase “a genetic edit to DNA of the cell at a site that is specifically bound by the TALEN”, or the like, means that the genetic edit is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic edit comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences, and this enables zinc-finger nucleases to target unique sequences within complex genomes. By taking advantage of endogenous DNA repair machinery; these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically edited animals are disclosed in, e.g., U.S. Pat. No. 8,106,255; U.S. 2012/0192298; U.S. 2011/0023159; and U.S. 2011/0281306.

Precise Integration into Target Chromosome (PITCh)

“PITCh,” as used herein, refers to Precise Integration into Target Chromosome. PITCh is a gene knock-in approach based on microhomology-mediated end-joining and or SSA- the exact mechanism not yet determined. In the PITCh system, the targeting vector and the genomic target site are simultaneously cut by TALENs or CRISPR (TAL-PITCh or CRISP-PITCh respectively), then the linearized DNA fragment is integrated into the genome via short microhomologies in the range of 8-72 bp. In some instances, generic single-guide RNA (sgRNA) are used to cleave the PITCh donor vector.

### Homology-Independent Targeted Integration (HITI)

HITI allows insertion of transgenes into both proliferating and non-proliferating cells. HITI targets an insertion site using CRISPR/Cas9, supplies an excess of linear DNA template, and allows the cells to insert the DNA template between the ends of the cut target DNA via NHEJ. If the cell anneals the two ends back together without the insert (or a mutation), the Cas9 target site would re-form and get cut again. Similarly, the designed donor DNA can be designed so that it also re-forms the cut site if it goes in backwards, ensuring that most insertions are the correct orientation. In addition, continued cleavage by Cas9 results in gRNA that is no longer able to bind to target sequences due to errors during NHEJ repair.

In some embodiments, the editing of the PKHD1 gene comprises use of homology-independent targeted integration (HITI) or precise integration into target chromosome (PITCh) technology. In some embodiments, the editing of the PKD1 gene comprises use of homology-independent targeted integration (HITI) or precise integration into target chromosome (PITCh) technology. In some embodiments, the editing of the PKD2 gene comprises use of homology-independent targeted integration (HITI) or precise integration into target chromosome (PITCh) technology. In some embodiments, any genetic edits done to any of the genetically edited animals (e.g., pigs or dogs) disclosed herein comprise the use of homology-independent targeted integration (HITI) or precise integration into target chromosome (PITCh) technology.

In some embodiments, the editing of the PKHD1 gene comprises use of homology-independent targeted integration (HITI) and precise integration into target chromosome (PITCh) technology. In some embodiments, the editing of the PKD1 gene comprises use of homology-independent targeted integration (HITI) and precise integration into target chromosome (PITCh) technology. In some embodiments, the editing of the PKD2 gene comprises use of homology-independent targeted integration (HITI) and precise integration into target chromosome (PITCh) technology. In some embodiments, any genetic edits done to any of the genetically edited animals (e.g., pigs or dogs) disclosed herein comprise the use of both homology-independent targeted integration (HITI) and precise integration into target chromosome (PITCh) technology.

### Vectors and Nucleic Acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be edited at the base moiety, sugar moiety, or phosphate backbone to improve, for example, stability, hybridization, or solubility of the nucleic acid. The deoxyribose phosphate backbone can be edited to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

In general, type of promoter can be operably linked to a target nucleic acid sequence. Examples of promoters include, without limitation, tissue-specific promoters, constitutive promoters, inducible promoters, and promoters responsive or unresponsive to a particular stimulus. In some embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable expressed markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty; Frog Prince; Tol2; Minos; Hsmar1; and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically edited organism or genetically engineered organism whose genetic material has been altered using genetic engineering techniques. A knockout artiodactyl is thus transgenic regardless of whether or not exogenous genes or nucleic acids are expressed in the animal or its progeny.

### Example 1

### Development of Progressively Severe Swine Models of PKD

Multiplex gene editing and cloning was used to create two lines of founders 1) homozygous for a hypomorphic allele of PKD1 (PDK1^{R2220W/R2220W}), and 2) compound heterozygotes with hypomorphic and loss of function alleles of PKD1 in trans (PDK1^{R2220W/e5null}).

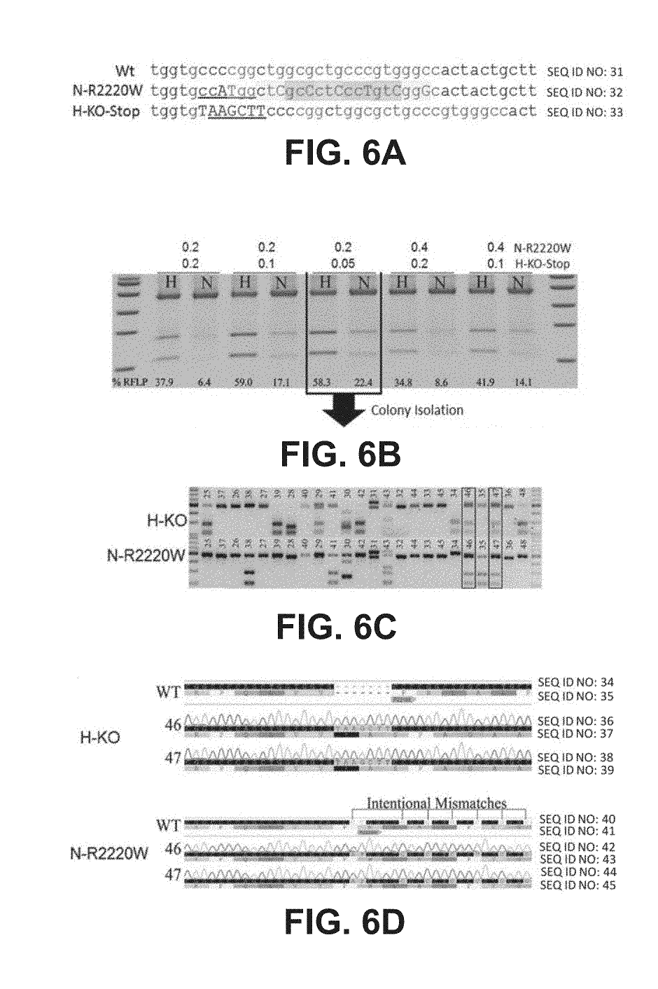

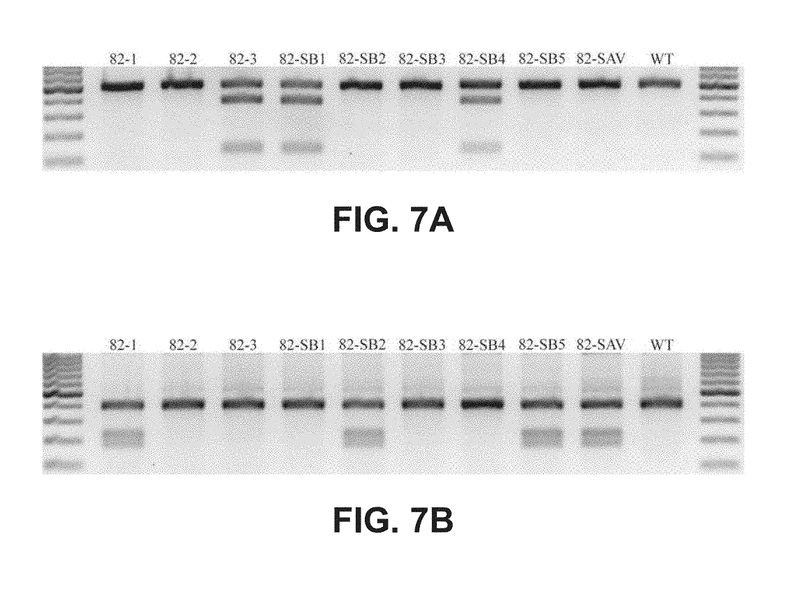

Gene edited fibroblasts were produced and validated for each genotype. To achieve this, nucleases (TALENs) were developed to cut in exons 5, 15 and 29 of the pig PKD1 gene with respective HDR templates to create a null, stop-gain allele in exon 5 and presumptive hypomorphic alleles PKD1:p.R2220W and PKD1:p.R3277C (FIG. 5A, FIG. 5B, FIG. 5C, FIG. 5D, and FIG. 5E). An additional template was also developed for exon 15 (H-KO-stop) that confers a pre-mature stop codon to produce a null allele (FIG. 5A, FIG. 5B, FIG. 5C, FIG. 6A, FIG. 6B, FIG. 6C, FIG. 6D and FIG. 4). A traditional approach to evaluate several combinations of these PKD1 alleles would require independent derivation of each line and a series of intercrosses to test each allelic combination; a costly and time consuming endeavor. In some embodiments, the animal models provided herein comprise a simultaneous introduction of both a null and hypomorphic allele into the swine PKD1 gene. The first combination developed was R2220W/−. To accomplish this in a single transfection, the exon 15 TALENs were co-transfected with both the N-R220W and H-KO-Stop HDR templates to confer the R2220W or null allele, respectively (FIG. 6A). TABLE 3 provides the TALEN RVD sequences used. In some embodiments, insertion templates (like H-KO-Stop) are much more effective for HDR than templates that confer only point mutations, (like N-R2220W). Insertion templates (like H-KO-Stop) were transfected at different molar ratios to optimize the efficiency of the desired outcome: to produce R2220W/− cells (TABLE 4 provides a listing of HDR templates used). The reaction was most efficient when the R2220W template was at a 4-fold excess over the null oligo (FIG. 6B), and these cells were used to derive individual colonies of fibroblasts. Restriction fragment length polymorphism assay (RFLP) was used to screen colonies for inclusion of one R2220W allele and one null allele (FIG. 6C). Amplicons from positive candidates were then sequenced to confirm the precise presence of both alleles (FIG. 6D). TABLE 5, provides a list of PCR primers used. This is the first example of intra-locus multiplex gene editing in any model organism. This innovative approach is capable of producing each desired PKD haplotype in a single generation, a unique aspect of this disclosure. This strategy was also used to produce the R3227C cells (FIG. 7A), previously identified as an alternative approach to R2220W animals. Finally, exon 5, heterozygous mutant cells were created to develop a breeding herd to propagate hypomorph/null offspring described in Example 2, below.

TABLE 3
TALEN RVD SEQUENCES

| | | Nucleotide Binding Sequence |
|---|---|---|
| Name | RVD Sequence | (5′-3′) |
| ssPKD1 5.1 L | HD HD NI HD NI NI HD NN NG HD | CCACAACGTCTTCCC |
| | NG NG HD HD HD | SEQ ID NO:2 |
| ssPKD1 5.1 R | HD NN NN NN NN HD HD HD HD | CGGGGCCCCAGCAGGGC |
| | NI NN HD NI NN NN NN HD | SEQ ID NO:4 |
| ssPKD1 15.1 L | NN NG NN NI NN HD HD NN NN | GTGAGCCGGCCCCAGC |
| | HD HD HD HD NI NN HD | SEQ ID NO:6 |
| ssPKD1 15.1 R | NN NN HD HD HD NI HD NN NN | GGCCCACGGGCAGCGCCA |
| | NN HD NI NN HD NN HD HD NI | SEQ ID NO:8 |
| ssPKD1 29.3 L | HD HD NI NG NN NG NN NN NN | CCATGTGGGACCGGCC |
| | NI HD HD NN NN HD HD | SEQ ID NO:10 |
| ssPKD1 29.3 R | NN NN HD HD HD NN HD NG NN | GGCCCGCTGGACGCGAGTG |
| | NN NI HD NN HD NN NI NN NG | SEQ ID NO:12 |
| | NN | |

TABLE 4
Repair Templates

| Template/ | |
|---|---|
| SEQ ID NO: | HDR TEMPLATE |
| ssPKD1 15.1 | cgccccagccgcccggctcggctgaccctgcccggcgtggaCgtgTCcTggccccagctggtggt |
| 2213 R to W | gccccggctggcgctgcccgtgggc |
| SEQ ID NO: 13 | Capital letters = SNPs, Italic = R2213W, Underline = AflIII site |
| ssPKD1 15.1 | accctgcccggcgtggatgtgagccggccccagctggtggtgTAAGCTTccccggctggcgctg |
| HR-KO | cccgtgggccactactgctttgtgtt |
| SEQ ID NO: 14 | Capital letters = Insertions, Italic = Stop codon, Underline = |
| | HindIII site |
| ssPKD1 15.1 | cggctcggctgaccctgcccggcgtggatgtgTCTAgAccTcagctggtggtgcccTggctggc |
| R2220W XbaI | gctgcccgtgggccactactgctttg |
| SEQ ID NO: 15 | Capital letters = SNPs, Italic = R2220W, Underline = XbaI site |
| ssPKD1 15.1 | gcccggctcggctgaccctgcccggcgtggatgtCTCGAggccTcaATtggtggtgccccggc |
| WT-XhoI | tggcgctgcccgtgggccactactgct |
| SEQ ID NO: 16 | Capital letters = SNPs, Underline = XhoI site |
| ssPKD1 29.3 | gctctccatgtgggaccggccgcctcggagccgcttcacATgTgtccagAgAgccacctgctgtg |
| R to C | ccctcctcctctgcctcttcctggg |
| SEQ ID NO: 17 | Capital letters = SNPs, Italic = R3277C, Underline = AflIII Site |
| ssPKD1 5.1 HR- | cctgcagggggcccaccctcctccacaacgtatccctgcctAAGcTTcccgggggcggccctgct |
| KO | ggggccccgaggacccctggcctc |
| SEQ ID NO: 18 | Capital letters =Insertions, Italic = Stop codon, Underline = |
| | HindIII site |
| ssPKD1 | ggcgtggatgtgagccggccccagctggtggtgccATggctCgcCctCccTgtCggGcactact |
| R2220W SEQ | gctttgtgttcgtggtgtcatttggc |
| ID NO: 19 | Capital letters indicate SNPs, Italic = R2220W, Underline = |
| | NcoI Site |

TABLE 5
Primers

| Name | Sequence (5′-3′) | SEQ ID NO: |
|---|---|---|
| ssPKD1 E5 NJ F1 | CCTCGCAGCCCTCTCAGA | SEQ ID NO: 20 |
| ssPKD1 E5 NJ R1 | CACCGAGGCCAGACACAC | SEQ ID NO: 21 |
| ssPKD1 E15 NJ F2 | TCCTACCAGACCGAGTACCG | SEQ ID NO: 22 |
| ssPKD1 E15 NJ R2 | GCTGAGATCAGAGCCACCAG | SEQ ID NO: 23 |
| ssPKD1 E29 NJ F1 | TGGTGGAGAAGGAGGTGCT | SEQ ID NO: 24 |
| ssPKD1 E29 NJ R1 | GAAGGATCAGGCTGGGCTG | SEQ ID NO: 25 |

### Example 2

### Production of Founders for Each Genotype

The R2220W edited cell lines were cloned by SCNT, resulting in 3 pregnancies diagnosed at 30 days post transfer (Table 6). By day 60, two pregnancies had aborted and by day 100 (of 114) there were no signs of pregnancy in the last recipient. Other cell lines cloned at the same time had a pregnancy and farrowing rate of 75% (n=8) and produced 22 gene edited offspring (data not shown), therefore, it was suspected that the R2220W/− conferred too strong of a phenotype to permit full-term survival of cloned piglets.

TABLE 6
Summary of founder production by genotype

| Genotype | Surrogates | Pregnant | Full term | Piglets |
|---|---|---|---|---|
| R2220W/− | 4 | 3 (75%) | 0 | 0 |
| RC/null | 3 | 1 (33%) | 1 | 1 |
| RC/+ | 3 | 1 (33%) | 1 | 2 |
| +/−* | 3 | 2 (66%) | 2 | 5 |

The “*” allele in these pigs carry the ssPKD1 5.1HR-KO shown in Table 4

A weaker hypomorphic allele, R3277C (RC), was identified, and these cells (as shown in FIG. 7B) were produced. First RC/null cells were cloned resulting in one pregnancy and a single piglet. The piglet had an incomplete closure of the abdomen at the umbilical entry site, was euthanized and is described in detail below. One RC/+ pregnancy also went full term giving rise to two healthy gilts that are now at breeding age. In addition to the hypomorphic combinations, PKD1^{+/-} females were cloned to enable breeding to produce RC/null offspring. A total of 5 animals were born live, 3 of which have now produced two litters and transmitted the KO allele to offspring. Based on serum biochemistry, the RC/+ and +/− animals have normal kidney function, as expected (data not shown). Hence, breeding herds have been established to translate from prototype to production. To produce PKD1^{R3277C/-} piglets, intercross is performed between PKD1+/− and PKD1+/R3277C animals (FIG. 7A and FIG. 7B). Intercross between pigs with PKD1+/R3277C genotypes are used to produce PKD1^{R3277C/R3277C} swine.

### Example 3

Serial Monitoring of Renal Function and Kidney Morphology in PKD1^{R3277C/-} or PKD1^{R3277C/R3277C} Swine

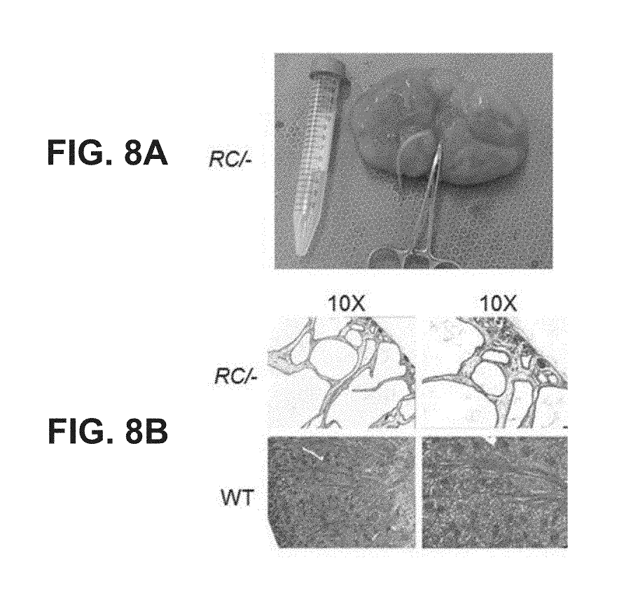

After the RC/null piglets were euthanized, gross necropsy revealed highly cystic kidneys with >10× increase in volume versus wild type newborn pigs (FIG. 8A). Pathological analysis confirmed the advanced PKD phenotype wherein the majority of the kidney structure was cystic (FIG. 8B). Blood and urine were not available from this animal. A second piglet from the PKD1^{+/-} failed to thrive and was euthanized at 4 days of age. Unexpectedly, the kidneys of this animal were highly cystic and BUN levels were elevated (data not shown). The genotype on this animal was confirmed to be PKD1^{+/-} and based on comparison with literature, was expected to be asymptomatic to at least 18 months of age. It is suspected that the stochastic epigenetic reprogramming during development cause preferential expression of the KO allele, thereby reducing the levels of PC1 and rapidly accelerating disease. The analogous effect has been observed in IL2Rg knockout females produced by cloning where heterozygotes unexpectedly were immunodeficient. The remaining 4 animals are 1.5-years-old and do not show clinical sign of disease. Again, it is suspected that due to deficiencies in epigenetic reprogramming leading to altered gene expression and higher morbidity of clones versus breed animals, the phenotype in both RC/− and R2220W/− animals could be exacerbated. This was observed with the advanced phenotype in the 4-day old PKD1^{+/-} animal and the RBM20^{R636S/R636S} models of dilated cardiomyopathy where all homozygous clones died within 1 day (n=10) while homozygotes can live past 3 months of age when produced by breeding (n>20).

### Example 4

### Production of Two Models of PKD by Breeding and Evaluation of Neonatal Outcomes

RC-RC and RC-null piglets (PDK1^{R3277C/R3277C}, PDK1^{R3277C}) are produced by intercross (FIG. 7B) and evaluated against the following performance parameters. Proper inventory management decisions on production of product animals, RC-RC and RC-null genotypes, are used to determine the production rate of the animals from birth to the saleable age. These experiments allow for the determination if production is in fact Mendelian, or if reduced fitness of the product animals would result in a lower production rate than expected. Finally, these experiments provide phenotypic benchmarks.

### A: Achieve Mendelian Distribution of Expected Genotypes

The established breeding herd consists of PKD^{+/-}, and RC/+ animals sufficient to produce 9 litters each of intercross between PKD^{+/-} RC/+ and RC/+×RC/+ within about one year. However, considering a minimum of 9 litters with an expected litter size of 11, about 100 piglets is produced for each intercross. With a sample size of 100, 25 are expected to be RC/− or RC/RC, depending on the cross. To be within Mendelian predictions with a sample size of 100, 2 degrees of freedom, a total of 16 to 33 RC/− or RC/RC animals should be produced. FIG. 7A and FIG. 7B show initial crossbreed genotypes.

### B: Cysts Observed in Newborn RC-RC and RC-Null Animals

Newborn founders are monitored 24-48 hours after birth by ultrasonography and blood/urine chemistry to monitor cyst development and functional impairment, respectively. For any non-viable animals, necropsy and histopathology is performed for comparative analysis between humans with PKD and the disclosed pig models.

### C: Monitoring of Cyst Development by Ultrasound Analysis

The phenotypes in the two swine genotypes made in this study, RC-RC and RC-null, is assayed along with littermate controls. The kidneys of at least 14 newborn RC-RC, RC-null, and littermate control animals, n=7 each per sex is observed. Briefly, the kidneys are assayed by abdominal ultrasound using the SonoSite Vet M-Turbo with a convex 5-2 MHZ transducer 24-48 hours after birth. This analysis is used to quantify the total number of cysts found in each kidney per animal for each genotype. Cysts will need to be at least 5 mm in diameter to be recorded as cysts. In addition, the volume of each kidney is determined and the total kidney volume (TKV), including both kidneys, is recorded. The TKV is determined using length, width and depth measures and employing the ellipsoid formula.

### D: Monitoring of Renal Function in Newborns by Blood and Urine Analysis

A 2 ml venous blood sample is collected at 24-48 hours and again at 4 weeks of age in EDTA tubes. A urine collection of 50 ml is obtained at the same time using a supra-pubic catheter. Measurements of blood and urine chemistries are performed each month on the samples collected employing the pHOx Ultra Comprehensive Critical Care Analyzer (Nova Biomedical). Creatinine levels are determined both from plasma and urine and blood urea nitrogen (BUN) will also be measured. A full range of other blood chemistries is measured as necessary. Plasma and urinary protein (microalbumin) will also be measured by conventional sandwich ELISA (Bethyl Laboratories, E101-110, pigs). Serum creatinine levels in excess of 8 mg/dl would be an indication of severe uremia and that the pig would need to be euthanized.

### E. Results, and Alternative Embodiments

Mendelian production of both RC-RC and RC-null genotypes will occur; any deviation from this rate would likely be due to reduced fitness. Lack of fitness is a possibility based on the potency in the preliminary data where the RC-null animal had highly cystic kidneys at birth. However, as noted above, this single animal is not sufficient to evaluate the true penetrance in phenotype, and cloning can have stochastic effects on gene expression that can confound results. As in rodents and humans, it is expected that the RC-RC genotype is much less severe than RC-null model, which is expected to be born with a moderate to severe cystic phenotype.

### Example 5

### Benchmark Disease Progression in RC-RC and RC-Null Offspring Using Clinical-Grade Modalities.

Clinical-grade imaging, blood/urine tests, endpoint necropsy, and histopathology are used to monitor renal function and kidney morphology to benchmark against the human condition. These experiments characterize the disease progression and provide the benchmarked data for the kinetics of disease development of the RC/RC and RC-null models produced in Examples 3 and 4. The study enrolls a total of 21 animals of either sex, n=7 each for RC-RC, RC-null and wild type pigs starting at 30 days of age and monitored to 6 months of age by serial imaging and serum analysis.

### A. Detectable Reduction in Renal Function by 6 Months

Blood is collected once monthly and analyzed for BUN and cAMP levels, followed by kidney function and electrolyte analysis at study summation.

BUN/cAMP measurements: Briefly, blood plasma is separated (6,000 g, 15 minutes at 4° C.) from blood collected by venous puncture and used for the BUN assay (BUN-Urea, BioAssay Systems), and cAMP assay (Direct cAMP EIA Kit, ENZO). Both assays are performed following the manufacturer's protocol. At the summation of the study cAMP levels are measured from 100 to 200 mg of flash-frozen kidney. BUN levels are also measured from plasma or serum using the blood-gas pHOx-Ultra analyzer available through the Mayo Clinic Translational Polycystic Kidney Disease (PKD) Center, for example.

### B. Kidney Function and Electrolyte Analysis

The animals are placed in metabolic cages to measure the urine output prior sacrificing. At sacrifice, the animals are weighed and anesthetized with ketamine and xylazine. Blood is obtained by venous puncture for determination of plasma copeptin, creatinine, BUN, and electrolyte levels. Small representative parts of the right kidney and liver are fixed with 10% formaldehyde in phosphate buffer (pH 7.4). The tissues embedded in paraffin for histological studies. Parts of the left kidney, other half of the right kidney, liver and heart are immediately frozen in liquid nitrogen for determination of cAMP. Comparisons between the groups are performed by analysis of variance.

### C. Detectable Reduction in Renal Blood Flow by MRI by 6 Months

Each pig is scanned at three time-points, 6 weeks, 4 months and 6 months of age. The following parameters are characterized.

Magnetic Resonance (MR) Imaging Protocol: All MR acquisitions are acquired on a GE 3T scanner (GE Medical Systems, Discovery MR750w) in the supine position utilizing a multichannel surface coil. Anesthesia of the animals is maintained with inhalation of 1-2% isoflurane. No intravenous contrast is used. The sequences included in the imaging protocol are standard SSFSE axial, coronal and sagittal scout images followed by T1 (coronal lava sequence, with 10° flip angle, and reconstructed voxel resolution in plane of ˜1 mm, and slice thickness of 3 mm) and T2-weighted (coronal fast spin echo, TE=100 ms, TR=min, with reconstructed voxel resolution in plane of ˜1 mm, and slice thickness of 3 mm).

Following coronal and oblique coronal Fast Imaging Employing Steady-state Acquisition (FIESTA) localizing images of the renal arteries, cine phase contrast acquisitions are obtained for assessment of renal blood flow (RBF). Images are obtained perpendicular to each renal artery utilizing a velocity encoding (VENC) value of 100 cm/sec.

Measurement of Renal Blood Flow: Renal blood flow (RBF) is measured from the 2D phase contrast images on an AW workstation. A trained imaging analyst performs the RBF measurements. For each renal artery, measurements are performed three times and the average value for each artery is used. Region-of-interest (ROIs) is drawn at the border of the renal artery. In the case of multiple renal arteries feeding a single kidney, each artery is measured separately, and the sum of the measurements is used as the total RBF perfusion of that particular kidney.

### D. Quantitative Characterization of Kidney Size and Architecture by MRI and Histopathology

The MR images acquired at 6 weeks, 4 months and 6 months of age is analyzed to characterize organ volume, architecture, and texture using a combination of the standard and novel analysis techniques described above. Animals are sacrificed at 6 months of age for thorough histological and protein analysis.

Measurement of Organ Volume: kidney and liver segmentations are performed semi-automatically utilizing the MIROS software package. The software package has an interactive viewer that allows visualization of the image data in coronal, sagittal, and axial planes. Segmentations can be overlaid and edited with a range of interactive tools. Measurements are performed on the T2-weighted images. Total kidney volume (TKV) and total liver volume (TLV) is measured from these segmentations.

Assessment of Kidney Architecture: In order to take advantage of the wealth of information provided by the anatomical MR images, the T1 and T2-weighted images are used in conjunction with advanced image processing techniques to characterize cyst volume, number, and size distributions, as well as perform image texture feature analysis. Texture analysis has been applied and has been shown to predict subsequent renal function decline. This technique informs on structural differences between kidneys and is used to track renal tissue changes occurring.

Histological analysis of kidney and liver: At 6 months, pigs except those chosen for breeding, are euthanized and representative kidney and liver tissue taken for histological analysis. Renal tissue is cut from the excised kidney in the area of the hilum including both cortex and medulla. Both the renal and liver tissue is fixed in 10% neutral buffered formalin, dehydrated, and embedded in paraffin, per standard techniques. As necessary, renal tissue, adjacent to the formalin tissue, is flash frozen and embedded in OCT medium for frozen sections and histologic sections are prepared. Representative formalin fixed sections are stained with H&E and Masson's trichrome. Additional stains may also be employed, such as PAS and Sirius red. To determine the section of the nephron that is cystic, immunofluorescence markers for each tubule segment (lotus tetragonolobus agglutinin [LTA]—proximal tubule [PT], peanut agglutinin [PNA]—distal tubule, Tamm-Horsfall glycoprotein [THP]—loop of Henle, and Dolichos biflorus agglutinin [DBA] or aquaporin 2 [AQP2]—collecting duct [CD]) is examined. Cystic areas and fibrotic areas are calculated as previously described using three cross-sections per kidney and the MetaMorph software. In short, the cystic area is calculated by measuring the percent cystic area of the three cross-sections, adjusted to kidney area. Fibrotic volume is calculated from the percent fibrotic area of cortical images and adjusted to kidney area.

Analysis of PC1 in urinary microvesicles: PC1 forms two glycoforms with only the mature membrane form in urinary microvesicles. The animals are placed in metabolic cages to measure urine output prior to sacrificing to perform urinary microvesicle isolation and analysis of renal clearance. Animals are placed in metabolic cages with sufficient water and urine collection lasting approximately 12 hours. Urinary microvesicles are isolated by ultracentrifugation and western blotted from the final urine sample taken at 6 months, and from seven wildtype pigs. The western blot is analyzed with the N-terminal PC1 antibody 7e12 and the urinary microvesicle control antibody to PDCD6IP. PC1 is measured using the LICOR system (Odyssey Sa) to assure linear quantification of the signal intensity and compared between the wildtype and seven RC-null and seven RC-RC pigs.

Based on data already developed, the RC-null model will have moderate to severe disease with cystic and renal enlargement, evident by 1 month. The progress of the disease in this model is uncertain but could range from continued cystic development and enlargement over the 5-month period to very rapid cystic expansion followed by a decrease in TKV and premature lethality. Based on the human and mouse data the RC-RC model is expected to be more slowly progressive and viable with continued cystic expansion to 6 months. In both models, it is expected that cysts of various sizes will develop in both the cortex and medulla, with some exceeding 2 cm in diameter. The predominant site of the cysts is expected to be the CD, the major site of cyst development in human adult ADPKD. The 6-month time-point will allow integration of the imaging and renal function data with histological data and should lead to quantitative characterization of kidney size and architecture as described in part ‘D’, above. The combination of these analyses will lead to a comprehensive dataset critical to both characterization of the models and development of preclinical research protocols with the animals. Although the PKD1: p.R3277C allele was carefully selected, it is possible that neither of the genetic combinations will fully meet the needs of all preclinical research; the RC-null pigs could be too severe and the RC-RC pigs too mild. Therefore, to more fully characterize the disease progression, an inducible model system is developed as provided in Examples 6 and 7.

### Example 6

### Development of a Novel-Recombination Based Inducible PKD Cassette

The RC-RC and RC-null pigs are expected to be born with a moderate to severe cystic phenotype in the kidney, missing the opportunity to test drugs from the earliest stage of disease. The novel inducible model will fill two needs, 1) allow drug testing in the earliest stages of disease and 2) serve as alternative to the RC-RC and RC-null animals should their disease course be too rapid.

### A. Successful Production of an all-in-One Inducible PKD Cassette (PKD1^{FLEX-CreER2})

The sequence of the PKD1^{FLEX-CreER2}, described in FIG. 4, was assembled in silico. The cassette is intended to be activated post-natal, so the mutation that had the strongest effect in preliminary data, R2220W, was chosen. This choice also expands the available alleles for preclinical testing rather than duplicating R3277C allele used in germline edited swine of EXAMPLE 4 and EXAMPLE 5. Since this cassette must efficiently splice with the endogenous gene as illustrated in FIG. 4, potential alternative splice acceptors and donors within the cassette were predicted in silico and eliminated from the final design. The entire cassette is commercially synthesized. Both PITCh and HITI techniques have been successfully used by to target transgenes into the porcine ROSA locus at efficiencies greater than standard double-strand break induced homologous recombination in the Recombinetics lab. Hence, the cassette is flanked with HITI or PITCh compatible sequences for efficient integration into the swine PKD1 gene.

### B. Achieve >80% Activation of PKD1^{FLEX-CreER2} by Tamoxifen Administration In Vitro

Testing of PKD1^{FLEX-CreER2} is conducted using the immortalized pig kidney cell line, LLC-PK1. Prior to conducting studies with the cassette integrated into cells, the efficacy of Cre recombination is optimized in transient transfections +/− tamoxifen and measure recombination by qPCR. Should recombination be inefficient, alternative loxP sites, lox5171 or lox2272, is substituted in place of lox511 or alternative versions of the EF1α-EGFP-P2A-CreER² is tested.

### Development of the LLC-PK1, PKD1^{FLEX-CreER2} Targeted Cells for In Vitro Testing:

The ideal system for testing of the conditional cassette is within a porcine cell line that both expresses PKD1 and has PKD1^{FLEX-CreER2} stably integrated within the gene. Development of this resource is conducted in a two-stage process. First, the PKD1^{FLEX-CreER2} is integrated into the PKD1 gene using PITCh or HITI as described above. Briefly, a two TALEN approach is used where native exon 15 is flanked such that it is eliminated in the correctly targeted allele. EFPG positive clones are scanned followed by junction PCRs and long-range PCR/sequencing across the entire locus to confirm proper targeting and fidelity. Clones with heterozygous targeting are selected to measure the effect on a single allele. Secondly, since PKD1 isn't normally expressed in LLC-PK1 cells⁸¹, its expression by stable introduction of a tripartite activator, VP64-p65-Rta (VPR), fused to nuclease-null Cas9 is insured. The VPR cassette is incorporated into a Sleeping Beauty transposon along with gRNA's targeted to the promoter region of the PKD1 gene. Sleeping Beauty transposition is then used to randomly insert the VPR into the PKD1^{FLEX-CreER2} LLC-PK1 cells to activate the transcription of the native PKD1 gene. Activation of PKD1 expression is confirmed by qRT-PCR and western blotting. Additional quality control steps for antibodies and qPCR is performed.

In vitro testing in the LLC-PK1, PKD1^{FLEX-CreER2} targeted cells: The model cells developed above are treated in triplicate with 3 dosages of tamoxifen, and compared to non-treated cells. Percent recombination of the PKD1^{FLEX-CreER2} cassette is analyzed visually for the loss of EGFP using FACS and molecularly by quantitative PCR for molecular quantification of recombination. Splicing of the PKD1^{FLEX-CreER2} cassette is analyzed before and after recombination by RT-PCR from exon 14 to exon 16, followed by Sanger sequencing and qRT-PCR using primers spanning exon junctions of E14-15 and E15-16 respectively. Finally, induced and non-induced populations are compared at the protein level by western blotting.

### Results and Alternative Embodiments

Given the large size of exon 15, (3,620 bp) the final cassette is quite large and potentially prone to recombination due to two large homologous segments. When this proves cumbersome in vitro, the design is altered for a conditional version of R3277C located in the much smaller exon 29, (211 vs 3,620 bp). R2220W, activation of PKD1^{FLEX-CreER}2 requires successful HITI/PITCh to integrate the cassette by replacement of native exon 15. This is different than standard HITI or PITCh where transgenes are integrated into a single cut site. Hence, while targeting efficiency can differ based on locus, this challenge is overcome. In some embodiments, a CRISPR/Cas9 system is used. A second challenge is driving native expression of PKD1 in LLC-PK1 cells. For this a CRISPR/Cas9 guided tripartite activator (VPR) is used to activate expression. While it is preferable to first engineer the cells with the PKD1 targeted VPR, the constitutive expression of gRNAs to the promoter region would result in NHEJ at this site should CRISPR/Cas9 be used for HITI or PITCh integration. Effective TALENs results in strong PKD1 expression in cells with the integrated cassette. In instances where such expression is lacking aberrant splicing of the cassette is investigated using exon spanning RT-PCR and eliminated in revised versions of the cassette. Given the prior optimization of recombination in vitro, it is expected that >80% activation is achieved after tamoxifen treatment of the integrated cassette. When the readout of mRNA abundance or PC1 content by western blotting is not satisfactory, PC2 is further expressed in cells to enable cell localization studies of the wild type vs R2220W PC1 protein⁸¹. Similar localization studies are routinely conducted in the Harris laboratory.

### Example 7

### Production and Validationation of PKD1^{FLEX-CREER2} Founders

The PKD1^{FLEX-creER2} pig is developed by gene-targeting and cloning followed by initial characterization +/− Tamoxifen treatment. Inducible ADPKD models have been useful in rodents and afford maximum flexibility in disease state prior to pre-clinical studies versus conventional edited models. However, mouse models completely ablated expression of PC1, reducing the utility of the model. The innovative FLEX design overcomes this challenge, and enables production of an all-in-one cassette that is activated by a ubiquitous promoter rather than a tissue specific promoter; however, a tissue specific promoter (such as PKDH1) could be used to more precisely restrict PKD inactivation. Another advantage of various embodiments of this model is its construction in a miniature strain of pigs that, unlike conventional swine, will not outgrow clinical imaging equipment, enabling long-term studies.

### A. Knock-in of PKD1^{FLEX-CreER2} into Miniature Swine Fibroblasts for Cloning

The PKD1^{FLEX-CreER2} cassette is integrated into male and female, miniature swine fibroblasts using HITI or PITCh techniques established as described above in Example 6. Briefly, fibroblasts, TALEN mRNA and the PKD1^{FLEX-CreER2} cassette is co-transfected using the Neon Transfection system (Life Technologies) as previously described. Individual colonies are isolated by dilution cloning prior to junction analysis by PCR and Sanger sequencing to confirm integrity of the cassette and correct targeting. Clones that are homozygous for integration, heterozygous for integration, and compound heterozygotes, where one allele carries the PKD1^{FLEX-CreER2} and the second allele is a knockout via deletion of exon 15, are recovered. In some embodiments, founder production with the compound heterozygote lines proceed as they should be equivalent to PKD1^{+/-} pigs where the phenotype is mild. Hence, compound heterozygotes have only a mild phenotype prior to activation of the cassette.

### B. Production of Founders with PKD1^{FLEX-CreER2} by Cloning

Male and female cells from A, above, are cloned by chromatin transfer and transferred to synchronized recipients. Multiple female recipient animals are used as needed to produce cloned ADPKD founders of each sex with conditional alleles. In some embodiments, a pregnancy rate of 50% with an average of 2-3 piglets is expected. Hence, there is a greater than an 80% chance of producing 3+ pregnancies for each sex, producing at least 6 piglets of each sex for this study. This number is sufficient to perform preliminary characterization of the phenotypes +/− Tamoxifen administration in part C., below. In some embodiments, further cloning is used to generate male founders with homozygous PKD1^{FLEX-CreER2} as these are an ideal paternal line for product propagation by intercross with the PKD1^{+/-} breeding herd. Genotypes are confirmed in all newborn founders by PCR and Sanger sequencing.

### C. Tamoxifen Activation and Cyst Development

Baseline phenotype of newborn is evaluated by ultrasound and blood chemistry within 48 hours of birth, analogous to the methods performed in Example 1. Half of the founders for each sex are randomly assigned to two groups, one receiving tamoxifen treatment, and the other receiving mock injections. To ensure accurate and consistent dosing, freshly prepared tamoxifen is administered for 5 consecutive days by intraperitoneal injection at a dosage of 75 mg/kg body weight. Blood is drawn again after day 5 and again 2 weeks later to evaluate EGFP expression (or loss there-of) by FACS, and to detect recombination by PCR. Blood chemistry and ultrasound analysis will continue at day 30 and monthly thereafter to study completion at 7 months of age. At this time, or earlier if necessitated by ESRD, animals are euthanized, and tissues collected for histopathological analysis and protein analysis as indicated in Example 3. In addition, recombination is quantified in kidney and liver specimens by enumerating EGFP positive vs negative cells and on the molecular level by qPCR.

### D. Results and Alternative Embodiments

Several positive clones were pooled from a successfully cloned donor line to protect from the variation in the success of somatic cell transfer from individual clones or donor lines. Based on preliminary data, it is expected that the compound heterozygotes will not have a cystic phenotype at birth and very mild disease. However, it is possible that the PKD1^{FLEX-CreER2} allele will express at slightly lower levels than the wild type allele due to interference with normal splicing. Though this may be possible, the optimized design in Example 7 should limit this pitfall. Even if the levels of PC1 are reduced by 25%, it is expected that the initial phenotype is milder than the RC-RC or RC-null animals, prior to activation of the conditional cassette. Additionally, as noted above, preliminary data showed one PKD+/− animal having a robust newborn phenotype, presumably due to aberrant reprogramming in the cloning process. Any newborn founders displaying this phenotype are not used in the tamoxifen induction experiment.

Whereas the conditional cassette is a novel all-in-one locus that results in production of a mutant PC1 protein upon activation, the outcome should, to some extent, approach those of conditional mouse models. As with pigs taught herein, mouse models utilize activation by Cre^{ER2} either by oral or intraperitoneal administration of tamoxifen. In all cases, despite PC1 inactivation in as little as 10-20 percent of the kidney, disease progression is robust and consistent since cystgenesis is a clonal event. Hence, the percent recombination is of little concern as long as it is at a consistent level in the experimental pigs. Finally, mouse conditional models revealed an important switch around the time when murine renal development is complete (˜P13). If PC1 is inactivated before ˜P13, very rapidly progressive cystic disease develops, while thereafter activation results in a very slowly progressive disease. By treating newborn piglets, it is intended to activate the disease far prior to the analogous P13 time point in mice, hence a strong, progressive disease is expected. Later induction time points in the pig may reduce the severity of disease, providing yet alternative time courses of disease progression tailored to the demands of preclinical protocols.

Based on the established breeding herd, the in vivo portion of Examples 4 and 5 should be completed within the 18 months of the beginning of the experiments outlined in the Examples. Post-collection image analysis, histopathology, and summation of results requires approximately 6 months. Those of skill in the art will appreciate that the timelines for Examples 6 and 7 enables multiple iteration testing of the inducible cassette in Example 6. In some embodiments, 7 months is a time frame available to observe whether tamoxifen induced piglets develop kidney cysts. These innovative, benchmarked models are of exceptional value to drug and devices companies, enabling development novel therapeutics and imaging strategies that can improve patient care.

### Example 8

### Evaluation of TALEN or RGEN Mediated Editing of Porcine PKHD1 to Model ARPKD.

Specific mutations in the PKHD1 gene that are diagnostic of PKD have been mapped to Exons 3, 58 and 61 (FIG. 5A, FIG. 5B, FIG. 5C, FIG. 5D, and FIG. 5E). Porcine genomes are edited to express homologous mutations as those in humans.

### A. Establishment of Effective TALEN or RGEN Reagents Targeted to Exon 3 of PKHD1: Introgression of T36M into the Porcine Gene

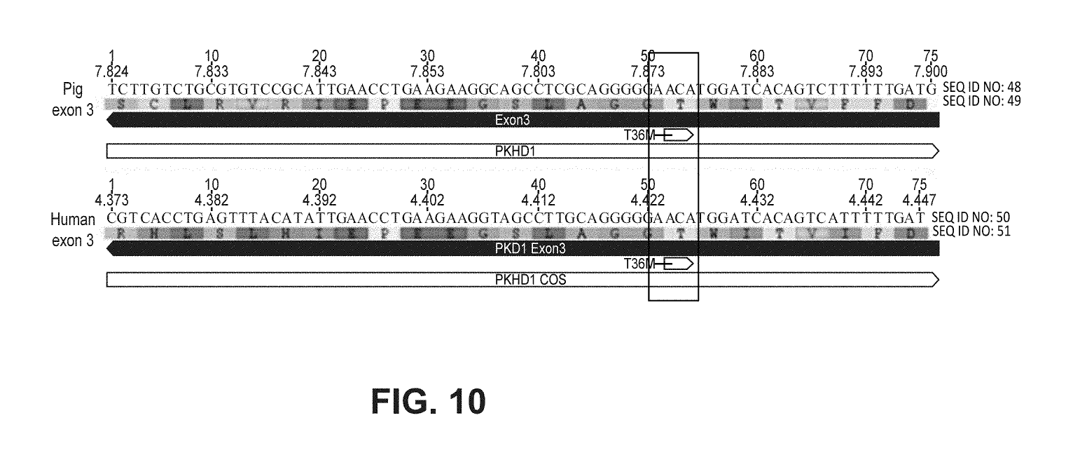

The T36M mutation of PKHD1 is the most prevalent mutation identified in ARPKD patients. Tyrosine 36 is conserved in exon 3 of the pig, along with the majority of other residues in exon 3 (FIG. 5D and FIG. 10) when compared to human; hence the orthologous T36M mutation is also predicted to be pathogenic in swine (FIG. 9D).

A number of quality sites for TALENs and CRISPRs are ideally positioned for HDR mediated introgression the T36M mutation (Table 7, Table 8, Table 9). The assembled reagents are tested in pig fetal fibroblasts and embryos using standard operating procedures (SOPs) (FIG. 9B). Using these methods, hundreds of loci in pig and cattle have been successfully targeted, with efficiencies ranging from 5-75% in cultured cells.

### B: Conditions for High Penetrance Editing of In Vitro Produced Porcine Embryos

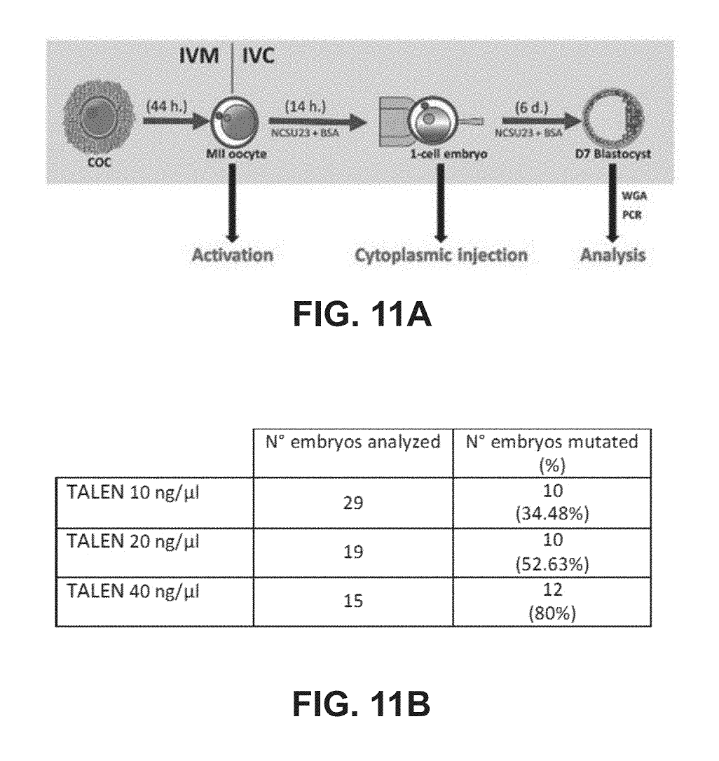

The best performing combinations of TALENs and CRISPRs identified above, are evaluated in vitro by injection of in vitro matured (IVM) parthenogenetic pig embryos (FIG. 9C). Parthenogenetic embryos have been found to be a reliable and low-cost alternative to in vitro fertilized embryos for these evaluations. In a small pilot study targeting porcine PDX1, 80% of alleles injected with a TALEN dosage of 40 ng/μl were able to be mutated, with nearly half of those directed to a particular edit by a homology template (FIG. 11B). FIG. 11A illustrates the steps required in production of in vitro matured pig embryos for microinjection. FIG. 11B illustrates the difference in percent success which using different concentrations of editing reagents for the PDX1 gene. A similar dosage escalation study is used to optimize for T36M introgression that balances survival of embryos in vitro with editing efficiency.

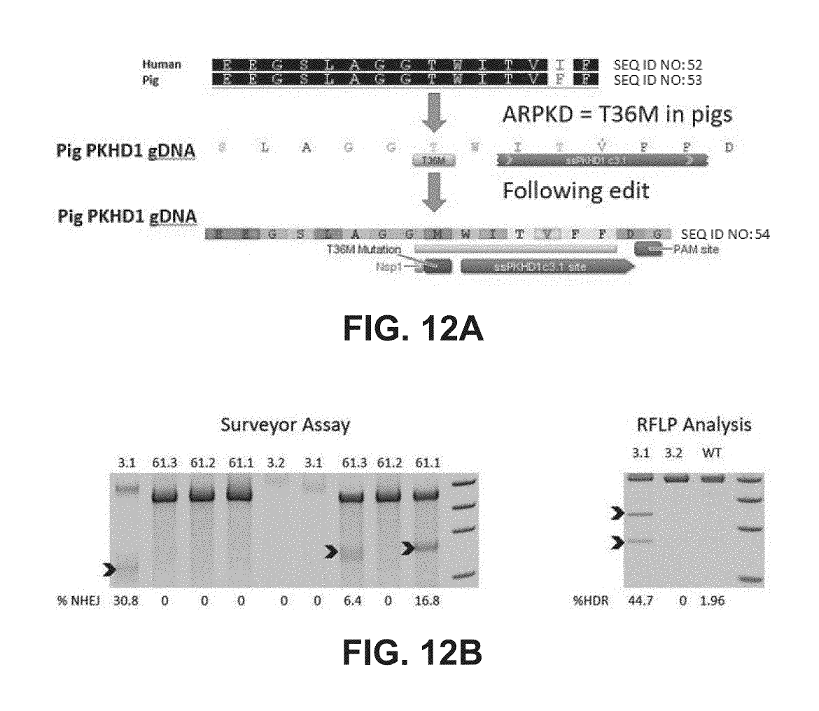

For introgression of T36M into the porcine genome, two CRISPR Cas9 gRNAs and TALENs were tested in pig fibroblasts and embryos to optimize conditions (FIG. 12A, FIG. 12B, Table 7, Table 8, Table 9). Of these, ssPKHD1 CRISPR 3.1 showed the greatest activity in cells and was subsequently tested with a homology dependent repair template (HDR) ssPKHD1 3.1 36 T to M (Table 10) that encodes the T36M mutation and a novel Nsp1 restriction site for RFLP genotyping. Co-transfection of CRISPR 3.1 and the HDR template stimulated HDR up to 44.7% (FIG. 12B) in cultured cells; hence this combination of CRISPR and template was chosen for further characterization in embryos.

TABLE 7
Summary of TALEN and CRISPR reagents in cell and embryos.

| | Reagent | In Fibroblast Cells | In Fibroblast Cells | In |
|---|---|---|---|---|
| Target | Name | Incubated at 30° | Incubated at 37° | Embryos |
| T36M | ssPKHD1 | 0% cutting | 30.8% cutting | 80% RFLP |
| | CRISPR 3.1 | RFLP detected, too | RFLP 44.7% | |
| | | faint to quantify | | |
| T36M | ssPKHD1 | 0% cutting at 30 | 0% cutting at 37 | 0% RFLP |
| | CRISPR 3.2 | without HDR | without HDR | |
| | | RFLP detected, too | RFLP detected, too | |
| | | faint to quantify | faint to quantify | |
| I3553T | ssPKHD1 | 16.8% cutting at 30 | 0% cutting at 37 | Not tested |
| | TALEN 61.1 | | | |
| I3553T | ssPKHD1 | 0% cutting at 30 | 0% cutting at 37 | Not tested |
| | TALEN 61.2 | | | |
| I3553T | ssPKHD1 | 6.4% cutting at 30 | 0% cutting at 37 | Not tested |
| | TALEN 61.3 | | | |
| R2990Q | ssPKHD1 | 0% cutting | 0% cutting | Not yet |
| | TALEN 65.1 | | | determined |
| R2990Q | ssPKHD1 | Cutting detected, too | Cutting detected, too | Not yet |
| | TALEN 65.2 | faint to quantify | faint to quantify | determined |
| | | 25% RFLP | 0% RFLP | |
| R2990Q | ssPKHD1 | Cutting detected, too | Cutting detected, too | 89% |
| | CRISPR 65.1 | faint to quantify | faint to quantify | |
| | | 7.3% RFLP | 11% RFLP | |

TABLE 8
TALEN RVD SEQUENCES

| | | Nucleotide Binding Sequence |
|---|---|---|
| Name | RVD Sequence | (5′-3′)/SEQ ID NO: |
| ssPKHD1 | HD NI NG NN NN NI NG NI NI HD HD | CATGGATAACCTCTTAT |
| 61.1 L | NGHD NG NG NI NG | SEQ ID NO: 84 |
| ssPKHD1 | NG HD NI NI HD NI NN NN HD NG HD | TCAACAGGCTCCTCTCC |
| 61.1R | HD NG HD NG HD HD | SEQ ID NO: 86 |
| ssPKHD1 | NI NI HD HD NG HD NG NG NI NG NI | AACCTCTTATATGTTGT |
| 61.2L | NG NN NG NG NN NG | SEQ ID NO: 88 |
| ssPKHD1 | NN NI NN HD NN NG NI NG NG NG HD | GAGCGTATTTCAACAG |
| 61.2R | NI NI HD NI NN | SEQ ID NO: 90 |
| ssPKHD1 | HD NG NG NI NG NI NG NN NG NG NN | CTTATATGTTATCCT |
| 61.3L | NG HD HD NG | SEQ ID NO: 92 |
| ssPKHD1 | NN NI NN HD NN NG NI NG NG NG HD | GAGCGTATTTCAACAG |
| 61.3 R | NI NI HD NI NN | SEQ ID NO: 94 |
| ssPKHD1 | NN NN NI HD NI NG HD NI NN HDNG | GGACATCAGCTGATCGAG |
| 65.1 L | NN NI NG HD NN NI NN | SEQ ID NO: 96 |
| ssPKHD1 | NI NG NI HD HD NI NI HD NG HD NN NI | ATACCAACTCGACCCCC |
| 65. R | HD HD HD HD HD | SEQ ID NO: 98 |
| ssPKHD1 | HD NI NN HD NG NN NI NG HD NN NI | CAGCTGATCGAGCTCCT |
| 65.2 L | NN HD NG HD HD NG | SEQ ID NO: 100 |
| ssPKHD1 | NI NG NI HD HD NI NI HD NG HD NN NI | ATACCAACTCGACCC |
| 65.2 R | HD HD HD | SEQ ID NO: 102 |

TABLE 9
CRISPR guide RNA sequence.

| Name/ | Nucleotide Binding Sequence |
|---|---|
| SEQ ID NO: | (5′-3′) PAM |
| ssPKHD1 CRISPR 3.1 | GGATTACCGTGTTCTTCGA -TGG |
| SEQ ID NO: 103 | |
| ssPKHD1 CRISPR 3.2 | GCAGCCTCGCAGGGGGAACA-TGG |
| SEQ ID NO: 104 | |
| ssPKHD1 CRISPR 65.1 | GGGGTCGAGTTGGTATATTG-TGG |
| SEQ ID NO: 105 | |

TABLE 10
Repair Templates

| Template/ | |
|---|---|
| SEQ ID NO: | HDR TEMPLATE (5′-3′) |
| ssPKHD1 3.1 36 T | gcgtgtccgcattgaacctgaagaaggcagcctcgcagggggCaTGtggatTacCgtGttCtt |
| to M | cgatggtaggtgcggtcttcctacagc CAPITAL LETTERS = SNPS, |
| SEQ ID NO: 106 | ITALIC = T36M, UNDERLINE = NSP1 SITE |
| ssPKHD1 65.1 3240 | aactggacatcagctgatcgagctccttccagtcccagaggCggCcAagtGggAatTCtgtgg |
| R to Q | cctgtgttcacctcagaaccaaatcgg |
| SEQ ID NO: 107 | CAPITAL LETTERS = SNPS, ITALIC = R3240Q, UNDERLINE = |
| | ECORI SITE |

TABLE 11
Primers

| Name | Sequence (5′-3′) | SEQ ID NO: |
|---|---|---|
| ssPKHD1 E3 NJ | TCTTGGACCACGGACCTACT | SEQ ID NO: |
| F1 | | 108 |
| ssPKHD1 E3 NJ | TTGGGGTTCCTGGAGTACCA | SEQ ID NO: |
| R1 | | 109 |
| ssPKHD1 E65 NJ | GGTAGACAATGCTGTTGGC | SEQ ID NO: |
| F2 | | 29 |
| ssPKHD1 E65 R2 | TGCTCAACTAGACAGCAGGC- | SEQ ID NO: |
| | | 30 |

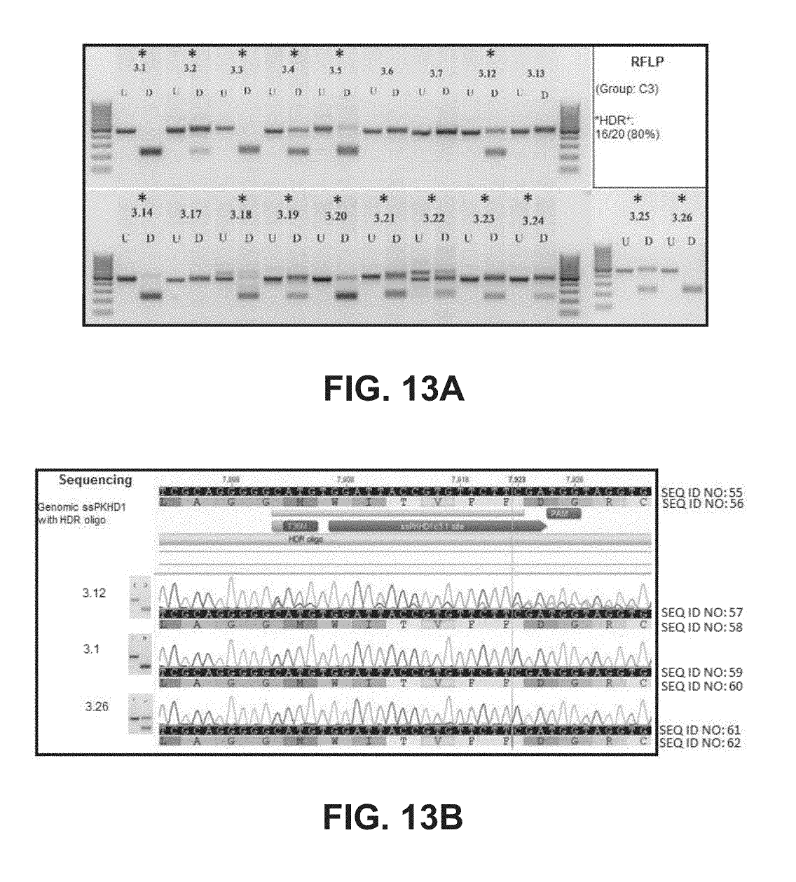

Initially, the toxicity of ssPKHD1 CRISPR 3.1 with the HDR oligo (single strand oligo (ssODN) ssPKHD1 3.1 36 T to M was assessed by embryo development analysis after cytoplasmic injection of three concentrations (C) in ng/μl of Cas9 mRNA/gRNA/ssODN: C1 (100/50/50), C2 (50/25/50) and C3 (25/12.5/50). The blastocyst formation rate was high in each group, but highest in the C3 group where the concentration of CRISPR components are the lowest. Next, the frequency of HDR and NHEJ activities were determined based on single embryo analysis in the three evaluated concentrations. Up to 95% of embryos injected with a CRISPR/Cas9 at a dosage of 25-100 ng/μl were mutated (Table 12). HDR efficiency was inversely associated with Cas9 dosage (50 vs 80% HDR) from highest to lowest concentration (Table 12 and FIG. 13A) likely a result of hyperactive cutting in the high concentration group. The fidelity of HDR events was further confirmed by sequencing (FIG. 13B) which confirms that the wild type triplet “ACA” coding, for threonine has been converted to “ATG” coding for methionine (FIG. 9A) using the template HDR sequence. Overall, condition C3, 25 ng/ul of Cas9, 12.5 ng/ul of gRNA and 50 ng/μl of ssHDR, was optimal for both blastocyst formation rate and HDR activity. If this site proves problematic, alternative missense mutations commonly observed in more than one family of ARPKD patients, such as I3553T, I222V, P805L or R3229Q are introgressed.

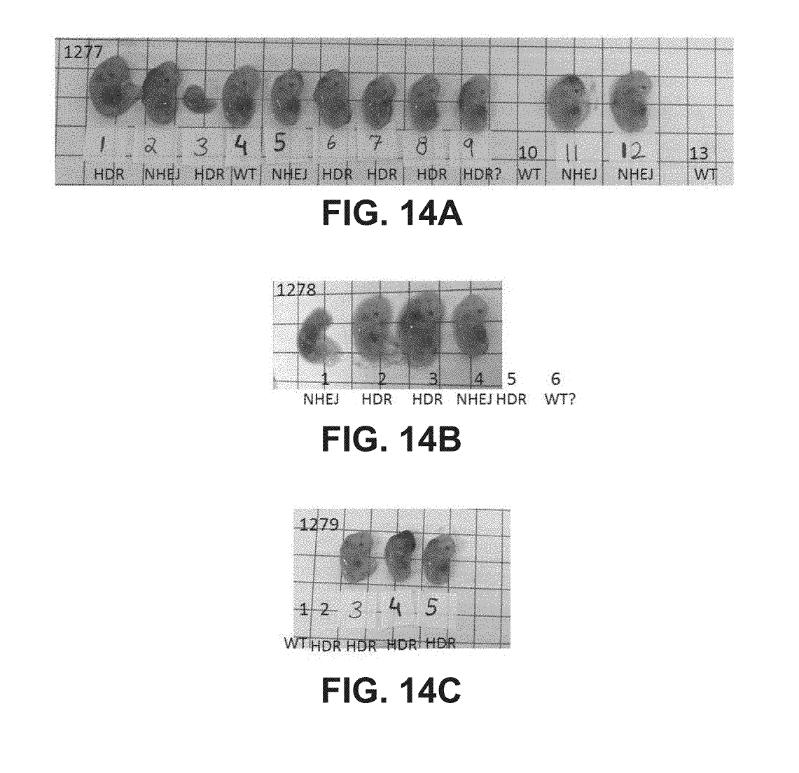

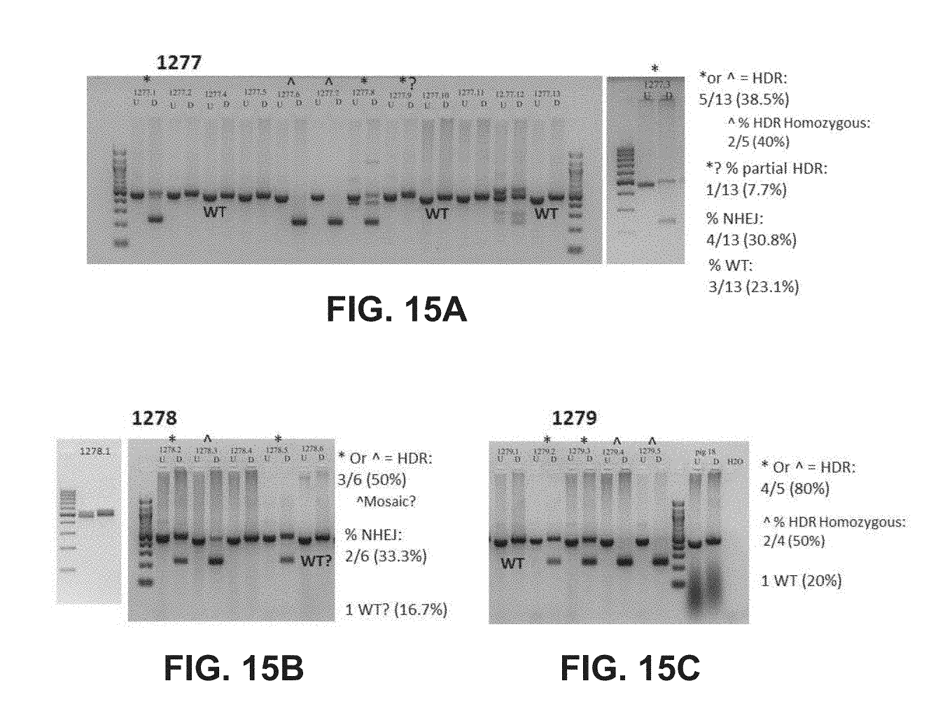

A subsequent round of T36M injections into embryos was conducted according to condition C3, above. Instead of in vitro culture to the blastocysts stage, these embryos were transferred to three recipient sows. A total of 81, 84 and 83 day-1 injected embryos were surgically transferred to recipient 1277, 1278 and 1279, respectively. At 29 days of gestation, the uterus was removed from recipients and fetuses collected (FIG. 14A, FIG. 14B and FIG. 14C respectively). Fetuses 10, 13 (FIG. 14A) 5, 6 (FIG. 14B) and 1 and 2 (FIG. 14C) were resorbed and not visible. The embryos were genotyped using RFLP assay (FIG. 15A, FIG. 15B, and FIG. 15C) and DNA sequencing. Any with an HDR allele (T36M) are indicated. The total number exhibiting HDR was 38.5% (FIG. 15A), 50% (FIG. 15B), and 80% (FIG. 15C) by RFLP assay. While overall mutation rate of 79% and HDR frequency (50%) was lower than in vitro test (Table 12) for condition C3, it still demonstrates the in vitro tests are predictive of success in establishment of active pregnancies with fetuses carrying the intended edits.

TABLE 12
Efficient T36M introgression in embryos.

| Group ng/μl | | | | |
|---|---|---|---|---|
| (Cas9/gRNA/ | Analyzed | HDR⁺ | NHEJ⁻ | Mutated |
| ssODN) | n | n (%) | n (%) | n (%) |
| C1 | 18 | 19 (50.0%) | 7 (38.8%) | 17 (94.4%) |
| (100/50/50) | | | | |
| C2 | 20 | 11 (55.0%) | 8 (40.0%) | 19 (95.0%) |
| (50/25/50) | | | | |
| C3 | 20 | 16 (80.0%) | 3 (15.0%) | 19 (95.0%) |
| (25/12.5/50) | | | | |

The frequency of HDR and NHEJ activities were determined based on single embryo analysis in the 3 conditions evaluated.

### Example 9

Evaluation of TALEN or RGEN Mediated Editing Engineering of R3229Q into Porcine Embryos

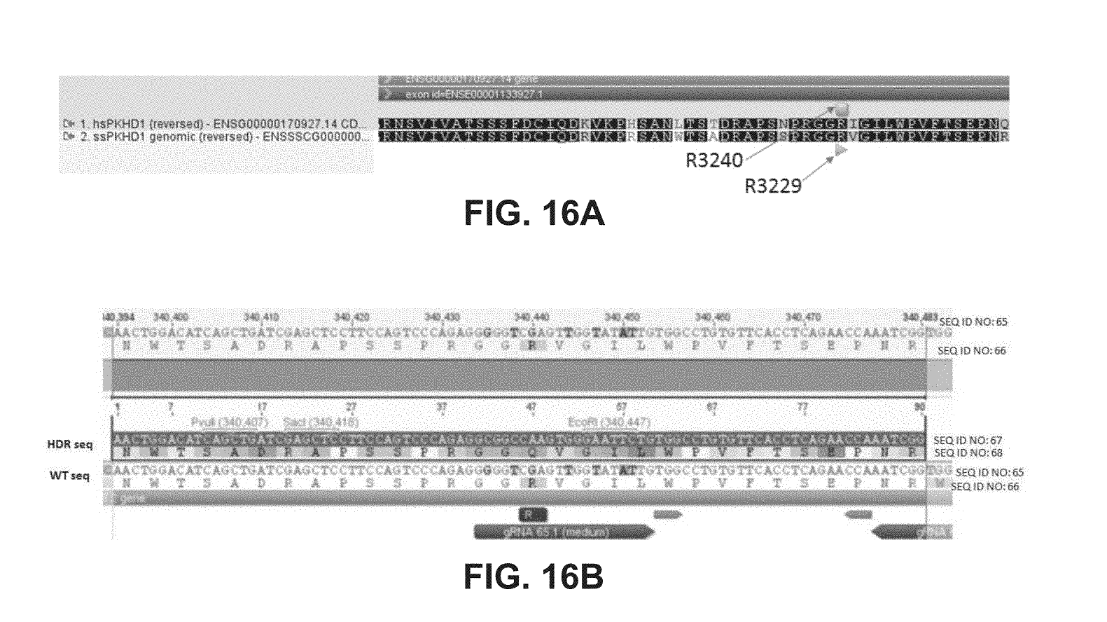

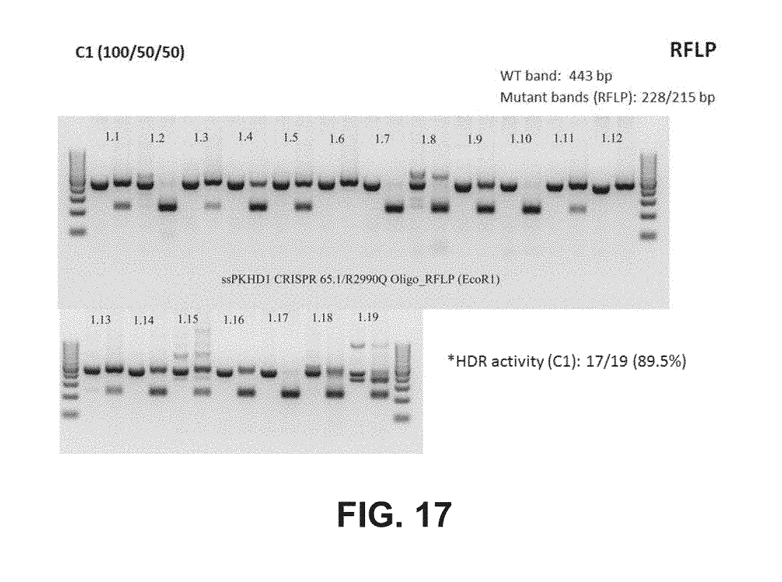

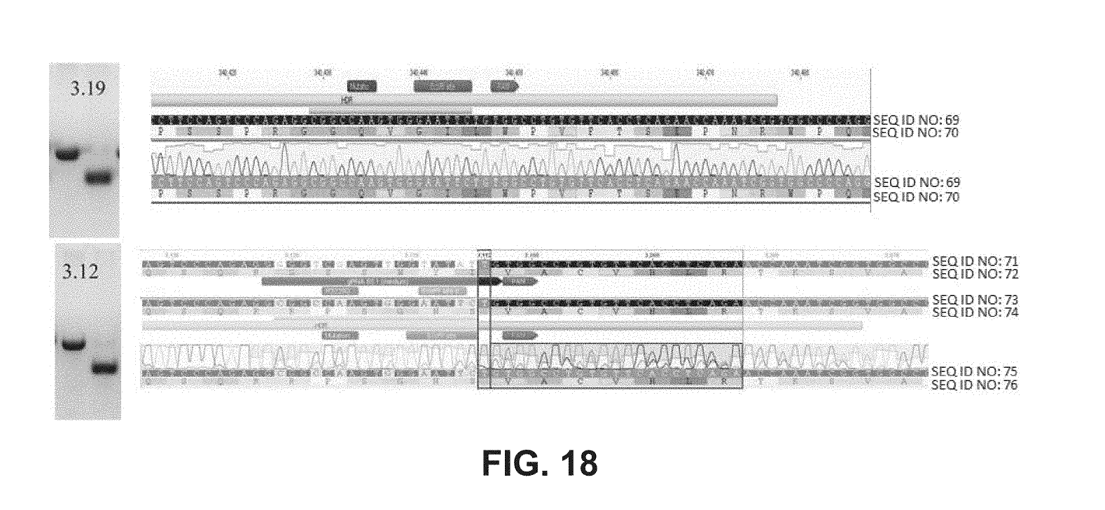

By the same process as Example 8, R3229Q was engineered into the PKHD1 gene. R3229Q is the equivalent mutation to R3240Q in humans (FIG. 16A), which in a homozygous state causes moderate ARPKD. Also, as noted previously, the annotation of PKHD1 has changed since initial contigs placed R3240Q in exon 58 versus exon 65. Also, R3240Q in humans, maps to different residue numbers in pigs based on transcript selected. FIG. 5B clearly shows the porcine residue targeted relative to the human sequence. Hence, R3240Q or R3229Q are used interchangeably. A number of TALENs and CRISPR/Cas9 guide RNA's were developed for this target, and of those ssPKHD1 CRISPR 65.1 had the greatest activity (Table 7-9, FIG. 16B). Injections into parthenogenetic embryos were performed as before, with two conditions using a ssODN as the homology template ssPKHD1 65.1 3240 R to Q (Table 10); C1 (100/50/50) (Cas9 mRNA/gRNA65.1/homology template, all ng/μl) and C3 (25/12.5/50) (Cas9 mRNA/gRNA65.1/homology template, all ng/μl) (Table 9). Blastocysts analyzed under the C1 condition demonstrate highly efficient introgression of the R3229Q allele (˜90%) with several homozygous examples, 1.2, 1.7, 1.10 and 1.17 (FIG. 17). HDR was also prevalent in the C3 condition (75%) whereas total mutation rate (indels+HDR alleles) was 100 and 95% for C1 and C3 respectively. FIG. 18 shows sequence confirmation of two blastocysts from the C3 condition. Blastocyst 3.19 is homozygous for R3229Q while 3.12 has one HDR allele and one indel allele.

TABLE 13
R3229Q introgression in porcine parthenogenetic embryos.

| Group | Analyzed n | HDR n (%) | NHEJ n (%) | Mutated n (%) |
|---|---|---|---|---|
| C1 | 19 | 17 (89.50%) | 2 (10.5%) | 19 (100%) |
| C3 | 20 | 15 (75.0%) | 4 (15.0%) | 19 (95.0%) |

### Example 10

Characterization of Kidney and Liver Phenotype in Piglets Born after Microinjection.

Zygotes injected with conditions identified above are implanted into 6 synchronized recipient gilts and carried to parturition. In some embodiments, it is expected that 3-4 pregnancies will reach full term. During the pregnancy, fetal development is monitored by ultrasonography to check for signs of oligohydramnios. If ultrasonography reveals signs of oligohydramnios, piglets are delivered by C-section for evaluation and collection of amniotic fluid samples. Furthermore, cord blood is collected to evaluate newborn kidney and liver function by running liver and kidney panels (Idex Bioresearch). Newborn pigs are monitored closely in the first day of life for visible and biochemical markers of ARPKD, and those with severe disease are euthanized for necropsy. Kidney, liver, bile ducts, pancreas and lungs are closely examined grossly prior to histological and molecular characterization. Tail clips are collected from all animals for genotypic analysis.

Animals surviving past day 1 are expected to be either heterozygous for PKHD1 mutations, compound heterozygotes for a LOF mutation and a missense allele (such as T36M), or homozygous for missense alleles (FIG. 9D). These animals are monitored starting at day 2 on a monthly basis by ultrasonography using the CTS-8800V-Plus apparatus once per month from 1-6 months. This analysis is used to quantify the total number of cysts found in each kidney per animal for each genotype. Cysts will need to at least 5 mm in diameter to be recorded as cysts. In addition, the volume of each kidney is determined and the total kidney volume (TKV), including both kidneys, are recorded. The TKV are determined using length, width and depth measures and employing the ellipsoid formula.

Monitoring of renal function by blood and urine analysis: A 2 ml arterial blood sample are collected monthly at the time of the imaging in EDTA tubes. A urine collection of 50 ml are obtained at the same time using a supra-pubic catheter. Measurements of blood and urine chemistries are performed on a contract basis with Idex Bioresearch. Serum creatinine levels in excess of 8 mg/dl would be an indication of severe uremia and that the pig would need to be euthanized for necropsy. Likewise, grossly elevate AST or ALT levels accompanied by jaundice would indicate severe liver disease and also be grounds for euthanasia.

Histological analysis of kidney and liver: At 6 months, pigs, except those chosen for breeding, are euthanized and representative kidney and liver tissue taken for histological analysis. Renal tissue is cut from the excised kidney in the area of the hilum including both cortex and medulla. Both the renal and liver tissue are fixed in 10% neutral buffered formalin, dehydrated, and embedded in paraffin, per standard techniques. As necessary, renal tissue, adjacent to the formalin tissue, will also be flash frozen and tissue embedded in OCT medium for frozen sections. Representative formalin fixed sections are stained with H&E and Masson's trichrome. Additional stains may also be employed, such as PAS and Sirius red and immunohistochemistry for the PKHD1 protein.

### Example 11

Characterization of Kidney and Liver Phenotype in Piglets Born after with T36M Reagents.

Founder animals for ARPKD were produced using the process of Example 8 with minor changes. The T36M reagents (ssPKHD1 CRISPR 3.1 and template ssPKHD1 3.1 36 T to M) were injected into in vivo produced porcine embryos at the 1 or 2 cell stage. Condition C3 was used for injection and the 60 resulting embryos were split equally between to synchronized recipients. A total of 22 piglets were born, 14 from one surrogate (litter ID 115) and 8 from the other (litter ID 116). Newborn piglets were visually assessed for fitness and four, 115-1, 115-2, 115-3 and 115-8 were reluctant to suckle from their mothers and had visible abdominal distension (Table 14). These piglets were euthanized and revealed severely cystic and enlarged kidneys as well as severely enlarged livers with abnormal gallbladders (FIG. 18). Each of the remaining piglets was analyzed for a visible phenotype at 20 days of age (Table 14). Genotype of the piglet was a strong indicator of disease severity as 115-1, 115-2, 115-3 and 115-8 are predicted to have null mutations on each allele while mild cases had some combination of T36M mutation or single allele mutations (Table 14). Overall, ˜55% of offspring were mutant with 5 showing T36M mutations (Table 15). This rate is lower than observed above. A key difference is the embryos source. Parthenogenetic embryos were used in previous experiments where timing of activation is tightly controlled. In contrast, this experiment used in vivo produced embryos (bred sows, embryos collected ˜18 hours post fertilization) where timing is variable. However, the hypothesis that a variety of disease alleles and severities could be produced in a single litter was confirmed.

TABLE 14

| ID | Phenotype | Allele 1 | Allele 2 |
|---|---|---|---|
| 115-1 | Severe | 22 bp deletion with | 22 bp deletion with |
| | | down-stream stop codon | down-stream stop codon |
| 115-2 | Severe | 4 bp deletion | 4 bp deletion |
| 115-3 | Severe | Insertion of a T | Insertion of a T |
| | | leading a stop codon | leading a stop codon |
| 115-8 | Severe | 26 bp insertion | 26 bp insertion with |
| | | | T36M mutation |
| 116-1 | Mild | T36M mutation | 4 bp deletion |
| 115-4 | Mild | T36M mutation | T36M mutation |
| 116-4 | No disease | T36M mutation | T36M mutation |
| | observed | | |
| 116-7 | No disease | T36M mutation | T36M mutation |
| | observed | | |
| 116-8 | Moderate | 10 bp deletion | WT |
| 115-14 | No disease | T36M mutation | WT |
| | observed | | |
| 116-5 | No disease | T36M mutation | WT |
| | observed | | |
| 115-5 | No disease | WT | WT |
| | observed | | |
| 115-6 | No disease | WT | WT |
| | observed | | |
| 115-7 | No disease | WT | WT |
| | observed | | |
| 115-9 | No disease | WT | WT |
| | observed | | |
| 115-10 | No disease | WT | WT |
| | observed | | |
| 115-11 | No disease | WT | WT |
| | observed | | |
| 115-12 | No disease | WT | WT |
| | observed | | |
| 115-13 | No disease | WT | WT |
| | observed | | |
| 116-2 | No disease | WT | WT |
| | observed | | |
| 116-3 | No disease | WT | WT |
| | observed | | |
| 116-6 | No disease | WT | WT |
| | observed | | |

Litter 115 and 116 piglets after embryo microinjection with ssPKDH1 CRISPR 3.1 and T36M HDR template. Genotype is reported for each allele and disease state was evaluated by low resolution ultrasound in remaining animals. Severe animals were euthanized within 48 hours of birth, the remaining phenotype evaluations are from 20-day old piglets.

TABLE 15

| | | Heterozygous | Homozygous |
|---|---|---|---|
| Wild type | Mutant | T36M | T36M |
| % (number) | % (number) | % (number) | % (number) |
| 45.5% (10/22) | 54.5% (12/22) | 9% (2/22) | 13.6% (3/22) |

### Example 12

Interbreeding of Heterozygous Carriers to Assess Phenotype: Genotype Consistency in the F1 Generation.

Preliminary data in porcine zygotes suggests that a high number of founders have biallelic mutation, though some heterozygous animals may be present. Breeding pairs are established and interbred to evaluate the disease in F1 piglets by the methods of EXAMPLE 9.

The proposed gene-editing method delivers a testable hypothesis enabling solid preliminary data regarding precise alterations in an animal that results in ARPKD. Additionally, this research focus could provide valuable progress for all four identified types of autosomal recessive PKD (perinatal, neonatal, infantile, and juvenile) with a specific aim of addressing the most challenging perinatal and neonatal presentations. The development of specific edited pigs as described here can greatly enhance ARPDH research and the development of treatment options for preclinical testing.

### Example 13

Generation of ADPKD Models and Imaging of ARPKD Models.

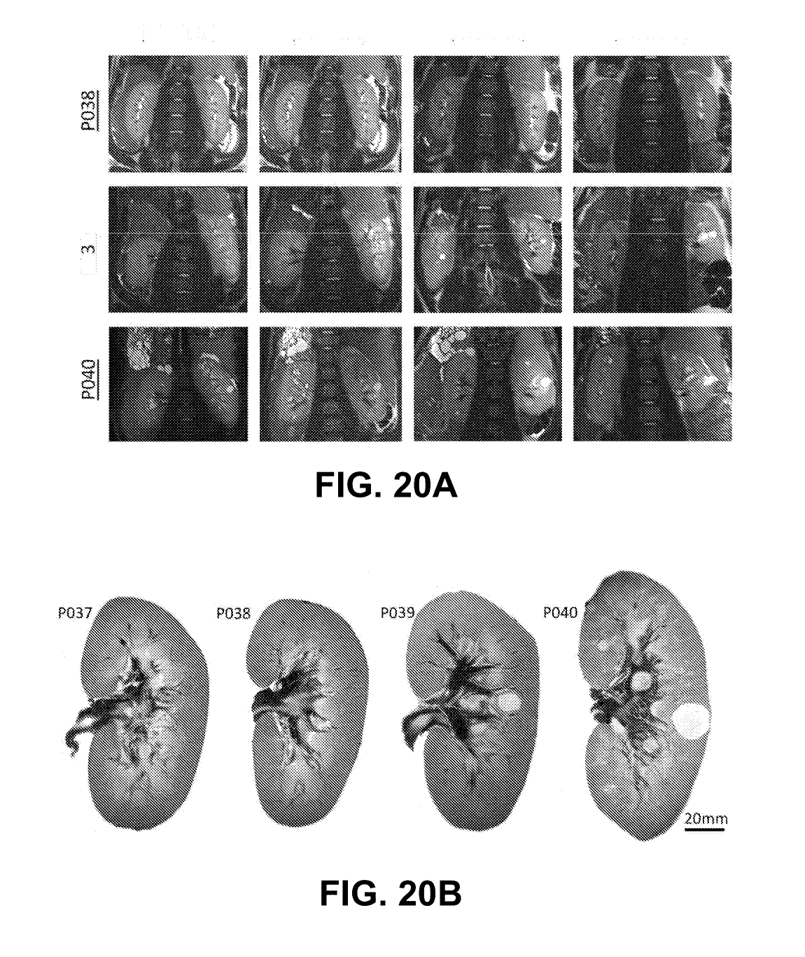

Six litters of pigs were produced by intercross of PKD1 RC and PKD1 null heterozygotes (Table 2). The generated genotypes and pigs modified at the PKHD1 gene as a model of autosomal recessive polycystic kidney disease (ARPKD) were characterized. Imaging results from the ARPKD animals are presented in FIG. 20A and FIG. 20B. No cysts were observed in newborn ADPKD pigs. A summary of pigs examined is presented in Table 16.

TABLE 16
Study groups and enrollment summary.

| | | | | Completed 6- | |
|---|---|---|---|---|---|
| | | Group | | month trial | |
| Group | | Size | | (serum and | |
| number | Genotype | Proposed | Enrolled | urine analysis) | Imaged |
| 1 | RC/RC | N = 7 | 0 | 0 | |
| 2 | WT | N = 7 | 3 | 3 | 0 |
| 3 | RC/WT | N = 7 | 1 | 0 | Not |
| | | | | | Planned |
| 4 | Null/WT | N = 7 | 5 | 5 | Not |
| | | | | | Planned |
| 5 | ARPKD* | N = 3 | 3 | 3 | 4 |
| | Totals | 31 | 12 | 12 | 4 |

The initial design was to enroll a total of 21 animals of either sex, n=7 each for RC-RC and wild type pigs starting at 30 days of age and monitored to 6 months of age by monthly imaging and serum analysis (Table 16). RC/WT or Null/WT animals were also considered to be an informative model for early stages of the disease. For this reason, we added groups 3 and 4 (Table 16), to monitor biochemical factors and for evaluation of kidney disease by necropsy at 6 months of age. In addition, ARPKD animals were enrolled as a positive control group with known kidney and liver disease for comparison (Group 5). No elevated values in kidney and liver panels were observed in groups 2, 3, 4, as expected. Lastly, we used the ARPKD group of animals as our first enrollees into the imaging protocol to establish and troubleshoot the process of imaging and analysis of both diseased and normal pigs. Magnetic resonance images of the four ARPKD models are presented over a time course of 4 months in FIG. 20A and FIG. 20B.

As shown in FIG. 20A, ARPKD models were imaged at 3-6 months of age. Mild cystic kidney phenotype in P039 and P040 and pronounced cystic liver in P040 were observed. FIG. 20B shows ex-vivo MR imaging of kidneys from the same animals. Genotypes: P037-PKHD1 T36M/T36M; P038-PKHD1 T36M/+; P039-PKHD1 T36M/T36M; P040-PKHD1 Mosaic of Wt and frame-shift alleles. The ARPKD models were produced by embryo microinjection.

### EMBODIMENTS

The following paragraphs enumerated consecutively from 1 through 35 provide for various additional aspects of the present disclosure. In one embodiment, in a first embodiment, 1 is provided:

1. A genetically edited pig as a model for studying polycystic kidney disease (PKD), wherein the genome of the edited pig comprises at least one edited gene or combination of edited genes selected from:

(a): (i) human PKD1 and/or PKD2 gene, and/or (ii) porcine PKD1 and/or PKD2 gene; or
(b): (i) human PKHD1 and/or (ii) porcine PKHD1;
(c): combinations of (a) and (b); wherein the edited pig expresses at least one phenotype associated with polycystic kidney disease.

2. The edited pig according to embodiment 1, wherein the pig is a mini-pig.

3. The edited pig according to any of embodiments 1-2, wherein the mini-pig is selected from Ossabaw, Yucatan, Bama Xiang Zhu and Goettingen.

4. The edited pig according to any of embodiments 1-3, wherein the genome of the edited pig comprises at least one edited porcine PKD1 and/or PKD2 gene or one edited porcine PKDH1 gene comprising at least one mutation.

5. The edited pig according to any of embodiments 1-4, wherein at least one phenotype associated with PKD is cyst formation in kidney, liver, and seminal vesicles, high blood pressure, headaches, abdominal pain, blood in the urine, excessive urination and back pain.

6. The edited pig according to any of embodiments 1-5, wherein the phenotype occurs after 6 months.

7. The edited pig according to any of embodiments 1-6, wherein the phenotype is congenital.

8. The edited pig according to any of embodiments 1-7, wherein the phenotype is inducible.

9. The edited pig according to any of embodiments 1-8, wherein the phenotype is induced by administration of tamoxifen or tetracycline or their agonists.

10. The edited pig of any of embodiments 1-9, wherein the pig is heterozygous for the edit.

11. The edited pig of any of embodiments 1-10, wherein the pig is homozygous for the edit.

12. The edited pig of any of embodiments 1-11, wherein the pig is compound heterozygous for the edit.

13. A genetically edited porcine blastocyst derived from the genetically edited pig of any of embodiments 1-12, wherein the genome of the edited porcine blastocyst comprises at least one edited gene or combination of edited genes selected from (i) human PKD1 and/or PKD2 gene, and (ii) porcine PKD1 and/or PKD2 gene or (iii) human PKHD1 and/or (iv) porcine PKHD1.

14. A genetically edited porcine embryo derived from the genetically edited pig as defined in any of embodiments 1-12, wherein the genome of the edited porcine embryo comprises at least one edited gene or combination of edited genes selected from:

(a): (i) human PKD1 and/or PKD2 gene, and/or (ii) porcine PKD1 and/or PKD2 gene; or
(b): (i) human PKHD1 and/or (ii) porcine PKHD1; wherein the edited embryo expresses at least one phenotype associated with polycystic kidney disease.

15. A genetically edited porcine fetus derived from the genetically edited pig as defined in any of embodiments 1-12, wherein the genome of the edited porcine fetus comprises at least one edited gene or combination of edited genes selected from (i) human PKD1 and/or PKD2 gene, and (ii) porcine PKD1 and/or PKD2 gene or (iii) human PKHD1 and/or (iv) porcine PKHD1.

16. A genetically edited porcine donor cell or cell nucleus derived from the genetically edited pig as defined in any of embodiments 1-12, wherein the genome of the edited porcine donor cell or cell nucleus comprises at least one edited gene or combination of edited genes selected from (i) human PKD1 and/or PKD2 gene, and (ii) porcine PKD1 and/or PKD2 gene or (iii) human PKHD1 and/or (iv) porcine PKHD1.

17. A method of making a swine model of PKD comprising: genetically modifying a native swine (i) PKD1 and/or (ii) PKD2 gene or (iii) PKHD1 to mimic mutations present in humans with PKD.

18. The method of embodiment 17, wherein the edit is made using Homology-Independent targeted integration (HITI) or Precise Integration into Target Chromosome (PITCh) technology.

19. The method of any of embodiments 17-18, wherein the edit comprises PKD1:R3277C and/or PKD1:R2220W and/or PKD1:R2213W, and/or PKD1 E5 KO, and/or PKHD1:T36M and/or PKHD1:R3240Q, a stop codon or a missence mutation.

20. The method of any of embodiments 17-19, wherein the PKD is ADPKD or ARPKD.

21. The method of any of embodiments 17-20, wherein the method further comprises introduction of an inducible cassette.

22. The method of any of embodiments 17-21, wherein tamoxifen or tetracycline or their agonists induces the cassette.

23. The method of any of embodiments 17-22, wherein the animal is in utero or older when PKD is induced.

24. The method of any of embodiments 17-23, wherein the genetic edit is made using a nuclease.

25. The method of any of embodiments 17-24, wherein the nuclease is zinc-finger nuclease, transcription activator-like effector nucleases (TALEN), meganuclease or Clustered Regularly Interspaced Short Palindromic Repeats (CRISPR).

26. The method of any of embodiments 17-25, wherein the animal is made by cytoplasmic injection of zygotes with gene-editing reagents.

27. The method of any of embodiments 17-26, wherein the animal is made by cytoplasmic injection, nuclear injection or cloning of primary cells with gene-editing reagents.

28. The method of any of embodiments 17-27, wherein the gene-editing reagents comprise a nuclease, an HDR template, and optionally guide RNA or a conditional allele cassette.

29. A method of making a genetically edited animal for PKD comprising using an all-in-one conditional allele cassette to model ADPKD or ARPDK and inducing the expression of a mutant PKD1 and/or PKD 2 allele or PKHD1 allele respectively.

30. The method of embodiment 29, wherein the conditional allele cassette comprises:

(i) a wild-type exon preceding a mutant form of the exon facing the opposite direction;
(ii) a CreER² recombinase; and
(iii) introduction of the cassette into an endogenous PKD1 and/or PKD2 or PKHD1 gene.

31. A method for evaluating the effect of a therapeutic treatment of PKD, comprising:

(i) providing the edited pig according to embodiment 1;
(ii) treating said pig with a pharmaceutical composition; and
(iii) evaluating the edited pig for an effect of the composition on a PKD disease phenotype expressed by the pig.

32. A method for screening the efficacy of a pharmaceutical composition for PKD comprising:

(i) providing the edited pig according to embodiment 1;
(ii) administering to said pig a pharmaceutical composition the efficacy of which is to be evaluated; and
(iii) evaluating the edited pig for an effect, if any, of the pharmaceutical composition on a PKD disease phenotype expressed by the edited pig.

33. A method for treatment of a human suffering from PKD comprising:

(i) providing the edited pig according to embodiment 1;
(ii) administering to said pig a pharmaceutical composition the efficacy of which is to be evaluated;
(iii) evaluating the effect, if any, of the pharmaceutical composition on a PKD disease phenotype expressed by the edited pig; and
(iv) treating a human being suffering from PKD based on the effects observed in the pig.

34. A method for screening the identification of biomarkers for PKD comprising:

(i) providing the edited pig according to embodiment 1;
(ii) identifying biomarkers correlating to PKD disease symptoms; and optionally
(iii) administering to said pig a pharmaceutical composition the efficacy of which is to be evaluated; and
(iv) evaluating the identified biomarkers of the edited pig for an effect, if any, of the pharmaceutical composition on a PKD disease phenotype expressed by the edited pig.

35. An all in one conditional expression cassette comprising:

(i) a wild-type exon preceding a mutant form of an exon facing the opposite direction;
(ii) a CreER² recombinase; and wherein the cassette is introduced into a native allele containing the exon and wherein the recombinase is inducible.

While this invention has been described in conjunction with the various exemplary embodiments outlined above, various alternatives, edits, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, edits, variations, improvements, and/or substantial equivalents of these exemplary embodiments.

1. A genetically edited pig or dog as a model for studying autosomal recessive polycystic kidney disease (ARPKD), wherein a genome of the genetically edited pig or dog comprises at least one genetic edit to a PKHD1 gene that is operably linked to an inducible system and the genetically edited pig or dog expresses at least one phenotype associated with ARPKD.

2. The genetically edited pig or dog of claim 1, wherein the at least one genetic edit to the PKHD1 gene comprises a biallelic mutation.

3. The genetically edited pig or dog of claim 1, wherein the at least one genetic edit to the PKHD1 gene comprises a premature stop codon, a truncation mutation, or a missense mutation.

4. The genetically edited pig or dog of claim 1, wherein the pig or dog is homozygous for the at least one genetic edit to the PKHD1 gene.

5. The genetically edited pig or dog of claim 1, wherein the pig or dog is heterozygous for the at least one genetic edit to the PKHD1 gene.

6. The genetically edited pig or dog of claim 1, wherein the at least one genetic edit to the PKHD1 gene comprises a T to M mutation at a position that corresponds to position 36 of a human PKHD1 amino acid sequence.

7. The genetically edited pig or dog of claim 1, wherein the at least one genetic edit to the PKHD1 gene comprises an R to Q mutation at a position that corresponds to position 3240 of a human PKHD1 amino acid sequence.

8. The genetically edited pig or dog of claim 1, wherein the at least one genetic edit to the PKHD1 gene results in a genotype of T36M/−, wherein 36 is a position that corresponds to position 36 of a human PKHD1 amino acid sequence and − is a knockout allele.

9. The genetically edited pig or dog of claim 1, wherein the at least one genetic edit to the PKHD1 gene results in a genotype of T36M/T36M, wherein 36 is a position that corresponds to position 36 of a human PKHD1 amino acid sequence.

10. The genetically edited pig or dog of claim 1, wherein the at least one genetic edit to the PKHD1 gene results in a genotype of −/−, wherein − is a knockout allele.

11. The genetically edited pig or dog of claim 1, wherein the at least one genetic edit to the PKHD1 gene results in a genotype of R3240Q/−, wherein 3240 is a position that corresponds to position 3240 of a human PKHD1 amino acid sequence and − is a knockout allele.

12. The genetically edited pig or dog of claim 1, wherein the at least one genetic edit to the PKHD1 gene results in a genotype of R3240Q/R3240Q, wherein 3240 is a position that corresponds to position 3240 of a human PKHD1 amino acid sequence.

13. (canceled)

14. The genetically edited pig or dog of claim 1, wherein the inducible system is a tetracycline-dependent regulatory system or a Cre/loxP recombinase system.

15. The genetically edited pig or dog of claim 1, wherein the inducible system comprises a conditional allele cassette.

16. The genetically edited pig or dog of claim 15, wherein the conditional allele cassette comprises a wild-type exon preceding a mutant form of the exon facing an opposite direction and a CreER² recombinase, wherein the conditional allele cassette is introduced into the PKHD1 gene.

17. The genetically edited pig or dog of claim 1, wherein the inducible system is induced by administering tamoxifen or tetracycline.

18. The genetically edited pig or dog of claim 1, wherein the inducible system is induced when the pig or dog is in utero, about 1 day old, 2 days old, 3 days old, 1 week old, 2 weeks old, 3 weeks old, or 6 weeks old.

19. The genetically edited pig or dog of claim 1, wherein the at least one phenotype associated with ARPKD is selected from the group consisting of cysts in kidney, cysts in liver, cysts in seminal vesicles, high blood pressure, headaches, abdominal pain, blood in urine, excessive urination, back pain, changes in blood urea nitrogen (BUN) levels, creatinine, BUN/creatinine, and BUN/cAMP.

20. The genetically edited pig or dog of claim 1, wherein the at least one phenotype is present when the genetically edited pig or dog is at least about 6 weeks and older.

21. The genetically edited pig or dog of claim 1, wherein the at least one phenotype is present at least about 6 weeks and older after induction of the inducible system.

22. The genetically edited pig or dog of claim 1, wherein the pig is a mini-pig.

23. The genetically edited pig or dog of claim 22, wherein the mini-pig is selected from the group consisting of Ossabaw, Yucatan, Bama Xiang Zhu and Goettingen.

24-114. (canceled)


SEQUENCE LISTING (119 sequences)

SEQ ID NO: 1 - skipped

SEQ ID NO: 2 - DNA, 15 bp, Sus scrofa
ccacaacgtcttccc

SEQ ID NO: 3 - skipped

SEQ ID NO: 4 - DNA, 17 bp, Sus scrofa
cggggccccagcagggc

SEQ ID NO: 5 - skipped

SEQ ID NO: 6 - DNA, 16 bp, Sus scrofa
gtgagccggccccagc

SEQ ID NO: 7 - skipped

SEQ ID NO: 8 - DNA, 18 bp, Sus scrofa
ggcccacgggcagcgcca

SEQ ID NO: 9 - skipped

SEQ ID NO: 10 - DNA, 16 bp, Sus scrofa
ccatgtgggaccggcc

SEQ ID NO: 11 - skipped

SEQ ID NO: 12 - DNA, 19 bp, Sus scrofa
ggcccgctggacgcgagtg

SEQ ID NO: 13 - DNA, 90 bp, Artificial Sequence
  Description of Artificial Sequence: Synthetic Repair template sequence
cgccccagccgcccggctcggctgaccctgcccggcgtggacgtgtcctggccccagctggtggtgccccggctggcgctgcccgtgggc

SEQ ID NO: 14 - DNA, 90 bp, Artificial Sequence
  Description of Artificial Sequence: Synthetic Repair template sequence
accctgcccggcgtggatgtgagccggccccagctggtggtgtaagcttccccggctggcgctgcccgtgggccactactgctttgtgtt

SEQ ID NO: 15 - DNA, 90 bp, Artificial Sequence
  Description of Artificial Sequence: Synthetic HDR repair template sequence
cggctcggctgaccctgcccggcgtggatgtgtctagacctcagctggtggtgccctggctggcgctgcccgtgggccactactgctttg

SEQ ID NO: 16 - DNA, 90 bp, Artificial Sequence
  Description of Artificial Sequence: Synthetic HDR repair template sequence
gcccggctcggctgaccctgcccggcgtggatgtctcgaggcctcaattggtggtgccccggctggcgctgcccgtgggccactactgct

SEQ ID NO: 17 - DNA, 90 bp, Artificial Sequence
  Description of Artificial Sequence: Synthetic HDR repair template sequence
gctctccatgtgggaccggccgcctcggagccgcttcacatgtgtccagagagccacctgctgtgccctcctcctctgcctcttcctggg

SEQ ID NO: 18 - DNA, 90 bp, Artificial Sequence
  Description of Artificial Sequence: Synthetic HDR repair template sequence
cctgcagggggcccaccctcctccacaacgtcttccctgcctaagcttcccgggggcggccctgctggggccccgaggacccctggcctc

SEQ ID NO: 19 - DNA, 90 bp, Artificial Sequence
  Description of Artificial Sequence: Synthetic HDR repair template sequence
ggcgtggatgtgagccggccccagctggtggtgccatggctcgccctccctgtcgggcactactgctttgtgttcgtggtgtcatttggc

SEQ ID NO: 20 - DNA, 18 bp, Artificial Sequence
  Description of Artificial Sequence: Synthetic PCR primer
cctcgcagccctctcaga

SEQ ID NO: 21 - DNA, 18 bp, Artificial Sequence
  Description of Artificial Sequence: Synthetic PCR primer
caccgaggccagacacac

SEQ ID NO: 22 - DNA, 20 bp, Artificial Sequence
  Description of Artificial Sequence: Synthetic PCR primer
tcctaccagaccgagtaccg

SEQ ID NO: 23 - DNA, 20 bp, Artificial Sequence
  Description of Artificial Sequence: Synthetic PCR primer
gctgagatcagagccaccag

SEQ ID NO: 24 - DNA, 19 bp, Artificial Sequence
  Description of Artificial Sequence: Synthetic PCR primer
tggtggagaaggaggtgct

SEQ ID NO: 25 - DNA, 19 bp, Artificial Sequence
  Description of Artificial Sequence: Synthetic PCR primer
gaaggatcaggctgggctg

SEQ ID NO: 26 - DNA, 43 bp, Artificial Sequence
  Description of Artificial Sequence: Synthetic HDR template sequence
tcgcagggggcatgtggattaccgtgttcttcgatggtaggtg

SEQ ID NO: 27 - DNA, 90 bp, Artificial Sequence
  Description of Artificial Sequence: Synthetic HDR template sequence
aactggacatcagctgatcgagctccttccagtcccagaggcggccaagtgggaattctgtggcctgtgttcacctcagaaccaaatcgg

SEQ ID NO: 28 - DNA, 89 bp, Sus scrofa
aactggacatcagctgatcgagctccttccagtcccagaggcggcaagtgggaattctgtggcctgtgttcacctcagaaccaaatcgg

SEQ ID NO: 29 - DNA, 19 bp, Artificial Sequence
  Description of Artificial Sequence: Synthetic Screening primer
ggtagacaatgctgttggc

SEQ ID NO: 30 - DNA, 20 bp, Artificial Sequence
  Description of Artificial Sequence: Synthetic Screening primer
tgctcaactagacagcaggc

SEQ ID NO: 31 - DNA, 40 bp, Sus scrofa
tggtgccccggctggcgctgcccgtgggccactactgctt

SEQ ID NO: 32 - DNA, 40 bp, Artificial Sequence
  Description of Artificial Sequence: Synthetic HDR template sequence
tggtgccatggctcgccctccctgtcgggcactactgctt

SEQ ID NO: 33 - DNA, 40 bp, Artificial Sequence
  Description of Artificial Sequence: Synthetic HDR template sequence
tggtgtaagcttccccggctggcgctgcccgtgggccact

SEQ ID NO: 34 - DNA, 35 bp, Sus scrofa
cggccccagctggtggtgccccggctggcgctgcc

SEQ ID NO: 35 - protein, 12 aa, Sus scrofa
RPQLVVPRLALP

SEQ ID NO: 36 - DNA, 42 bp, Artificial Sequence
  Description of Artificial Sequence: Synthetic HDR template sequence
cggccccagctggtggtgtaagcttccccggctggcgctgcc

SEQ ID NO: 37 - protein, 13 aa, Artificial Sequence
  Description of Artificial Sequence: Synthetic HDR template sequence
RPQLVVASPAGAA

SEQ ID NO: 38 - DNA, 42 bp, Artificial Sequence; identical to SEQ ID NO: 36
  Description of Artificial Sequence: Synthetic HDR template sequence

SEQ ID NO: 39 - protein, 13 aa, Artificial Sequence
  Description of Artificial Sequence: Synthetic peptide
  Induced mutation resulting from HDR template sequence
RPQLVVASPAGAA

SEQ ID NO: 40 - DNA, 42 bp, Sus scrofa
cggccccagctggtggtgccccggctggcgctgcccgtgggc

SEQ ID NO: 41 - protein, 14 aa, Sus scrofa
RPQLVVPRLALPVG

SEQ ID NO: 42 - DNA, 42 bp, Artificial Sequence
  Description of Artificial Sequence: Synthetic HDR template sequence
cggccccagctggtgctgccatccctccccctccctgtcgcg

SEQ ID NO: 43 - protein, 14 aa, Artificial Sequence
  Description of Artificial Sequence: Synthetic Mutant allele sequence
RPQLVVPWLALPVG

SEQ ID NO: 44 - DNA, 41 bp, Artificial Sequence
  Description of Artificial Sequence: Synthetic Mutant allele sequence
cggccccagctggtggtgccaggctcgccctccctgtcggg

SEQ ID NO: 45 - protein, 14 aa, Artificial Sequence
  Description of Artificial Sequence: Synthetic peptide
  Mutation induced by HDR template
RPQLVVPWLALPVG

SEQ ID NO: 46 - DNA, 59 bp, Sus scrofa
tctgcgtgtccgcattgaacctgaagaaggcagcctcgcagggggaaacatggatcaca

SEQ ID NO: 47 - protein, 19 aa, Sus scrofa
LRVRIEPEEGSLAGGTWIT

SEQ ID NO: 48 - DNA, 76 bp, Sus scrofa
tcttgtctgcgtgtccgcattgaacctgaagaaggcagcctcgcagggggaacatggatcacagtcttttttgatg

SEQ ID NO: 49 - protein, 25 aa, Sus scrofa
SCLRVRIEPEEGSLAGGTWITVFFD

SEQ ID NO: 50 - DNA, 75 bp, Homo sapiens
cgtcacctgagtttacatattgaacctgaagaaggtagccttgcagggggaacgtggatcacagtcatttttgat

SEQ ID NO: 51 - protein, 25 aa, Homo sapiens
RHLSLHIEPEEGSLAGGTWITVIFD

SEQ ID NO: 52 - protein, 15 aa, Homo sapiens
EEGSLAGGTWITVIF

SEQ ID NO: 53 - protein, 15 aa, Sus scrofa
EEGSLAGGTWITVFF

SEQ ID NO: 54 - protein, 17 aa, Artificial Sequence
  Description of Artificial Sequence: Synthetic Mutant protein sequence
EEGSLAGGMWITVFFDG

SEQ ID NO: 55 - DNA, 43 bp, Artificial Sequence; identical to SEQ ID NO: 26
  Description of Artificial Sequence: Synthetic HDR template sequence

SEQ ID NO: 56 - protein, 15 aa, Artificial Sequence
  Description of Artificial Sequence: Synthetic Mutant protein sequence
LAGGMWITVFFDGRC

SEQ ID NO: 57 - DNA, 43 bp, Artificial Sequence; identical to SEQ ID NO: 26
  Description of Artificial Sequence: Synthetic Mutant allele sequence

SEQ ID NO: 58 - protein, 15 aa, Artificial Sequence
  Description of Artificial Sequence: Synthetic Mutant protein sequence
LAGGMWITVFFDGRC

SEQ ID NO: 59 - DNA, 43 bp, Artificial Sequence; identical to SEQ ID NO: 26
  Description of Artificial Sequence: Synthetic Mutant allele sequence

SEQ ID NO: 60 - protein, 15 aa, Artificial Sequence
  Description of Artificial Sequence: Synthetic Mutant protein sequence
LAGGMWITVFFDGRC

SEQ ID NO: 61 - DNA, 43 bp, Artificial Sequence; identical to SEQ ID NO: 26
  Description of Artificial Sequence: Synthetic Mutant allele sequence

SEQ ID NO: 62 - protein, 15 aa, Artificial Sequence
  Description of Artificial Sequence: Synthetic Mutant protein sequence
LAGGMWITVFFDGRC

SEQ ID NO: 63 - protein, 54 aa, Homo sapiens
RNSVIVATSSSFDCIQDKVKPHSANLTSTDRAPSNPRGGRIGILWPVFTSEPNQ

SEQ ID NO: 64 - protein, 54 aa, Sus scrofa
RNSVIVATSSSFDCIQDRVKPRSANWTSADRAPSSPRGGRVGILWPVFTSEPNR

SEQ ID NO: 65 - DNA, 94 bp, Sus scrofa
caactggacatcagctgatcgagctccttccagtcccagagggggtcgagttggtatattgtggcctgtgttcacctcagaaccaaatcggtgg

SEQ ID NO: 66 - protein, 30 aa, Sus scrofa
NWTSADRAPSSPRGGRVGILWPVFTSEPNR

SEQ ID NO: 67 - DNA, 90 bp, Artificial Sequence; identical to SEQ ID NO: 27
  Description of Artificial Sequence: Synthetic HDR template sequence

SEQ ID NO: 68 - protein, 30 aa, Artificial Sequence
  Description of Artificial Sequence: Synthetic Mutant protein sequence
NWTSADRAPSSPRGGQVGILWPVFTSEPNR

SEQ ID NO: 69 - DNA, 76 bp, Artificial Sequence
  Description of Artificial Sequence: Synthetic Mutant allele sequence
ccttccagtcccagaggcggccaagtgggaattctgtggcctgtgttcacctcagaaccaaatcggtggccccagg

SEQ ID NO: 70 - protein, 25 aa, Artificial Sequence
  Description of Artificial Sequence: Synthetic Mutant protein sequence
PSSPRGGQVGILWPVFTSEPNRWPQ

SEQ ID NO: 71 - DNA, 65 bp, Artificial Sequence
  Description of Artificial Sequence: Synthetic Mutant allele sequence
agtcccagagggggtcgagttggtatattgtggcctgtgttcacctcagaaccaaatcggtggcc

SEQ ID NO: 72 - protein, 22 aa, Artificial Sequence
  Description of Artificial Sequence: Synthetic Mutant protein sequence
QSQRGSSWYIVACVHLRTKSVA

SEQ ID NO: 73 - DNA, 65 bp, Artificial Sequence
  Description of Artificial Sequence: Synthetic Mutant allele sequence
agtcccagaggcggccaagtgggaattctgtggcctgtgttcacctcagaaccaaatcggtggcc

SEQ ID NO: 74 - protein, 22 aa, Artificial Sequence
  Description of Artificial Sequence: Synthetic Mutant protein sequence
QSQRRPSGNSVACVHLRTKSVA

SEQ ID NO: 75 - DNA, 65 bp, Artificial Sequence; identical to SEQ ID NO: 73
  Description of Artificial Sequence: Synthetic Mutant allele sequence

SEQ ID NO: 76 - protein, 22 aa, Artificial Sequence
  Description of Artificial Sequence: Synthetic Mutant protein sequence
QSQRRPSGNSVACVHLRTKSVA

SEQ ID NO: 77 - protein, 20 aa, Homo sapiens
IEPEEGSLAGGTWITVIFDG

SEQ ID NO: 78 - protein, 20 aa, Sus scrofa
IEPEEGSLAGGTWITVFFDG

SEQ ID NO: 79 - protein, 26 aa, Homo sapiens
TSTDRAPSNPRGGRIGILWPVFTSEP

SEQ ID NO: 80 - protein, 26 aa, Sus scrofa
TSADRAPSSPRGGRVGILWPVFTSEP

SEQ ID NO: 81 - protein, 18 aa, Homo sapiens
VVLQGEEPIEIRSGVSIH

SEQ ID NO: 82 - protein, 18 aa, Sus scrofa
VVLQGEEPVEIRSGVAIH

SEQ ID NO: 83 - skipped

SEQ ID NO: 84 - DNA, 17 bp, Artificial Sequence
  Description of Artificial Sequence: Synthetic Nucleotide binding sequence
catggataacctcttat

SEQ ID NO: 85 - skipped

SEQ ID NO: 86 - DNA, 17 bp, Artificial Sequence
  Description of Artificial Sequence: Synthetic Nucleotide binding sequence
tcaacaggctcctctcc

SEQ ID NO: 87 - skipped

SEQ ID NO: 88 - DNA, 17 bp, Artificial Sequence
  Description of Artificial Sequence: Synthetic DNA binding sequence
aacctcttatatgttgt

SEQ ID NO: 89 - skipped

SEQ ID NO: 90 - DNA, 16 bp, Artificial Sequence
  Description of Artificial Sequence: Synthetic Nucleotide binding sequence
gagcgtatttcaacag

SEQ ID NO: 91 - skipped

SEQ ID NO: 92 - DNA, 15 bp, Artificial Sequence
  Description of Artificial Sequence: Synthetic Nucleotide binding sequence
cttatatgttatcct

SEQ ID NO: 93 - skipped

SEQ ID NO: 94 - DNA, 16 bp, Artificial Sequence
  Description of Artificial Sequence: Synthetic Nucleotide binding sequence
gagcgtatttcaacag

SEQ ID NO: 95 - skipped

SEQ ID NO: 96 - DNA, 18 bp, Artificial Sequence
  Description of Artificial Sequence: Synthetic Nucleotide binding sequence
ggacatcagctgatcgag

SEQ ID NO: 97 - skipped

SEQ ID NO: 98 - DNA, 17 bp, Artificial Sequence
  Description of Artificial Sequence: Synthetic Nucleotide binding sequence
ataccaactcgaccccc

SEQ ID NO: 99 - skipped

SEQ ID NO: 100 - DNA, 17 bp, Artificial Sequence
  Description of Artificial Sequence: Synthetic Nucleotide binding sequence
cagctgatcgagctcct

SEQ ID NO: 101 - skipped

SEQ ID NO: 102 - DNA, 15 bp, Artificial Sequence
  Description of Artificial Sequence: Synthetic Nucleotide binding sequence
ataccaactcgaccc

SEQ ID NO: 103 - DNA, 22 bp, Artificial Sequence
  Description of Artificial Sequence: Synthetic Nucleotide binding sequence
ggattaccgtgttcttcgatgg

SEQ ID NO: 104 - DNA, 23 bp, Artificial Sequence
  Description of Artificial Sequence: Synthetic Nucleotide binding sequence
gcagcctcgcagggggaacatgg

SEQ ID NO: 105 - DNA, 23 bp, Artificial Sequence
  Description of Artificial Sequence: Synthetic Nucleotide binding sequence
ggggtcgagttggtatattgtgg

SEQ ID NO: 106 - DNA, 90 bp, Artificial Sequence
  Description of Artificial Sequence: Synthetic HDR template sequence
gcgtgtccgcattgaacctgaagaaggcagcctcgcagggggcatgtggattaccgtgttcttcgatggtaggtgcggtcttcctacagc

SEQ ID NO: 107 - DNA, 90 bp, Artificial Sequence; identical to SEQ ID NO: 27
  Description of Artificial Sequence: Synthetic HDR template sequence

SEQ ID NO: 108 - DNA, 20 bp, Artificial Sequence
  Description of Artificial Sequence: Synthetic PCR primer
tcttggaccacggacctact

SEQ ID NO: 109 - DNA, 20 bp, Artificial Sequence
  Description of Artificial Sequence: Synthetic PCR primer
ttggggttcctggagtacca

SEQ ID NO: 110 - protein, 24 aa, Homo sapiens
LPGVDVSRPRLVLPRLALPVGHYC

SEQ ID NO: 111 - protein, 24 aa, Sus scrofa
LPGVDVSRPQLVVPRLALPVGHYC

SEQ ID NO: 112 - protein, 21 aa, Homo sapiens
WDRPPRSRFTRIQRATCCVLL

SEQ ID NO: 113 - protein, 21 aa, Sus scrofa
WDRPPRSRFTRVQRATCCALL

SEQ ID NO: 114 - protein, 26 aa, Homo sapiens
CRGPTLLQHVFPASPGATLVGPHGPL

SEQ ID NO: 115 - protein, 26 aa, Sus scrofa
CRGPTLLHNVFPASPGAALLGPRGPL

SEQ ID NO: 116 - protein, 13 aa, Artificial Sequence
  Description of Artificial Sequence: Synthetic peptide
CRGPTLLHNVFPA

SEQ ID NO: 117 - protein, 14 aa, Artificial Sequence
  Description of Artificial Sequence: Synthetic peptide
ASRGRPCWGPEDPW

SEQ ID NO: 118 - protein, 13 aa, Sus scrofa
SLAGGTWITVFFD

SEQ ID NO: 119 - protein, 31 aa, Sus scrofa
NWTSADRAPSSPRGGRVGILWPVFTSEPNRW